(19) United States
(12) Patent Application Publication
(10) Pub. No.: US 2008/0261831 A1
(43) Pub. Date: Oct. 23, 2008

(54) Ligands and libraries of ligands

(76) Inventors: Jim Wells, Burlingame, CA (US); Dan Erlanson, San Francisco, CA (US); Andrew C. Braisted, San Francisco, CA (US)

Correspondence Address: CHOATE, HALL & STEWART LLP, TWO INTERNATIONAL PLACE, BOSTON, MA, 02110, US

(21) Appl. No.: 11/607,257

(22) Filed: Dec. 1, 2006

Related U.S. Application Data
(63) Continuation of application No. 10/214,419, filed on Aug. 5, 2002, now abandoned.
Application No. 10/214,419 is a continuation-in-part of application No. 09/981,547, filed on Oct. 17, 2001, now abandoned.
Application No. 09/981,547 is a division of application No. 09/105,372, filed on Jun. 26, 1998, now Pat. No. 6,335,155.
Application No. 10/214,419 is a continuation-in-part of application No. 09/990,421, filed on Nov. 21, 2001, now Pat. No. 6,919,178.
Application No. 09/990,421 is a continuation-in-part of application No. 10/121,216, filed on Apr. 10, 2002, now Pat. No. 6,998,233.
(60) Provisional application No. 60/310,725, filed on Aug. 7, 2001.

(51) Int. Cl. C40B 40/04

(52) U.S. Cl. 506/15

(57) ABSTRACT

The invention relates to variants of Target Biological Molecules (TBMs), such as proteins, peptides and other amino acid sequences that are modified to include cysteine residues at predetermined positions within the TBM. The position of amino acid residues within the TBM that are modified to be cysteine residues is selected for its proximity to ligand binding sites within the TBM. Once an amino acid residue, or the DNA encoding the residue, is modified to cysteine, the TBM linked to potential binding ligands by forming a covalent bond through the cysteine thiol (—SH) reactive group of the variant.

### FIELD OF THE INVENTION

The present invention relates generally to variants of target biological molecules (TBMs), methods used to create the variants, disulfide ligand libraries and methods of screening and detecting binding of library members with the variants and TBM's. More specifically, the invention relates to methods for producing a variant target biological molecule (TBM), such as a protein, that provides a thiol-containing amino acid residue near a site of interest. The thiol-containing amino acid residue can then be used to covalently tether a biologically active molecule to determine if the biologically active molecule has affinity for the TBM at the site of interest.

### BACKGROUND OF THE INVENTION

The drug discovery process usually begins with massive screening of compound libraries (typically hundreds of thousands of members) to identify modest affinity leads (K_{d}˜1 to 10 μM). Although some targets are well suited for this screening process, most are problematic because moderate affinity leads are difficult to obtain. Identifying and subsequently optimizing weaker binding compounds would improve the success rate, but screening at high concentrations is generally impractical because of compound insolubility and assay artifacts. Moreover, the typical screening process does not target specific sites for drug design, only those sites for which a high-throughput assay is available. Finally, many traditional screening methods rely on inhibition assays that are often subject to artifacts caused by reactive chemical species or denaturants.

Erlanson et al., Proc. Nat. Acad. Sci. USA 97:9367-9372 (2000), have recently reported a new strategy, called “tethering”, to rapidly and reliably identify small (˜250 Da) soluble drug fragments that bind with low affinity to a specifically targeted site on a protein or other macromolecule, using an intermediary disulfide “tether.” According to this approach, a library of disulfide-containing molecules is allowed to react with a cysteine-containing target protein under partially reducing conditions that promote rapid thiol exchange. If a molecule has even weak affinity for the target protein, the disulfide bond (“tether”) linking the molecule to the target protein will be entropically stabilized. The disulfide-tethered fragments can then be identified by a variety of methods, including mass spectrometry (MS), and their affinity improved by traditional approaches upon removal of the disulfide tether. See also PCT Publication No. WO 00/00823, published on Jan. 6, 2000.

Although the tethering approach of Erlanson et al. represents a significant advance in the rapid identification of small low-affinity ligands, there is a need for more flexible methods of modifying proteins so that their ligand binding properties can be investigated.

### SUMMARY OF THE INVENTION

The methods described herein provide powerful techniques for generating drug leads, and allowing the identification fragments that bind weakly, or with moderate binding affinity, to a target at sites near one. The ligand compounds discovered by these methods are valuable tools in rational drug design, which can be further modified and optimized using medicinal chemistry approaches and structure-aided design.

Embodiments of the invention include a strategy for creating variant forms of virtually any target biological molecule (TBM). These variant forms are screened so that variant amino acid residues (cys) placed near a site of interest, such as a ligand binding domain, will form a covalent disulfide bond with potential ligand binding partners. A plurality of potential ligand binding partners are ultimately linked and tested with the variant TBM for their ability to bind or form non-covalent complexes. The advantage of first forming a reversible covalent bond between the potential ligand binding partners and the variant TBM is the ability to detect weak binding compounds whose binding affinity might not otherwise detectable in a non-covalent binding assay.

This approach uses a technique of modifying the TBM to contain a reactive group, such as a thiol-containing amino acid residue, in a region close to a site of interest. It should be realized that “thiol-containing amino acids” include cysteine residues, and variants (i.e. include non-naturally occurring amino acids) of cysteine.

In one embodiment, the TBM is modified so that it contains a thiol-containing amino acid residue within 10 angstroms of the site of interest. The thiol-containing amino acid residue is capable of forming a reversible covalent bond (e.g. tether) with reactive groups (S—S) present on candidate ligand binding partners. In another embodiment, the TBM is modified so that an amino acid reside within a 10 Angstrom radius of the site of interest is converted to cysteine residue. Preferrably, the modified amino acid residue is one that is accessible to solvent, as described below.

Another embodiment of the invention is a library of disulfide-containing potential ligand binding partners represented by Structural Formula I-XIV listed below. These ligand binding partners are covalently bound to the TBM to identify those partners having binding affinity.

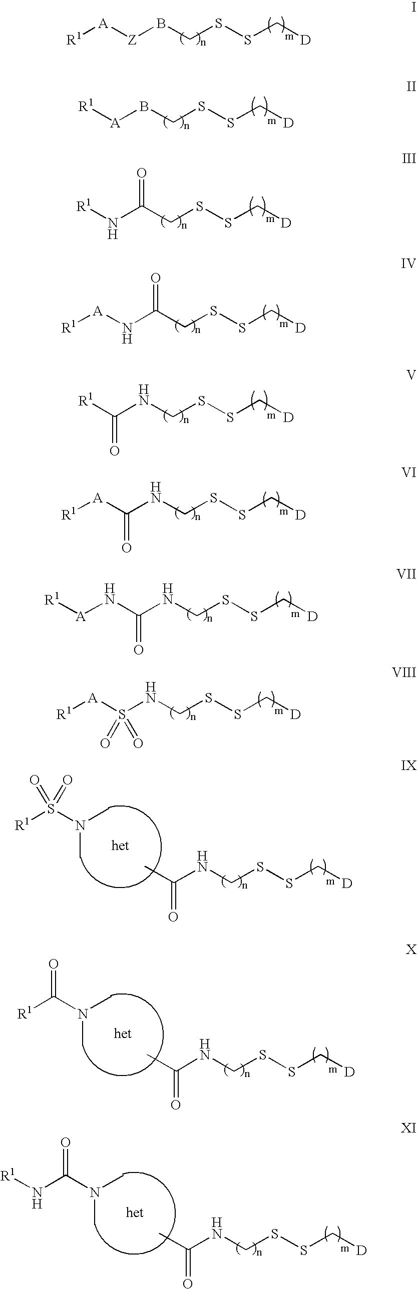

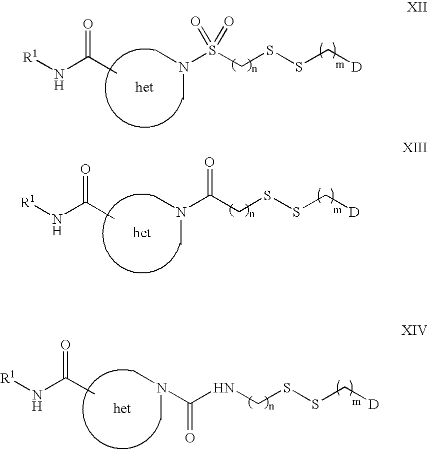

Where in compounds of Formula I-XIV:

A is absent or is independently B or is selected from -J-NR⁵—(CH₂)ₒ—, -J-NR⁵—CH(CH₃)—, -J-NR⁵—CH(CH₂C(═O)NHR²)—, -J-NR⁵—CH(CH₂CH₂C(═O)NHR²)—, -J-NR⁵—CH(CH₂NHR²)—, -J-NR⁵-het-(CH₂)_{q}—,

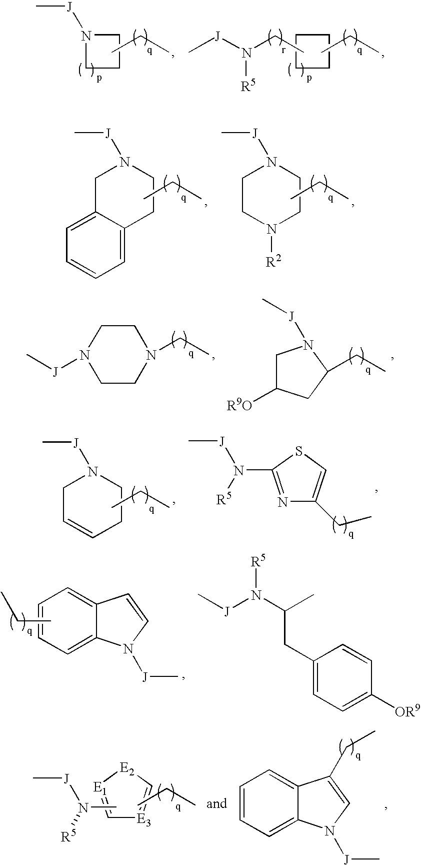

wherein any aryl or het may be substituted with 1-3 R⁴;

B is selected from —C(═O)—NH—, —C(═O)—NH—C(═O)—, —C(═O)—NH—SO₂—, —C(═O)—, C(═NH)—, —C(═NH)—SO₂—, —C(═O)—CH(NHR⁵)—, —C(═O)—CH(NHJ)-, —NR⁵—, —NR⁵—C(═O), —NR⁵—CH(C(═O)—R³)—, —NR⁵—CH(C(═O)—NR⁵)—, —SO₂—NH—, —SO₂—NH—C(═O)—, —NR⁵—SO₂—, —NR⁵—C(═O)—NH—, —NR⁵—C(═S)—NH—, —O—C(═O)—NH—, —NR⁵—C(═O)—O—, —O—C(═S)—NH—, —NR⁵—C(═S)—O—, —S—C(═O)—NH—, —NR⁵—C(═O)—S—, —S—C(═S)—NH—, —NR⁵—C(═S)—S—,

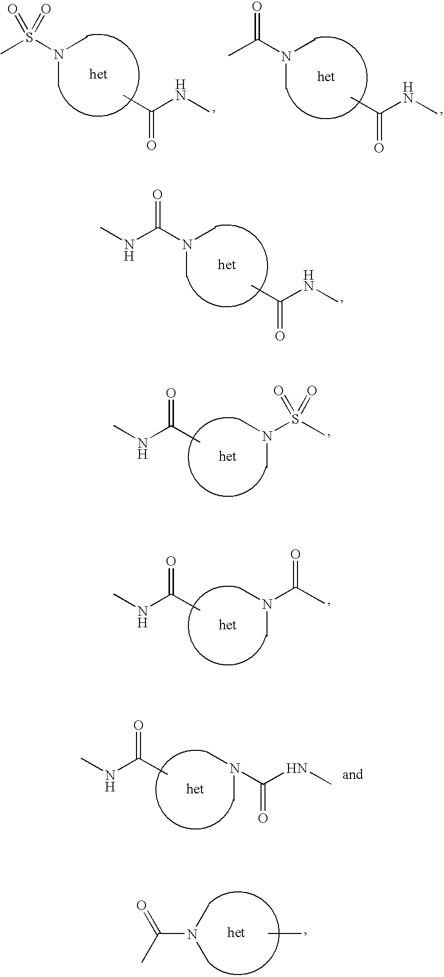

wherein

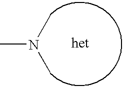

represents a het having at least one nitrogen in the position indicated, where any het may be substituted with 1-3 R⁴;

D is selected from —OH, —NH₂, —NHCOCH₃, —NHCONH₂, —NHCH₃, —N(CH₃)₂, —N(CH₃)₃, —COOH, —CONH₂, —SO₃H, —OPO₃H, —SO₂CH₃;

E₁, E₂, and E₃ are independently selected from NH, N, CH₂ and CH;

J is absent or is selected from hydrogen, OH, —C(═O)—, —NH—C(═O)—, —SO₂—, —NH—SO₂— and —NH—C(═S)—;

m and n are integers selected from 2 to 4;

is an integer selected from 1 and 2;

p is an integer selected from 1 to 3;

q and r are integers selected from 0 to 2;

R¹ or R² are selected from

hydrogen, C₁-C₆alkyl, C₀-C₄alkyl-C₃-C₁₁cycloalkyl, C₀-C₄alkyl-C₃-C₆cycloalkyl-C₆-C₁₂aryl, C₀-C₄alkyl-C₆-C₁₂aryl and C₀-C₄alkyl-het,

where

het is a heterocycle group composed of from 1 to 3 rings fused or linked sequentially, any ring independently being a saturated or unsaturated homocycle or heterocycle or a homo- or heteroaromatic, each ring containing 4, 5, 6 or 7 ring atoms and from 0-4 heteroatoms selected from N, O and S, provided at least one ring contains a heteroatom, where any homocycle or heterocycle ring N, S or C may optionally be oxidized and where any ring nitrogen may be substituted with R⁵ or R¹;

cycloalkyl may be a mono-, bi- or tricycle, where any nonadjacent cyclo-carbon may be oxidized to a ketone and where the cycloalkyl may optionally be fused to a C₆-C₁₂aryl, and where

any alkyl, cycloalkyl or heterocycle may be substituted with 1-3 R³ and any aryl or heteroaryl may be substituted with 1-3 R⁴;

R³ and R⁴ are independently selected from

C₁-C₆alkyl, C₁-C₆alkyloxy-C₀-C₆alkyl, C₁-C₆alkylthio-C₀-C₆alkyl,

C₁-C₆alkylcarbonyl-C₀-C₆alkyl, C₁-C₄alkylamino-C₀-C₆alkyl,

C₁-C₆alkylcarbonylamino-C₀-C₆alkyl, C₁-C₆alkyloxycarbonyl-C₀-C₆alkyl,

C₁-C₆alkyloxycarbonyl-C₁-C₆alkyloxy, C₁-C₄alkylcarbonylaminocarbonyl-C₀-C₆alkyl,

C₁-C₄alkylcarbonyloxy-C₀-C₆alkyl, C₁-C₄alkylcarbonylaminosulfonyl-C₀-C₆alkyl

C₁-C₄alkylaminocarbonyloxy-C₀-C₆alkyl, C₁-C₄alkylaminocarbonyl-C₀-C₆alkyl,

C₁-C₄alkylaminocarbonylamino-C₀-C₆alkyl, C₁-C₄alkylaminothiocarbonylamino-C₀-C₆alkyl, C₁-C₄alkylaminocarbonylaminosulfonyl-C₀-C₆alkyl, C₁-C₄alkylsulfonylaminocarbonylamino-C₀-C₆alkyl, C₁-C₄alkylsulfonylaminocarbonyl-C₀-C₆alkyl, di-(C₁-C₄alkyl)amino-C₁-C₄alkylaminocarbonyl, di-(C₁-C₄alkyl)amino-sulfonyl, C₁-C₄alkylaminosulfonyl-C₁-C₄alkyl, C₁-C₄alkyloxycarbonylamino-C₀-C₆alkyl, C₁-C₄alkyloxime, C₁-C₄alkylsulfonyl, C₃-C₁₁ cycloalkyl, C₃-C₁₁ cycloalkylamino, C₃-C₁₁ cycloalkylcarbonyl, C₃-C₁₁ cycloalkyloxy, C₃-C₁₁ cycloalkylthio, aminosulfonyl, aminocarbonyl, aminocarbonyl-C₁-C₄alkyl,

aminocarbonyl-C₁-C₄alkyloxy, amino-C₃-C₇ cycloalkyl, C₆-C₁₂ aryl-C₀-C₆alkyl,

C₆-C₁₂ aryloxy-C₀-C₆alkyl, C₆-C₁₂ arylthio-C₀-C₆alkyl,

C₆-C₁₂ arylcarbonyl-C₀-C₆alkyl, C₆-C₁₂ arylamino-C₀-C₆alkyl,

C₆-C₁₂ arylcarbonylamino-C₀-C₆alkyl, C₆-C₁₂ aryloxycarbonyl-C₀-C₆alkyl,

C₆-C₁₂ aryloxycarbonyl-C₁-C₆alkyloxy, C₆-C₁₂ arylcarbonylaminocarbonyl-C₀-C₆alkyl,

C₆-C₁₂ arylcarbonyl-C₁-C₆alkyloxycarbonyl-C₀-C₆alkyl, C₆-C₁₂ arylcarbonyl-C₁-C₆alkyloxy-C₀-C₆alkyl, C₆-C₁₂ arylcarbonylaminosulfonyl-C₀-C₆alkyl,

C₆-C₁₂ arylcarbonyloxy-C₀-C₆alkyl, C₆-C₁₂ arylaminocarbonyloxy-C₀-C₆alkyl,

C₆-C₁₂ arylaminocarbonyl-C₀-C₆alkyl, C₆-C₁₂ arylaminocarbonylamino-C₀-C₆alkyl,

C₆-C₁₂ arylaminothiocarbonylamino-C₀-C₆alkyl, C₆-C₁₂ arylaminocarbonylaminosulfonyl-C₀-C₆alkyl, C₆-C₁₂ arylsulfonylaminocarbonylamino-C₀-C₆alkyl, C₆-C₁₂ arylsulfonylaminocarbonyl-C₀-C₆alkyl, di-(C₆-C₁₂ aryl)amino-C₁-C₄alkylaminocarbonyl, C₆-C₁₂ arylaminosulfonyl-C₀-C₄alkyl,

C₆-C₁₂ aryloxycarbonylamino-C₀-C₆alkyl, C₆-C₁₂ aryloxime, C₆-C₁₂ aryl-C₁-C₄alkylaminocarbonyl-C₀-C₆alkyl, C₆-C₁₂ arylsulfonyl-C₀-C₆alkyl,

C₆-C₁₂ aryl-C₁-C₄alkylsulfonyl-C₀-C₆alkyl, C₆-C₁₂ aryl-C₁-C₄alkyloxy-C₀-C₆alkyl,

C₆-C₁₂ aryl-C₁-C₄alkyloxycarbonyl-C₀-C₆alkyl, C₆-C₁₂ aryl-C₁-C₄alkyloxycarbonylamino-C₀-C₆alkyl, carboxy-C₁-C₄alkyloxy, carboxy-C₁-C₄alkyl, carboxy-C₁-C₄alkylaminocarbonyl, carboxy-C₁-C₄alkylthio, carbonyl-C₁-C₄alkyl,

cyano-C₁-C₄alkyl, cyano-C₁-C₄alkylaminosulfonyl, het-C₀-C₆alkyl, het-oxy-C₀-C₆alkyl,

het-thio-C₀-C₆alkyl, het-carbonyl-C₀-C₆alkyl, het-amino-C₀-C₆alkyl, het-carbonylamino-C₀-C₆alkyl, het-oxycarbonyl-C₀-C₆alkyl, het-oxycarbonyl-C₁-C₆alkyloxy, het-carbonylaminocarbonyl-C₀-C₆alkyl, het-carbonylaminosulfonyl-C₀-C₆alkyl, het-carbonyloxy-C₀-C₆alkyl, het-aminocarbonyloxy-C₀-C₆alkyl,

het-aminocarbonyl-C₀-C₆alkyl, het-aminocarbonylamino-C₀-C₆alkyl,

het-aminothiocarbonylamino-C₀-C₆alkyl, het-aminocarbonylaminosulfonyl-C₀-C₆alkyl,

het-sulfonylaminocarbonylamino-C₀-C₆alkyl, het-sulfonylaminocarbonyl-C₀-C₆alkyl,

(het),(C₆-C₁₂ aryl)amino-C₁-C₄alkylaminocarbonyl, het-aminosulfonyl-C₀-C₄alkyl,

het-oxycarbonylamino-C₀-C₆alkyl, het-oxime, het-C₁-C₄alkylaminocarbonyl,

het-sulfonyl, het-C₁-C₄alkyloxycarbonyl, het-C₁-C₄alkyloxycarbonylamino,

hydroxy-C₁-C₆alkylcarbonyl, hydroxy-C₁-C₆alkyl, hydroxy-C₁-C₆alkyloxy,

hydroxy-C₆-C₁₀aryl, hydroxy-C₆-C₁₀aryloxyhydroxy-C₁-C₄alkyloxy,

morpholinyl, NR⁵R⁶, C(═NR⁵)—NR⁵R⁶ and pthalamidyl,

where any alkyl or cycloalkyl may be substituted with 1-3 R⁷ and any aryl or heteroaryl may be substituted with 1-3 R⁸;

R⁵ and R⁶ are independently selected from

hydrogen, het-C₀-C₄alkyl, het-C₀-C₄alkylcarbonyl, het-C₀-C₄alkyloxycarbonyl,

het-C₀-C₄alkylaminocarbonyl, het-C₀-C₄alkylsulfonyl, C₁-C₄alkyl, C₁-C₄alkylcarbonyl,

C₀-C₄alkyl-C₆-C₁₀arylcarbonyl, C₁-C₄alkylsulfonyl, C₁-C₄alkyloxy-C₀-C₄alkyl,

C₁-C₄alkyloxy-C₀-C₄alkylcarbonyl, C₁-C₄alkylamino-C₀-C₄alkylcarbonyl,

C₆-C₁₀aryl-C₀-C₄alkyl, C₆-C₁₀aryl-C₀-C₄alkylcarbonyl, C₆-C₁₀aryl-C₀-C₄alkyloxycarbonyl, C₆-C₁₀aryl-C₀-C₄alkylamino-C₀-C₄alkylcarbonyl, C₆-C₁₀aryl-C₀-C₄alkylsulfonyl, C₀-C₄alkylaminocarbonyl, C₆-C₁₀aryl-aminocarbonyl, C₃-C₁₀ cycloalkyl, C₃-C₁₀ cycloalkylcarbonyl, C₆-C₁₀aryl-C₃-C₁₀ cycloalkylcarbonyl,

C(═NH)—NH₂, acetyl, benzoyl, morpholino-C₁-C₄alkyl and C₁-C₄alkylcarbonyl;

where R⁵ and R¹ together with the nitrogen to which they are bonded may form a heterocycle containing up to three heteroatoms selected from N, S and O, where the heterocycle may be substituted with R³ and R⁴; and where

R⁵ and R⁶ together with the nitrogen to which they are bonded may form a morpholinyl or piperizinyl,

R⁷ is selected from halo (F, Cl, Br and I), carbonyl, hydroxy, nitro, cyano, methylsulfonyl, aminothiocyanate and benzoyl;

R⁸ is selected from amino, di-(C₁-C₄alkyl)amino-C₁-C₄alkyl, aminocarbonyl,

aminosulfonyl, methoxy, hydroxy, C₁-C₄alkyl, C₁-C₄alkylthio, C₁-C₄alkyloxycarbonyl,

C₁-C₄alkylcarbonylamino, carboxy, carboxyC₁-C₄alkyl, carboxyC₁-C₄alkylthio, acetamide, acetyl, halo, nitro, cyano, trifluoromethyl and formyl;

R⁹ is selected from hydrogen and —C(═O)NHR¹; and

Z is selected from C₁-C₁₁alkyl, C₀-C₄alkyl-C₃-C₁₁cycloalkyl-C₀-C₄alkyl, C₀-C₄alkyl C₃-C₆cycloalkyl-C₆-C₁₂aryl-C₀-C₄alkyl, C₀-C₄alkyl-C₆-C₁₂aryl-C₀-C₄alkyl,

and C₀-C₄alkyl-het-C₀-C₄alkyl.

Another embodiment of the invention is compounds of Formula I-XIV which are contacted with one or a plurality of Target Biological Molecules containing, or mutated to contain, a free thiol under conditions suitable for forming a disulfide complex of Formula XV and XVI;

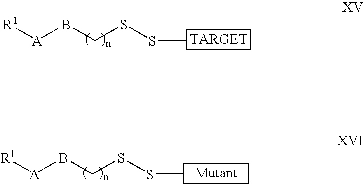

where the substituents R¹, A, B, and n are as defined above.

### BRIEF DESCRIPTION OF THE DRAWINGS

FIG. 1 is a schematic illustration of the basic tethering approach for site-directed ligand discovery.

FIG. 2 is a schematic illustration of an extended tethering approach.

FIG. 3 illustrates the chemical synthesis of a specific extender (2,6-dichloro-benzoic acid 3-(2-acetylsulfanyl-acetylamino)-4-carboxy-2-oxo-butyl ester), as described below in the Examples.

FIG. 4 shows the structural comparison between a known tetrapeptide inhibitor of caspase and a generic extender synthesized based on the inhibitor.

FIG. 5 shows mass spectra of two representative extended tethering experiments.

FIG. 6 is an illustration of a map of the pRSET/IL2 vector.

FIG. 7 shows exemplary ligands that bind to the Q78C variant of the human IL-4 molecule.

FIG. 8 shows exemplary ligands that bind to the E9C variant of the human IL-4 molecule.

FIG. 9 shows exemplary ligands that bind to the S16C variant of the human IL-4 molecule.

FIG. 10 is a schematic illustration of a basic “catch and release” approach used in the site directed ligand discovery process for targets that are heterogeneous due to glycosylation, high internal disulfide content or are otherwise heterogeneous or for proteins that do not fly well in a mass spectrometer. The process takes Targets that have been screened for hits with pools of disulfide library members that have been mass encoded, referred to as the tether reaction, and loads the Target-caught ligand complexes on a cation exchange column followed by rinsing, contacting with a reducing agent (e.g. TCEP) to release the caught ligand, separating the reducing agent from the released (thiol) ligand, optionally tagging the released ligand with a fluorophore such as rhodamine, and quantitating by fluorescence and/or mass spectroscopy.

FIG. 11 is a schematic illustration of the rhodamine exchange column used to quantitate and identify the ligand caught by a tether reaction. Released ligand is contacted with a rhodamine exchange column containing a disulfide bound rhodamine capable of exchanging with the released thiol to form a ligand (compound)-rhodamine complex.

### DETAILED DESCRIPTION OF CERTAIN PREFERRED EMBODIMENTS OF THE INVENTION

1. Overview

One embodiment of the invention relates to variants of Target Biological Molecules, such as proteins, peptides and other amino acid sequences as defined below. These variants are modified forms of a native Target Biological Molecule (TBM) and are useful for determining ligands that bind to native TBMs. As described in detail below, the variant TBMs preferably include thiol-containing amino acids, such as cysteine, which are capable of forming a reversible covalent bond through the cysteine thiol (—SH) reactive group of the variant. In addition, the variants may be derivatized with a reporter molecule for detecting the binding with other (non-covalent) library members.

The location of the thiol-containing amino acid residue within the TBM is chosen to be near a “site of interest”. Broadly, the “Site of Interest” on a particular target biological molecule is defined by the amino acid residues involved in binding of the target biological molecule to a molecule with which it forms a natural complex in vivo or in vitro.

When, for example, the target biological molecule is a protein that exerts its biological effect through binding to another protein, such as with hormones, cytokines or other proteins involved in signaling, it may form a natural complex in vivo with one or more other proteins. In this case the site of interest is defined as the critical contact residues involved in a particular protein:protein binding interface. Critical contact residues are defined as those amino acids on protein A that make direct contact with amino acids on protein B, and when mutated to alanine decrease the binding affinity by at least 10 fold and preferably at least 20 fold, as measured with a direct binding or competition assay (e.g. ELISA or RIA). See (A Hot Spot of Binding Energy in a Hormone-Receptor Interface by Clackston and Wells Science 267 383-386 (1995) and Cunningham and Wells J. Mol. Biol, 234 554-563 (1993)). Also included in the definition of a site of interest are amino acid residues from protein B that are within about 4 angstroms of the critical contact residues identified in protein A.

Scanning amino acid analysis can be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244: 1081-1085 (1989)). Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isoteric amino acid can be used.

However, it should be realized that those amino acid residues within protein A or protein B which, when mutated to alanine, result in a significant disruption of the secondary or tertiary structure of the protein are not preferred targets for modification by the methods disclosed herein. Examples of residues that may not be preferred modification targets include proline residues, particularly those participating in a cis amide bond.

When the target biological molecule is an enzyme, the site of interest can include amino acids that make contact with, or lie within, about 4 angstroms of a bound substrate, inhibitor, activator, cofactor or allosteric modulator of the enzyme. By way of illustration, when the enzyme is a protease, the site of interest would include the substrate binding channel from P4 to P4′, residues involved in catalytic function (e.g. the catalytic triad) and any cofactor (e.g. Zn) binding site. For protein kinases, the site of interest would include the substrate-binding channel (as above) in addition to the ATP binding site. For dehydrogenases, the site of interest would include the substrate binding region as well as the site occupied by NAD/NADH. In hydrolases such as PDE4, the site of interest would include all residues contacting the cAMP substrate, as well as residues involved in binding the catalytic divalent cations (Xu, R. X. et al. Science 288, 1822-1825 (2000)).

For an allosterically regulated enzyme, such as glycogen phosphorylase B, the site of interest includes all residues in the substrate binding region, residues in contact with the natural allosteric inhibitor glucose-6-phosphate, and residues in novel allosteric sites such as those identified in binding other inhibitors such as CP320626 (Oikonomakos N G, et al. Structure Fold Des 8, 575-584 (2000)).

In addition to the modified TBMs, embodiments of the invention include methods of modifying native TBMs to include the thiol-containing amino acid residues near a site of interest. For example, an embodiment of the invention includes selecting a TBM to modify, and then calculating the site of interest for that TBM. Once the site of interest is known, a process of determining which amino acid residue within, or near, the site of interest to modify is undertaken. As discussed below, one preferred modification results in substituting a cysteine residue for another amino acid residue located near the site of interest.

The choice of which residue within, or near, the site of interest to modify is determined based on the following selection criteria. First, a three dimensional description of the TBM is obtained from one of several well-known sources. For example, the tertiary structure of many TBMs has been determined through x-ray crystallography experiments. These x-ray structures are available from a wide variety of sources, such as the Protein Databank (PDB) which can be found on the Internet at http://www.rcsb.org. Tertiary structures can also be found in the Protein Structure Database (PSdb) which is located at the Pittsburgh Supercomputer Center at http://www.psc.com.

In addition, the tertiary structure of many proteins, and protein complexes, has been determined through computer-based modeling approaches. Thus, models of protein three-dimensional conformations are now widely available.

Once the three dimensional structure of the TBM is known, a measurement is made based on a structural model of the wild type, or a variant form, of the target biological molecule from any atom of an amino acid within the site of interest across the surface of the protein for a distance of approximately 10 angstroms. The variants produced by the methods described herein are the result of identifying a wild-type amino acid on the surface of the target biological molecule that falls within that approximate 10-angstrom radius from the site of interest. For the purposes of this measurement, any amino acid having at least one atom falling with in the about 10 angstrom radius from any atom of an amino acid within the site of interest is a potential residue to be modified to a thiol containing residue. The residues that fall within a 10 angstrom radius of the site of interest are referred to herein as the “10-angstrom variants”.

Preferred residues for modification are 10-angstrom variants that are solvent-accessible. Solvent accessibility may be calculated from structural models using standard numeric (Lee, B. & Richards, F. M. J. Mol. Biol. 55, 379-400 (1971); Shrake, A. & Rupley, J. A. J. Mol. Biol. 79, 351-371 (1973)) or analytical (Connolly, M. L. Science 221, 709-713 (1983); Richmond, T. J. J. Mol. Biol. 178, 63-89 (1984)) methods. For example, a potential cysteine variant is considered solvent-accessible if the combined surface area of the carbon-beta (CB), or sulfur-gamma (SG) is greater than 21 Å² when calculated by the method of Lee and Richards (Lee, B. & Richards, F. M. J. Mol. Biol. 55, 379-400 (1971)). This value represents approximately 33% of the theoretical surface area accessible to a cysteine side-chain as described by Creamer et al. (Creamer, T. P. et al. Biochemistry 34, 16245-16250 (1995)).

It is also preferred that the residue to be mutated to cysteine, or another thiol-containing amino acid residue, not participate in hydrogen-bonding with backbone atoms or, that at most, it interacts with the backbone through only one hydrogen bond. Wild-type residues where the side-chain participates in multiple (>1) hydrogen bonds with other side-chains are also less preferred. Variants for which all standard rotamers (chi1 angle of −60°, 60°, or 180°) can introduce unfavorable steric contacts with the N, CA, C, O, or CB atoms of any other residue are also less preferred. Unfavorable contacts are defined as interatomic distances that are less than 80% of the sum of the van der Waals radii of the participating atoms.

Wild-type residues that fall within highly flexible regions of the protein are less preferred. Within structures derived from x-ray data, highly flexible regions can be defined as segments where the backbone atoms possess weak electron density or high temperature factors (>4 standard deviations above the mean temperature factor for the structure). Within structures derived from NMR data, highly flexible regions can be defined as segments possessing <5 experimental restraints (derived from distance, dihedral coupling, and H-bonding data) per residue, or regions displaying a high variability (>2.0 Å2 RMS deviation) among the models in the ensemble. Additionally, residues found on convex “ridge” regions adjacent to concave surfaces are more preferred while those within concave regions are less preferred cysteine residues to be modified. Convexity and concavity can be calculated based on surface vectors (Duncan, B. S. & Olson, A. J. Biopolymers 33, 219-229 (1993)) or by determining the accessibility of water probes placed along the molecular surface (Nicholls, A. et al. Proteins 11, 281-296 (1991); Brady, G. P., Jr. & Stouten, P. F. J. Comput. Aided Mol. Des. 14, 383-401 (2000)). Residues possessing a backbone conformation that is nominally forbidden for L-amino acids (Ramachandran, G. N. et al. J. Mol. Biol. 7, 95-99 (1963); Ramachandran, G. N. & Sasisekharahn, V. Adv. Prot. Chem. 23, 283-437 (1968)) are less preferred targets for modification to a cysteine. Forbidden conformations commonly feature a positive value of the phi angle.

Other preferred variants are 10-angstrom variants which, when mutated to cysteine and linked via a disulfide bond to an alkyl tether, would possess a conformation that directs the atoms of that tether towards the site of interest. Two general procedures can be used to identify these preferred variants. In the first procedure, a search is made of unique structures (Hobohm, U. et al. Protein Science 1, 409-417 (1992)) in the Protein Databank (Berman, H. M. et al. Nucleic Acids Research 28, 235-242 (2000)) to identify structural fragments containing a disulfide-bonded cysteine at position j in which the backbone atoms of residues j−1, j, and j+1 of the fragment can be superimposed on the backbone atoms of residues i−1, i, and i+1 of the target molecule with an RMSD of less than 0.75 Å². If fragments are identified that place the CB atom of the residue disulfide-bonded to the cysteine at position j closer to any atom of the site of interest than the CB atom of residue i (when mutated to cysteine), position i is considered preferred. In an alternative procedure, the residue at position i is computationally “mutated” to a cysteine and capped with an S-Methyl group via a disulfide bond.

Potential conformations of the capped cysteine residue are enumerated based on standard rotamers (Ponder, J. W. and Richards, F. M. J. Mol. Biol. 193, 775-791 (1987)) or by exhaustive conformational searching. Each potential conformation is then energy-minimized in the presence of the entire protein using standard forcefield methods. (Weiner, S. J. et al. J. Comput. Chem. 7, 230-252 (1986); Nemethy, G. et al. J. Phys. Chem. 96, 6472-6484 (1992); Brooks, B. R. et al. J. Comput. Chem. 4, 187-217 (1983)).

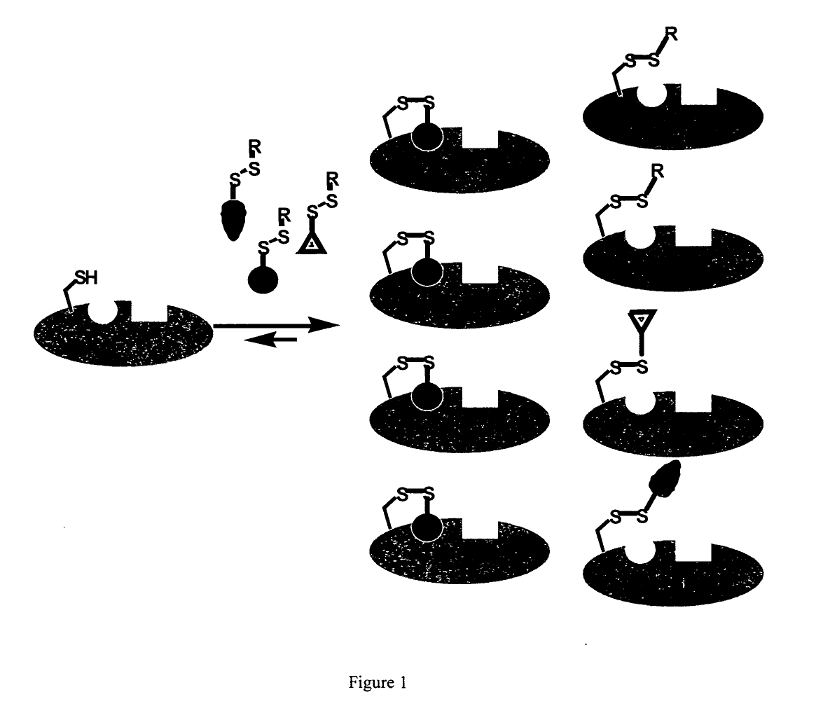

All minimized conformations within 5.0 kcal/mol of the lowest-energy conformation are considered accessible. If any accessible conformation places the methyl carbon of the S-Methyl group closer to any atom of the site of interest than the CB atom of residue i (when mutated to cysteine), position i is considered preferred. One exemplary approach to tethering is illustrated in FIG. 1.

As indicated in FIG. 1, a target molecule, containing or modified to contain a free thiol group (such as a cysteine-containing protein) is equilibrated with a disulfide-containing library in the presence of a reducing agent, such as 2-mercaptoethanol. Most of the library members will have little or no intrinsic affinity for the target molecule, and thus by mass action the equilibrium will lie toward the unmodified target molecule. However, if a library member does show intrinsic affinity for the target molecule, the equilibrium will shift toward the modified target molecule, having attached to it the library member with a disulfide tether.

Other embodiments of the invention include the following protein variants, and DNA encoding the protein variants, that have been modified to include cysteine residues in place of amino acid residues within 10 angstroms of a site of interest. Examples of such proteins include variants of human Interleukin-2 (IL-2) listed below. Within the following nomenclature identifying each variant, the first character is the single amino acid code for the native amino acid, the numeral is the position of the amino acid in the native protein, and the last character is the substituted amino acid in the variant form of the protein. For example, in the following variants, the letter “C” indicates that the native amino acid residue was replaced with a cysteine residue. The DNA sequence of each variant can be determined by analyzing the gene that encodes each variant, and then calculating the codon within the gene the encodes the amino acid residue to be altered to, for example, cysteine. Accordingly, if the variant is listed as N30C, one would look to the DNA codon that encodes the N at position 30, and then perform a mutagenesis procedure on the DNA, as described in the Examples below, to alter the codon for N to a codon for C.

The following variants of IL-2 that have one or more of the indicated modifications are included within the scope of the invention: N30C, Y31C, N33C, K32C, K35C, R38C, F42C, K43C, Y45C, E68C, L72C, N77C, Y31C, K43C, L72C, and K43C. In addition, DNA that has been mutagenized to encode one or more of these IL-2 variants is within the scope of the invention.

Another embodiment of the invention is modified human Caspase-3, which includes one or more of the following variants of the small subunit of the molecule: F256C, S209C, S251C, W214C, and Y204C. Embodiments of the invention also include proteins that have one or more of the following variants of the large subunit of human Caspase-3: H121C, L168C, and S65C. In addition, DNA that has been mutagenized to encode one or more of these Caspase-3 variants is within the scope of the invention.

Yet another embodiment of the invention is modified human type I Interleukin-1 receptor protein having one or more of the following variants: E11C, I13C, V16C, K112C, Q113C, V124C, and E129C. In addition, DNA that has been mutagenized to encode one or more of these type 1 Interleukin 1 variants is within the scope of the invention.

One other embodiment of the invention is variant forms of the human β-secretase protein that have one or more of the following variations: T72C, Q73C, F108C, I110C, R128C, Y198C, N233C, R235C, and T329C. In addition, DNA that has been mutagenized to encode one or more of these human β-secretase variants is within the scope of the invention.

Still another embodiment of the invention is HIV integrase that has been modified to include one or more of the following modifications: Q62C, H67C, E92C, D116C, N120C, H114C, E152C, K156C and C165. In addition, DNA that has been mutagenized to encode one or more of these HIV integrase variants is within the scope of the invention.

Another embodiment of the invention is modified Tumor Necrosis Factor-α (TNF-α) that includes one or more of the following modifications: R32C, A33C, T77C, V91C, Q125C, and S147C. In addition, DNA that has been mutagenized to encode one or more of these TNF-α variants is within the scope of the invention.

Still another embodiment of the invention is modified human Interleukin-4 alpha receptor that includes one or more of the following modifications: M14C, L39C, L43C, H47C, D66C, D67C, V69C, S70C, D72C, K97C, R99C and D125C. In addition, DNA that has been mutagenized to encode one or more of these human IL-4 alpha receptor variants is within the scope of the invention.

One other embodiment of the invention is variant forms of the human IL-4 protein that have one or more of the following variations: T6C, Q8C, E9C, K12C, N15C, S16C, K37C, N38C, K42C, Q54C, Q78C, R81C, R85C, R88C, N89C, N97C, K102C, K117C and R121C. Preferred variations include the following mutations: E9C, S16C and Q78C. In addition, DNA that has been mutagenized to encode one or more of these human IL-4 variants is within the scope of the invention.

Also preferred embodiments of the invention include cysteine mutants or variants where any naturally occurring free cysteine is substituted with another amino acid, especially alanine.

### DEFINITIONS

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “target” is used in the broadest sense and refers to a chemical or biological entity for which a ligand has intrinsic binding affinity. The target can be a molecule, a portion of a molecule, or an aggregate of molecules. The target is capable of reversible attachment to a ligand via a tether. Specific examples of target molecules include polypeptides (e.g., enzymes, receptors, transcription factors, ligands for receptors, growth factors, immunoglobulins, nuclear proteins, signal transduction components, allosteric enzyme regulators, and the like), polynucleotides, peptides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. The definition specifically includes Target Biological Molecules (TBMs) as defined below.

The term “polynucleotide”, when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

A “ligand” as defined herein is an entity that has an intrinsic binding affinity for the target. The ligand can be a molecule, or a portion of a molecule that binds the target. The ligands are typically small organic molecules which have an intrinsic binding affinity for the target molecule, but may also be other sequence-specific binding molecules, such as peptides (D-, L- or a mixture of D- and L-), peptidomimetics, complex carbohydrates or other oligomers of individual units or monomers which bind specifically to the target. The term “monophore” is used herein interchangeably with the term “ligand” and refers to a monomeric unit of a ligand. The term “diaphore” denotes two monophores covalently linked to form a unit that has a higher affinity for the target because of the two constituent monophore units or ligands binding to two separate but nearby sites on the target. The binding affinity of a diaphore that is higher than the product of the affinities of the individual components is referred to as “avidity.” The term diaphore is used irrespective of whether the unit is covalently bound to the target or existing separately after its release from the target. The term also includes various derivatives and modifications that are introduced in order to enhance binding to the target.

(A) The “Site of Interest” on a particular target biological molecule is defined as the amino acid residues involved in binding of the Target Biological Molecule to a molecule with which it forms a natural complex in vivo. A “site of interest” on a TBM is a group of amino acid residues to which a specific in vivo binding molecule (e.g. substrate, inhibitor, effector or cofactor) binds, which may include a specific sequence of monomeric subunits, e.g. amino acid residues, or nucleotides, and may have a three-dimensional structure. Typically, the molecular interactions between the in vivo binding molecule and the site of interest on the target are non-covalent, and include hydrogen bonds, van der Waals interactions and electrostatic interactions.

“Small molecules” are usually less than 1 kDa molecular weight, and include but are not limited to non-peptidic synthetic organic compounds. Preferred small molecules have molecular weights of less that 300 Da and more preferably less than 650 Da.

The term “tether” as used herein refers to a structure which includes a moiety capable of forming a reversible covalent bond with a target (including Target Biological Molecules as hereinafter defined), near a site of interest. Optionally, the tether may contain a spacer element, such as an alkane group.

The phrase “reversible covalent bond” as used herein refers to a covalent bond linking a ligand to a TBM which can be broken, preferably under conditions that do not denature the target molecule. Examples include, without limitations, disulfides, Schiff bases, thioesters, and the like.

The term “reactive group” with reference to a ligand is used to describe a chemical group or moiety providing a site at which covalent bond with the ligand candidates (e.g. members of a library or small organic compounds) may be formed. Thus, the reactive group is chosen such that it is capable of forming a covalent bond with members of the library against which it is screened.

A “Target Biological Molecule” or “TBM” as used herein refers to a single biological molecule or a plurality of biological molecules capable of forming a biologically relevant complex with one another. Preferably, the TBM is a target for which a small molecule agonist or antagonist would have therapeutic importance. In a preferred embodiment, the TBM is a polypeptide, especially a protein, that possesses, or is capable of being modified to possess, a reactive group for binding to members of a library of small organic molecules.

The term “antagonist” is used in the broadest sense and includes any ligand that partially or fully blocks, inhibits or neutralizes a biological activity exhibited by a target, such as a TBM. In a similar manner, the term “agonist” is used in the broadest sense and includes any ligand that mimics a biological activity exhibited by a target, such as a TBM, for example, by specifically changing the function or expression of such TBM, or the efficiency of signaling through such TBM, thereby altering (increasing or inhibiting) an already existing biological activity or triggering a new biological activity.

The phrase “modified to contain” and “modified to possess” are used interchangeably, and refer to making a mutant, variant, or derivative of the target, or the reactive nucleophile or electrophile, including, but not limited to chemical modifications. For example, in a protein, one can substitute an amino acid residue having a side chain containing a nucleophile or electrophile for a wild-type residue. Another example is the conversion of a thiol group of a cysteine residue to an amine group.

The term “reactive nucleophile” as used herein refers to a nucleophile that is capable of forming a covalent bond with a compatible functional group on another molecule under conditions that do not denature or otherwise damage the target, e.g. TBM. The most relevant nucleophiles are thiols, alcohols, activated carbonyls, epoxides, aziridines, aromatic sulfonates, hemiacetals, and amines. Similarly, the term “reactive electrophile” as used herein refers to an electrophile that is capable of forming a covalent group with a compatible functional group on another molecule, preferably under conditions that do not denature or otherwise damage the target, e.g. TBM. the most relevant electrophiles are imines, carbonyls, epoxides, aziridines, sulfonates, and hemiacetals.

The phase “small molecule extender” as used herein refers to a small organic molecule having a molecular weight of from about 75 to about 750 daltons and having a first functional group reactive with the nucleophile on the TBM and a second functional group that is a free or protected thiol or a group that is a precursor of a free of protected thiol, at least a portion of the small molecule extender being capable of forming a non-covalent bond with a first site of interest on the TBM.

A “first site of interest” on a TBM refers to a site that can be contacted by at least a portion of the small molecule extender when it is covalently bound to the reactive nucleophile and which is capable of forming a non-covalent interaction with the small molecule extender.

The phrases “group reactive with the nucleophile” and “nucleophile reactive group”, “group reactive with the electrophile” and “electrophile reactive group” as used herein refer to a functional group on the small molecule extender that can form a covalent bond with the nucleophile/electrophile on the TBM, preferably under conditions that do not denature or otherwise damage the TBM.

The term “protected thiol” as used herein refers to a thiol that has been reacted with a group or molecule to form a covalent bond that renders it less reactive and which may be de-protected to regenerate a free thiol.

The phrase “adjusting the conditions” as used herein refers to subjecting a target, such as a TBM to any individual, combination or series of reaction conditions or reagents necessary to cause a covalent bond to form between the ligand and the target, such as a nucleophile and the group reactive with the nucleophile on the small molecule extender, or to break a covalent bond already formed.

The term “covalent complex” as used herein refers to the combination of the small molecule, or small molecule extender, and the TBM.

The phrase “exchangeable disulfide linking group” as used herein refers to the library of molecules screened with the covalent complex displaying the thiol-containing small molecule extender, where each member of the library contains a disulfide group that can react with the thiol or protected thiol displayed on the covalent complex to form a new disulfide bond when the reaction conditions are adjusted to favor such thiol exchange.

The phase “highest affinity for the second site of interest” as used herein refers to the molecule having the greater thermodynamic stability toward the second site of interest on the TBM that is preferentially selected from the library of disulfide-containing library members.

“Functional Variants” of a molecule herein are variants having an activity in the common with the referenced molecule.

“Active” or “activity” means a qualitative biological and/or immunological property, wherein “immunological property” refers to the ability to induce the production of an antibody against an antigenic epitope possessed by a referenced molecule.

The term “alkyl” means a linear, branched or unbranched, alkane, alkene or alkyne hydrocarbon radical, having the number of carbon atoms specified, or if no number is specified, having up to 12 carbon atoms. Any carbon-carbon double bonds being independently cis, trans, or a non-geometric isomer. Examples of alkyl radicals include; methyl, ethyl, n-propyl, isopropyl (iPr), n-butyl, iso-butyl, sec-butyl, tert-butyl (tBu), n-pentyl, 2-methylbutyl, 2,2-dimethylpropyl, n-hexyl, 2-methylpentyl, 2,2-dimethylbutyl, n-heptyl, 2-methylhexyl, and the like. The terms “lower alkyl” and “C₁-C₆alkyl” are synonymous and used interchangeably.

The term “Cₙ-Cₘ alkyl” where n is 0 or an integer and m is an integer is a shorthand designation of the range of carbon atoms contained in the alkyl group. For the case where n is 0 the above term simply means a chemical bond when the C₀ alkyl group is between two other functional groups or is hydrogen when the C₀ alkyl is at the terminus.

Substituted alkyl groups may be substituted once, twice or three times with the same or with different substituents. Common examples of substituted alkyl groups include but are not limited to; cyanomethyl, nitromethyl, hydroxymethyl, trityloxymethyl, propionyloxymethyl, aminomethyl, carboxymethyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like.

The term “cycloalkyl” means a mono-, bi-, or tricyclic saturated, unsaturated or partially unsaturated aliphatic hydrocarbon radical having the number or range of carbon atoms specified, typically 3 to 14 carbon atoms and preferably 3 to 7 carbon atoms. An exemplary cycloalkyl is cyclohexyl. Optionally, any nonadjacent carbons bonded to only two other carbons through single bonds may be oxidized to a ketone.

The terms “C₁-C₆ alkyloxy” or “C₁-C₆alkoxy” are used interchangeably herein and denote groups such as methoxy, ethoxy, n-propoxy, isopropoxy, n-butoxy, t-butoxy, cyclohexyloxy and like groups.

The terms “C₁-C₁₂ acyloxy” or “C₁-C₁₂ alkanoyloxy” or “C₁-C₁₂ alkylcarbonyloxy” are used interchangeably and denote herein groups such as formyloxy, acetoxy, propionyloxy, butyryloxy, pentanoyloxy, hexanoyloxy, heptanoyloxy, and the like.

The terms “C₁-C₁₂ alkylcarbonyl”, “C₁-C₁₂ alkanoyl” and “C₁-C₁₂ acyl” are used interchangeably herein and encompass groups such as formyl, acetyl, propionyl, butyryl, pentanoyl, hexanoyl, heptanoyl, and the like.

The term “C₁-C₁₂ alkylthio” refers to alkyl groups, as defined above, attached or bonded to a sulfur which is in turn the point of attachment for the alkylthio to the group or substituent designated.

The term “aryl” when used alone means a homocyclic hydrocarbon aromatic radical, whether or not fused, having the number of carbon atoms designated. Preferred aryl groups include; phenyl, napthyl, biphenyl, phenanthrenyl, naphthacenyl, and the like (see e.g. Lang's Handbook of Chemistry (Dean, J. A., ed.) 13th ed. Table 7-2, 1985).

Examples of the term “substituted phenyl” includes but is not limited to a mono- or di(halo)phenyl group such as 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(iso-propyl)phenyl, 4-ethylphenyl, 3-(n-propyl)phenyl and the like; a mono or di(alkoxy)phenyl group, for example, 2,6-dimethoxyphenyl, 4-methoxyphenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))-phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like. Preferred substituted phenyl groups include the 2- and 3-trifluoromethylphenyl, the 4-hydroxyphenyl, the 2-aminomethylphenyl and the 3-(N-methylsulfonylamino))phenyl groups.

The term “arylalkyl” means one, two, or three aryl groups having the number of carbon atoms designated, appended to an alkyl radical having the number of carbon atoms designated including but not limited to; benzyl, napthylmethyl, phenethyl, benzhydryl(diphenylmethyl), trityl, and the like. A preferred arylalkyl group is the benzyl group.

The term “substituted C₆-C₁₂aryl-C₁-C₆ alkyl” denotes a C₁-C₆ alkyl group substituted at any carbon with a C₆-C₁₂ aryl group bonded to the alkyl group through any aryl ring position and substituted on the C₁-C₁₂ alkyl portion with one, two or three groups chosen from halogen (F, Cl, Br, I), hydroxy, protected hydroxy, amino, protected amino, C₁-C₆ acyloxy, nitro, carboxy, protected carboxy, carbamoyl, carbamoyloxy, cyano, C₁-C₆ alkylthio, N-(methylsulfonylamino) C₁-C₆ alkoxy, or other groups specified. Optionally, the aryl group may be substituted with one, two, or three groups chosen from halogen (especially F or Cl), cyano, hydroxy, protected hydroxy, nitro, C₁-C₆ alkyl, C₁-C₄ alkoxy, carboxy, protected carboxy, carboxymethyl, protected carboxymethyl, hydroxymethyl, protected hydroxymethyl, aminomethyl, protected aminomethyl, or an N-(methylsulfonylamino) group.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, t-butyl, t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta.-(trimethylsilyl)ethyl, beta.-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions and can be removed at the appropriate point without disrupting the remainder of the molecule. Preferred carboxylic acid protecting groups are the allyl and p-nitrobenzyl, groups. Similar carboxy-protecting groups used in the cephalosporin, penicillin and peptide arts can also be used to protect carboxy group substituents of the benzodiazepine. Further examples of these groups are found in E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted with one of the above carboxy-protecting groups.

As used herein the term “amide-protecting group” refers to any group typically used in the peptide art for protecting the peptide nitrogens from undesirable side reactions. Such groups include; BOC (t-butyloxycarbonyl), FMOC (fluorenylmethyloxycarbonyl), p-methoxyphenyl, 3,4-dimethoxybenzyl, benzyl, o-nitrobenzyl, di-(p-methoxyphenyl)methyl, triphenylmethyl, (p-methoxyphenyl)diphenylmethyl, diphenyl-4-pyridylmethyl, m-2-(picolyl)-N′-oxide, 5-dibenzosuberyl, trimethylsilyl, t-butyl dimethylsilyl, and the like. Further descriptions of these protecting groups can be found in “Protective Groups in Organic Synthesis”, by Theodora W. Greene, 1981, John Wiley and Sons, New York.

Unless otherwise specified, the terms “Het”, “het”, “heterocycle”, heterocyclic group”, “heterocyclic” or “heterocyclyl” are used interchangeably herein and refer to any mono-, bi-, or tricyclic saturated, unsaturated, or aromatic ring having the number of ring atoms designated or, if no number is indicated, from 1-3 rings, where at least one ring is a 4- 5-, 6- or 7-membered hydrocarbon ring containing from one to four heteroatoms selected from nitrogen, oxygen, and sulfur (Lang's Handbook: of Chemistry, supra). The other rings, if any, may be saturated, unsaturated or aromatic homocycle or heterocycle rings. Optionally, any carbon in a nonaromatic ring bonded to two other carbons through single bonds may be oxidized to a ketone and any N or S may also be oxidized. The term “heteroaryl” means a heterocycle having aromatic character. Preferably, the heterocycle is a 5- or 6-member saturated, unsaturated, or aromatic hydrocarbon ring containing 1, 2, or 3 heteroatoms selected from O, N, and S. Typically, the 5-membered ring has 0 to 2 double bonds and the 6- or 7-membered ring has 0 to 3 double bonds and the nitrogen or sulfur heteroatoms may optionally be oxidized, and any nitrogen heteroatom may optionally be quarternized. Included in the definition are any bicyclic groups where any of the above heterocyclic rings are fused to a benzene ring.

The following ring systems are examples of the heterocyclic (whether substituted or unsubstituted) radicals denoted by the term “heterocyclic”: thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo>1,5-bipyridazinyl and purinyl, as well as benzo-fused derivatives, for example benzoxazolyl, benzthiazolyl, benzimidazolyl and indolyl.

Heterocyclic 5-membered ring systems containing a sulfur or oxygen atom and one to three nitrogen atoms are also suitable for use in the instant invention. Examples of such groups include thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, preferably oxazol-2-yl, and oxadiazolyl, such as 1,3,4-oxadiazol-5-yl, and 1,2,4-oxadiazol-5-yl. A group of further examples of 5-membered ring systems with 2 to 4 nitrogen atoms include imidazolyl, preferably imidazol-2-yl; triazolyl, preferably 1,3,4-triazol-5-yl; 1,2,3-triazol-5-yl, 1,2,4-triazol-5-yl, and tetrazolyl, preferably 1H-tetrazol-5-yl. Another group of examples of benzo-fused rings are benzoxazol-2-yl, benzthiazol-2-yl and benzimidazol-2-yl.

Further suitable specific examples of the above heterocylic ring systems are 6-membered ring systems containing one to three nitrogen atoms. Such examples include pyridyl, such as pyrid-2-yl, pyrid-3-yl, and pyrid-4-yl; pyrimidyl, preferably pyrimid-2-yl and pyrimid-4-yl; triazinyl, preferably 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl radicals, are a preferred group. Optionally preferred 6-membered ring heterocycles are; piperazinyl, piperazin-2-yl, piperidyl, piperid-2-yl, piperid-3-yl, piperid-4-yl, morpholino, morpholin-2-yl, and morpholin-3-yl.

An optionally preferred group of “heterocyclics” include; 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo>1,5-b!pyridazin-6-yl and 8-aminotetrazolo>1,5-b!-pyridazin-6-yl.

An alternative group of “heterocyclics” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo>1,5-b!pyridazin-6-yl, and 8-aminotetrazolo>1,5-b!pyridazin-6-yl.

The terms “heteroaryl group” or “heteroaryl” are used interchangeably herein and refer to any mono-, bi-, or tricyclic aromatic rings having the number of ring atoms designated where at least one ring is a 5-, 6- or 7-membered hydrocarbon ring containing from one to four heteroatoms selected from nitrogen, oxygen, and sulfur, preferably at least one heteroatom is nitrogen. The aryl portion of the term “heteroaryl” refers to aromaticity, a term known to those skilled in the art and defined in greater detail in Advanced Organic Chemistry J. March, 3.sup.rd ed., pages 37-69, John Wiley & Sons, New York (1985).

The term “prodrug” as used herein means a pharmacologically inactive derivative of a parent drug molecule that requires biotransformation, either spontaneous or enzymatic, within the organism to release the active drug.

Targets

Targets, such as target biological molecules (TBMs), include, without limitation, molecules, portions of molecules and aggregates of molecules to which a ligand candidate may bind, such as polypeptides or proteins (e.g., enzymes, receptors, transcription factors, ligands for receptors, growth factors, immunoglobulins, nuclear proteins, signal transduction components, allosteric enzyme regulators, and the like), polynucleotides, peptides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. The target can be obtained in a variety of ways, including isolation and purification from natural source, chemical synthesis, recombinant production and any combination of these and similar methods.

In a particularly preferred embodiment, the target is a TBM, and even more preferably is a polypeptide, especially a protein. Polypeptides that find use herein as targets for binding ligands, preferably small organic molecule ligands, include virtually any polypeptide (including short polypeptides also referred to as peptides) or protein that comprises two or more binding sites of interest, and which possesses or is capable of being modified to possess a reactive group for binding to a small organic molecule or other ligand (e.g. peptide).

Polypeptides of interest may be obtained commercially, recombinantly, by chemical synthesis, by purification from natural source, or otherwise and, for the most parts are proteins, particularly proteins associated with a specific human disease or condition, such as cell surface and soluble receptor proteins, such as lymphocyte cell surface receptors, enzymes, such as proteases (serine, cysteine and acid) and thymidylate synthetase, steroid receptors, nuclear proteins, allosteric enzymes, clotting factors, kinases (both serine and threonine) and dephosphorylases (or phosphatases, either serine/threonine or protein tyrosine phosphatases e.g. PTP's especially PTP1B), bacterial enzymes, fungal enzymes and viral proteins or enzymes (especially those associated with HIV, influenza, rhinovirus and RSV), signal transduction molecules, transcription factors, proteins associated with DNA and/or RNA synthesis or degradation, immunoglobulins, hormones, receptors for various cytokines including, for example, erythropoietin (EPO), granulocyte colony stimulating (G-CSF) receptor, granulocyte macrophage colony stimulating (GM-CSF) receptor, thrombopoietin (TPO), interleukins, e.g. IL-2, IL-3, IL-4, IL-5, IL-6, IL-10, IL-11, IL-12, growth hormone, prolactin, human placental lactogen (LPL), CNTF, oncostatin, various chemokines and their receptors, such as RANTES MIPβ, IL-8, various ligands and receptors for tyrosine kinase, such as insulin, insulin-like growth factor 1 (IGF-1), epidermal growth factor (RGF), heregulin-α and heregulin-β, vascular endothelial growth factor (VEGF), placental growth factor (PLGF), tissue growth factors (TGF-α and TGF-β), nerve growth factor (NGF), various neurotrophins and their ligands, other hormones and receptors such as, bone morphogenic factors, follicle stimulating hormone (FSH), and luteinizing hormone (LH), thimeric hormones including tissue necrosis factor (TNF) and CD 40 ligand, apoptosis factor-1 and -2 (AP-1 and AP-2), mdm2, caspases, and proteins and receptors that share 20% or more sequence identity to these.

The target, e.g. a TBM of interest will be chosen such that it possesses, or is modified to possess, a reactive group which is capable of forming a covalent bond with a ligand having intrinsic affinity for a site of interest on the target. For example, many targets naturally possess reactive groups (for example, amine, thiol, aldehyde, ketone, hydroxyl groups, and the like) to which ligands, such as members of an organic small molecule library, may covalently bond. For example, polypeptides often have amino acids with chemically reactive side chains (e.g., cysteine, lysine, arginine, and the like). Additionally, synthetic technology presently allows the synthesis of biological target molecules using, for example, automated peptide or nucleic acid synthesizers, which possess chemically reactive groups at predetermined sites of interest. As such, a chemically reactive group may be synthetically introduced into the target, e.g. a TBM, during automated synthesis.

In one particular embodiment, the target comprises at least a first reactive group which, if the target is a polypeptide, may or may not be associated with a cysteine residue of that polypeptide, and preferably is associated with a cysteine residue of the polypeptide, if the tether chosen is a free or protected thiol group (see below). The target preferably contains, or is modified to contain, only a limited number of free or protected thiol groups, preferably nor more than about 5 thiol groups, more preferably no more than about 2 thiol groups, more preferably no more than one free thiol group, although polypeptides having more free thiol groups will also find use. The target, such as TBM, of interest may be initially obtained or selected such that it already possesses the desired number of thiol groups, or may be modified to possess the desired number of thiol groups.

When the target is a polynucleotide, a tether can be attached to the polynucleotide on a base at any exocyclic amine or any vinyl carbon, such as the 5- or 6-position of pyrimidines, 8- or 2-positions of purines, at the 5′ or 3′ carbons, at the sugar phosphate backbone, or at internucleotide phosphorus atoms. However, a tether and be introduced also at other positions, such as the 5-position of thymidine or uracil. In the case of a double-stranded DNA, for example, a tether can be located in a major or minor groove, close to the site of interest, but not so close as to result in steric hindrance, which might interfere with binding of the ligand to the target at the site of interest.

Those skilled in the art are well aware of various recombinant, chemical, synthesis and/or other techniques that can be routinely employed to modify a target, e.g. a polypeptide of interest such that it possesses a desired number of free thiol groups that are available for covalent binding to a ligand candidate comprising a free thiol group. Such techniques include, for example, site-directed mutagenesis of the nucleic acid sequence encoding the target polypeptide such that it encodes a polypeptide with a different number of cysteine residues. Particularly preferred is site-directed mutagenesis using polymerase chain reaction (PCR) amplification (see, for example, U.S. Pat. No. 4,683,195 issued 28 Jul. 1987; and Current Protocols In Molecular Biology, Chapter 15 (Ausubel et al., ed., 1991)). Other site-directed mutagenesis techniques are also well known in the art and are described, for example, in the following publications: Current Protocols In Molecular Biology, Ausuben et al., eds., 1991, Chapter 8; Molecular Cloning: A Laboratory Manual., 2^{nd} edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired variant.

Sources of new reactive groups, e.g. cysteines can be placed anywhere within the target. For example, if a cysteine is introduced onto the surface of the protein in an area known to be important for protein-protein interactions, small molecules can be selected that bind to and block this surface. The following section exemplifies target biological molecules that can be modified by the methods described herein.

Tables of Targets

| Immunology | Indications |
|---|---|
| IL-6 | Inflammation |
| B7/CD28 | Graft rejection |
| CD4 | Immunosuppression |
| CD3 | Immunosuppression |
| CD2 | Renal Transplantation |
| c-maf | Inflammation/Immunosuppression |
| CD11a/LFA1 (ICAM) | Immunosuppression/Inflammation |

| Enzymes | Indications |
|---|---|
| Phospholipase A2 | Inflammation |
| ZAP-70 | Immunosuppression |
| Phophodiesterase IV | Asthma |
| Interleukin converting enzyme (ICE) | Inflammation |
| Inosine monophosphate dehydrogenase | Autoimmune diseases |
| Tryptase | Psoriasis/asthma |
| CDK4 | Cancer |
| mTOR | Immunosuppression |
| PARP (Cell death pathway) | Stroke |
| Phosphatases | Cancer |
| Raf | Cancer |
| JNK3 | Neurodegeneration |
| MEK | Cancer |
| GSK-3 | Diabetes |
| FABI (Fatty acid biosynthesis) | Bacterial |
| FABH (Fatty acid biosynthesis) | Bacterial |
| BACE | Alzheimer's |
| IkB-ubiquitin Ligase | Inflammation/diabetes |
| Lysophosphatidic acid acetlytransferase | |
| CD26 (dipeptidyl peptidase IV) | |
| Akt | |
| TNF converting enzyme | Inflammation |

| Viral Targets | Indications |
|---|---|
| Rhinovirus protease | Common cold |
| Parainfluenza neuraminidase | Colds/Veterinary uses |
| HIV fusion gp41 | HIV infection/treatment |
| Hepatitis C Helicase | Hepatitis |
| Hepatitis C protease | Hepatitis |

| Protein-Protein Targets | Indications |
|---|---|
| ErbB Receptors | Cancer |
| Neurokinin-1 | Inflammation, Migraine |
| IL-9 | Asthma |
| FGF | Angiogenesis |
| PDGF | Angiogenesis |
| TIE2 | Angiogenesis |
| NFκB Dimerization | Inflammation |
| Tissue Factor/Factor VII | Cardiovascular Disease |
| Selectins | Inflammation |
| TGF-α | Angiogenesis |
| Angiopoietin I | Angiogenesis |
| APAF-1/Caspase 9 CARD | Stroke |
| Bcl-2 | Cancer |

| 7-Transmembrane | Indications |
|---|---|
| IL-8 | Stroke, inflammation |
| Rantes | Inflammation, Migraine |
| CC Chemokine Receptors | Asthma |
| GPR14/Urotensin | Angiogenesis |
| Orexin/Receptor | Appetite |
| C5a receptor | Sepsis/crohn's disease |
| Histamine H3 receptor | Allergy |
| CCR5 | HIV attachment |

| Target | PDB Codes | Accession No. | Crystal Structure Ref. |
|---|---|---|---|
| BACE | 1FKN | GB AAF13715 | Hong, L. et al., Science. 290(5489): 150-3 |
| | | | (2000). |
| Caspase 1 | 1BMQ | SWS P29466 | Okamoto, Y., et al., Chem Pharm Bull |
| | | | (Tokyo), 47(1): 11-21 (1999). |
| Caspase 4 | none | SWS P49662 | NA |
| Caspase 5 | none | SWS P51878 | NA |
| Caspase 3 | 1CP3 | SWS P42574 | Mittl, P R, et al., J Biol |
| | | | Chem, 272(10): 6539-47(1997). |
| Caspase 8 | 1I4E, 1QTN | SWS P08160; | Xu, G., et al., Nature, 410(6827): 494-7 |
| | | GB BAB32555 | (2001). |
| Caspase 9 | 3YGS | SWS P55211 | Qin, H., et al., Nature, 399(6736): 549-57 |
| | | | (1999). |
| RHV Prot | 1CQQ | SWS P04936 | Matthews, D., et al., 96(20): 11000-7 |
| | | | (1999). |
| Cathepsin K | 1MEM | SWS P43235 | McGrath, M E, et al., Nat Struct |
| | | | Biol, 4(2): 105-(1997). |
| Cathepsin S | 1BXF | SWS P25774 | Fengler, A., et al., Protein |
| | (model) | | Eng, 11(11): 1007-13(1998). |
| Tryptase | 1A0L | SWS P20231 | Pereira, P. J. et al., Nature, |
| | | | 392(6673): 306-11 (1998). |
| HCV Prot | 1A1R, | SWS Q81755 | Di Marco, et al., J Biol Chem. |
| | 1DY9 | | 275(10): 7152-7(2000). |
| CD26 | none | SWS P27487 | NA |
| TACE | 1BKC | GB U69612 | Maskos, K., et al., PNAS, 95(7): 3408-12 |
| | | | (1998). |
| ZAP-70 | none | SWS P43403 | NA |
| p38 MAP | 1P38 | SWS P47811 | Wang, Z., et al., PNAS, 94(6): 2327-32 |
| | | | (1997). |
| CDK-4 | none | SWS Q9XTB6 | NA |
| c-jun kinase | NA | SWS P45983 (C- | NA |
| | | Jun Kinase-1) | |
| | NA | SWS P45984 (C- | NA |
| | | Jun Kinase-2) | |
| | 1JNK | SWS P53779 (C- | NA |
| | | Jun Kinase-3) | |
| GSK-3 | NA | SWS P49840 | NA |
| | | (GSK-3A) | |
| | NA | SWS P49841 | NA |
| | | (GSK-3B) | |
| AKT | none | SWS P31749 | NA |
| MEK | none | SWS Q02750 | NA |
| Raf | none | SWS P04049 | NA |
| TIE-2 | none | SWS Q02763 | NA |
| ILK | none | SWS Q13418 | NA |
| IkB | NA | SWS O15111 | NA |
| | | (IKappaBKinase) | |
| | NA | SWS O14920 | NA |
| | | (IKappBKinBeta) | |
| Jak1 | none | SWS P23458 | NA |
| Jak2 | none | SWS O60674 | NA |
| Jak3 | none | SWS P52333 | NA |
| Tyk2 | none | SWS P29597 | NA |
| EGF Kinase | see Vasc. | | |
| | Endo. | | |
| | Growth | | |
| | Factor | | |
| | Receptor | | |
| | (VEGFR) | | |
| | and EGFR | | |
| | both with | | |
| | tyrosine | | |
| | kinase | | |
| | activity(Below): | | |
| VEGFR2/KD | NA | SWS P35968 | NA |
| R Kinase | | | |
| EGFR | NA | SWS P00533 | NA |
| TC-PTP | NA | SWS P17706 | NA: T-cell Protein Tyrosine Phosphatase |
| CDC25A | NA | SWS P30304 | NA |
| CDC25A | NA | GB O14757 | NA |
| CDK | | (CHK1) | |
| CD45 | NA | SWS P08575 | NA |
| PTP alpha | NA | SWS P18433 | NA |
| pol III | NA | SWS O14802 | NA; DNA directed RNA polymerase III |
| (PolRIIIA) | | | (?) |
| mur-D Ligase | NA | GB O14802 | NA |
| | | (E. coli) | |
| | NA | SWS P14900 | NA |
| | | (E. Coli) | |
| SHP | NA | SWS Q15466 | NA |
| PTP-1B | 1PTP | SWS P00760 | Finer-Moore, J S, et al., |
| | | | Proteins, 12(3): 203-22(1992). |
| SHIP-2 | none | SWS Q9R1V2 | NA |
| MEKK-1 | NA | SWS Q13233 | NA |
| PAK-1 | NA | SWS Q13153 | NA |
| ICAM-1 | NA | SWS P05362 | Bella, J., et al., Proc Natl Acad Sci USA, |
| | | | 95(8): 4140-5 (1998). |
| CD11A/LFA-1 | NA | SWS P20701 | Qu, A., et al., Proc Natl Acad Sci USA, |
| | | | 92(22): 10277-81 (1995). |
| TAF1 | UNSURE | UNSURE (see | UNSURE |
| | | below) | |
| | NA | SWS Q99142 (?? | NA; tobacco Tumor Activating Factor (?) |
| | | Tobacco Prot.) | |
| | NA | GB AAB30018 | NA; Tumor-derived Adhesion Factor (?) |
| | NA | GB D45198 | NA; Template Activating Factor (?) |
| HIV-Integrase | 1BL3 (2.0) | SWS P12497 | Maignan, S., et al., J Mol Biol, |
| | | | 282(2): 359-68 (1998). |
| | 1EXQ | SWS P04585 | Chen, J. C-H., et al., PNAS USA, |
| | | | 97(15): 8233-8 (2000). |
| | NA | SWS O56380 | NA |
| | 1HYZ | SWS O56381; | Molteni, V., et al., Acta Crystallogr |
| | | GB AAC37875 | |
| | | | D Bio Crystallog., 57: 536-44 (2001). |
| | 1HYV | GB AAC37875 | Molteni, V., et al., Acta Crystallogr |
| | | | D Biol Crystallogr., 57(Pt 4): 536-44 |
| | | | (2001). |
| | NA | SWS O56382 | NA |
| | NA | SWS O56383 | NA |
| | NA | SWS O56384 | NA |
| | NA | SWS O56385 | NA |
| HCV-Helicase | 1N13, | SWS Q81755 | Di Marco, S., et al., J Biol Chem., |
| | 1DY9, | (1DY9) | 275(10): 7152-7 (2000). |
| | others | (Integrase) | |
| | 1HEI | SWS P2664 | Yao, N., et al., Nat Struct Biol, 4(6): 463- |
| | | (Helicase) | 7 (1997). |
| Infl. | 1A4G; | SWS P27907 | Taylor, N., et al., J Med Chem, |
| Neuraminidase | many | | 41(6): 798-807 (1998). |
| PDE-IV | 1FOJ | SWS Q07343 | Xu, R. X., et al., Science., |
| | (PDE4B2B) | | 288(5472): 1822-5 (2000). |
| cPLA-2 | 1CJY | SWS P47712 | Dessen, A., et al., Cell., 97(3): 349-60 |
| | | | (1999). |
| IL-2 | NA (in- | SWS P01585 | NA |
| | house) | | |
| IL-4 | 1HIK(apo) | SWS P05112 | Muller, T., et al., J Mol Biol, 247(2): 360- |
| | | (2.60) | 72 (1995). |
| | 1IAR | SWS P05112 | Hage, T., et al., Cell., 97(2): 271-81 |
| | (complex) | (2.30) ** | (1999). |
| IL-4R | 1IAR | SWS P24394 | Hage, T., et al., Cell., 97(2): 271-81 |
| | | | (1999). |
| IL-5 | 1HUL | SWS P05113 | Milburn, M. V., et al., Nature, |
| | | | 363(6425): 172-6 (1993). |
| IL-6 | 1I1R(viral | GB AAB62676 | Chow, D., et al., Science, |
| | IL6) | (2.6) | 291(5511): 2150-5 (2001). |
| | 1ALU | SWS P05231 | Somers, W., et al., EMBO J, 16(5): 989- |
| | | (1.9) | 97 (1997). |
| IL-7 | 1IL7 | SWS P13232 | Cosenza, L., et al., Protein Sci., 9(5): 916- |
| | (model) | | 26 (2000). |
| IL-9 | none | SWS P15248 | NA |
| IL-13 | 1GA3 | SWS P35225 | NA |
| | (NMR) | | |
| TNF | 1TNF | SWS P01375 | Eck, M J, et al., J Biol |
| | | (TNF-alpha) | Chem, 264(29)17595-605(1989). |
| CD-40 L | 1ALY | SWS P29965 | Karpusas, M., et al., Structure, |
| | | | 3(12): 1426 (1995). |
| OPGL | none | SWS O14788 | NA |
| BAFF | none | SWS Q9Y275 | NA |
| TRAIL | 1DG6 (1.30) | GB AAC50332 | Hymowitz, S. G., et al., Biochemistry, |
| | | | 39(4): 633-40 (2000). |
| | 1DU3 (2.2) | SWS P50591; | Cha S S, et al., J Biol Chem, |
| | | GB AAC50332 | 275(40): 31171-7 (2000). |
| | 1D2Q | GB AAC50332 | Cha and Oh, Immunity, 11(2): 253-61 |
| | | | (1999). |
| IL-1 | NA | SWS P01584 | NA |
| | | (IL-1 B | |
| | | Cytokine) | |
| IL-1R | 1G0Y | SWS P14778 | Vigers, GPA, et al., J Biol Chem., |
| | | | 275(47): 36927-33 (2000). |
| IL-8 | 1QE6 | SWS P10145 | Gerber, N., et al., Proteins, 38(4): 361-7 |
| | | | (2000). |
| RANTES-R | NA | SWS P32246 | NA |
| RANTES | NA | GB XP_035842 | NA |
| | NA | SWS P13501 | NA; (?? T-cell specific RANTES protein) |
| MCP-1 | NA | SWS Q14805 | NA; (?? Metaphase chromosomal |
| | | | protein) |
| MCP-1 | 1D0K | SWS P13500 | Lubowski, J., et al., Nat Struct Biol., |
| | | | 4(1): 64-9 (1997). |
| MCP-3 | NA | SWS P80098 | Nat Struct Biol, 4(1): 64-9 (1997). |
| TRAF-A | NA | SWS Q13077 | NA |
| (TRAF-1?) | | (TRAF-1) | |
| TRAF-B | NA | SWS Q12933 | NA |
| (TRAF-2?) | | (TRAF-2) | |
| | 1D00 | GB S56163 | Ye, H., et al., Mol Cell, 4(3): 321-30 |
| | (TRAF-2) | (TRAF-2) (2.0) | (1999). |
| TRAF-C | NA | SWS Q13114 | NA |
| (TRAF-3?) | | (TRAF-3) | |
| TRAF-D | NA | GB XP_008483 | NA |
| (TRAF-4?) | | (TRAF-4) | |
| TRAF-E | NA | GB XP_010656 | NA |
| (TRAF-5?) | | (TRAF-5) | |
| VEGF | 1FLT | SWS P15692 | Wiesmann, C., et al., Cell, 91(5): 695-704 |
| | | | (1997). |
| Mineral | NA | SWS P08235 | NA |
| Corticoid R. | | | |
| Estrogen | 3ERD | SWS P03372 | Shiau, A. K., Barstad, D., Loria, P. M., |
| Receptor | | | Cheng, L., Kushner, P. J., Agard, D. A., |
| | | | Greene, G. L., Cell, 95(7): 927-37 (1998). |
| Progesterone | 1A28 | SWS P06401 | Williams, S. P., Sigler, P. B, Nature, |
| Rec. | | | 393(6683): 392-6 (1998). |
| NF-kappa-B-1 | | SWS P19838 | |
| P53 | NA | SWS P04637 | NA |
| | Y1CQ | GB AAA59989 | Kussie, P. H., et al., |
| | | (2.3) | Science, 74(5289): 948-53 (1996). |
| MDM2 | 1YCR | SWS Q00987 | Kussie, P. H., et al., |
| | | | Science, 74(5289): 948-53 (1996). |
| STAT6 | NA | SWS P42226 | NA |
| IL4R-alpha | NA | SWS P24394 | NA |
| IL6R-alpha | NA | SWS P08887 | NA |
| IL6R-beta | 1BQU | SWS P40189 | Bravo, J., Staunton, D., Heath, J. K., |
| chain | | | Jones, E. Y., EMBO J, 17(6): 1665-74 |
| | | | (1998). |
| IL5R-alpha | NA | SWS Q01344 | NA |
| IL7R | NA | SWS P16871 | NA |
| IL2R-alpha | NA | SWS P01589 | NA |
| IL2R-beta | NA | SWS P14784 | NA |
| HIV GP41 | 1AIK | SWS P19551 | Chan, D. C., Fass, D., Berger, J. M., Kim, |
| | | | P. S., Cell, 89(2): 263-73 (1997). |
| HIV GP41 | 1AIK | SWS P04582 | Chan, D. C., Fass, D., Berger, J. M., Kim, |
| | | | P. S., Cell, 89(2): 263-73 (1997). |
| HIV GP41 | | SWS P03378 | |
| HIV GP41 | | SWS P03375 | |
| HIV GP41 | | SWS P04582 | |
| HIV GP41 | | SWS P12488 | |
| HIV GP41 | | SWS P03377 | |
| HIV GP41 | | SWS P05879 | |
| HIV GP41 | | SWS P04581 | |
| HIV GP41 | | SWS P04578 | |
| HIV GP41 | | SWS P04624 | |
| HIV GP41 | | SWS P12489 | |
| HIV GP41 | | SWS P20871 | |
| HIV GP41 | | SWS P31819 | |
| HIV GP41 | | SWS Q70626 | |
| HIV GP41 | | SWS P04583 | |
| HIV GP41 | | SWS P19551 | |
| HIV GP41 | | SWS P05577 | |
| HIV GP41 | | SWS P18799 | |
| HIV GP41 | | SWS P20888 | |
| HIV GP41 | | SWS P03376 | |
| HIV GP41 | | SWS P04579 | |
| HIV GP41 | | SWS P19550 | |
| HIV GP41 | | SWS P19549 | |
| HIV GP41 | | SWS P05878 | |
| HIV GP41 | | SWS P31872 | |
| HIV GP41 | | SWS P05880 | |
| HIV GP41 | | SWS P35961 | |
| HIV GP41 | | SWS P12487 | |
| HIV GP41 | | SWS P04580 | |
| HIV GP41 | | SWS P05882 | |
| HIV GP41 | | SWS P05881 | |
| HIV GP41 | | SWS P18094 | |
| HIV GP41 | | SWS P24105 | |
| HIV GP41 | | SWS P17755 | |
| HIV GP41 | | SWS P15831 | |
| HIV GP41 | | SWS P18040 | |
| HIV GP41 | | SWS Q74126 | |
| HIV GP41 | | SWS P05883 | |
| HIV GP41 | | SWS P04577 | |
| HIV GP41 | | SWS P32536 | |
| HIV GP41 | | SWS P12449 | |
| HIV GP41 | | SWS P20872 | |
| c-mal | NA | GB NP_071884 | NA; T-cell differentiation protein (?) |
| | NA | GB CAA54102 | NA |
| | NA | GB XP_017128 | NA |
| Mal | NA | SWS P21145 | NA; T-LYMPHOCYTE |
| | | | MATURATION-ASSOCIATED |
| | | | PROTEIN |
| | NA | SWS P01732 | NA; T-LYMPHOCYTE |
| | | | DIFFERENTIATION ANTIGEN |
| | | | T8/CD8(?) |
| Her-1 | NA | SWS P34704 | NA: Cell Signaling in C. elegans Sex |
| | | | Determination (?) |
| Her-2 | NA | SWS P04626 | NA; RECEPTOR PROTEIN- |
| | | | TYROSINE KINASE ERBB-2 (?) |
| E2F-1 | NA | SWS Q01094 | NA |
| E2F-2 | NA | SWS Q14209 | NA |
| E2F-3 | NA | SWS O00716 | NA |
| E2F-4 | NA | SWS Q16254 | NA |
| E2F-5 | NA | SWS Q15329 | NA |
| E2F-6 | NA | SWS O75461 | NA |
| Cyclin A | 1QMZ | SWS P20248 | Brown, N. R., et al., Nat Cell Biol., |
| | | | 1(7): 438-43 (1999). |
| mTOR/FRAP | 1NSG | SWS P42345 | Liang, J., et al., Acta Crystall D Biol |
| | | | Crystall, 55 (Pt 4): 736-44 (1995). |
| Survivin | 1F3H | SWS O15392 | Verdecia, M. A., et al., Nat Struct Biol., |
| | | | 7(7): 602-8 (2000). |
| FGF-1 | 1EV2 | SWS P05230 | Plotnikov, A. N., et al., Cell., |
| | | | 101(4): 413-24 (2000).(Heparin Binding |
| | | | Growth Factor I) |
| Basic FGF | 1FGK | SWS P11362 | Mohammadi, M., et al., Cell, 86(4): 577- |
| Rec. I | | | 87 (1996). (Basic FGF Rec. I) |
| FGF-2 | 1CVS | SWS P09038 | Plotnikov, A. N., et al., Cell, 98(5): 641- |
| | | | 50 (1999). |
| FGF-3 | NA | SWS P11487 | NA |
| FGF-4 | NA | SWS P08620 | NA |
| FGF-5 | NA | SWS P12034 | NA |
| FGF-6 | NA | SWS P10767 | NA |
| FGF-7 | NA | SWS P21781 | NA |
| FGF-8 | NA | SWS P55075 | NA |
| FGF-9 | 1IHK | SWS P31371 | Plotnikov, A. N., et al., J Biol Chem., |
| | | | 276(6): 4322-9 (2001). |
| PARP | NA | SWS P09874 | NA |
| PDGF-alpha | NA | SWS P04085 | NA |
| PDGF-beta | NA | SWS P01127 | NA |
| C5a receptor | NA | SWS P21730 | NA |
| CCR5 | NA | SWS P51681(CC | NA |
| | | Chemo R-V) | |
| GPR14/Urote- | NA | SWS Q9UKP6 | NA |
| nsin IIR) | | | |
| Tissue Factor | 2HFT | SWS P13726 | Muller, Y. A., et al., J Mol Biol, |
| | | | 256(1): 144-59 (1996). |
| Factor VII | 1JBU | SWS P08709 | Eigenbrot, C., et al., Structure, 9: 627 |
| | | | (2001). |
| Histamine H3 | NA | GB CAC39434 | NA |
| rec. | | | |
| Neurokinin-1 | NA | GB SPHUB | NA |
| orexin | NA | SWS O43613 | NA |
| receptor-1 | | | |
| orexin | NA | SWW O43614 | NA |
| receptor-2 | | | |
| CD-3 delta | NA | SWS P04234 | NA |
| chain | | | |
| CD-3 epsilon | NA | SWS P07766 | NA |
| chain | | | |
| CD-3 gamma | NA | SWS P09693 | NA |
| chain | | | |
| CD-3 zeta | NA | SWS P20963 | NA |
| chain | | | |
| CD-4 | 1CDJ | SWS P01730 | Wu, H., et al., Proc Natl Acad Sci USA, |
| | | | 93(26): 15030-5 (1996). |
| TGF-alpha | NA | SWS P01135 | NA |
| TGF-beta-1 | NA | SWS P01137 | NA |
| TGF-beta-2 | NA | SWS P08112 | NA |
| TGF-beta-3 | NA | SWS P10600 | NA |
| TGF-beta-4 | NA | SWS O00292 | NA |
| GRB2 | 1GRI | SWS P29354 | Maignan S, et al., Science, |
| | | (3.1) | 268(5208): 291-3 (1995). |
| | 1ZFP | SWS P29354 | Rahuel, J., et al., J Mol Biol, |
| | | (1.8) | 279(4): 1013-22 (1998). |
| | 1BMB | SWS P29354 | Ettmayer, P., et al., J Med Chem, |
| | | (1.8) | 42(6): 971-80 (1999). |
| LCK | 1LKK | SWS P06239; | Tong, L., et al., J Mol Biol, 256(3): 601- |
| | | (2nd = P07100) | 10 (1996). |
| SRC | 2SRC | SWS P12931 | Xu, W., et al., Mol Cell., 3(5): 629-38 |
| | | | (1999). |
| TRAFs ? | NA | SWS Q13077 | NA |
| | | (TRAF-1) | |
| | 1CZZ | GB S56163 | Ye, H., et al., Mol Cell, 4(3): 321-30 |
| | (TRAF-2) | (TRAF-2) (2.7) | (1999). |
| | 1CZY | GB S56163 | Ye, H., et al., Mol Cell, 4(3): 321-30 |
| | (TRAF-2) | (TRAF-2) (2.0) | (1999). |
| | 1D00 | GB S56163 | Ye, H., et al., Mol Cell, 4(3): 321-30 |
| | (TRAF-2) | (TRAF-2) (2.0) | (1999). |
| | NA | SWS Q12933 | NA |
| | | (TRAF-2) | |
| | 1FLK | GB Q13114 | Ni, C.-Z., et al., Proc Natl Acad Sci USA., |
| | (TRAF-3) | (TRAF-3) (2.8) | 97(19): 10395-9 (2000). |
| BAX/BCL-2 | NA | SWS Q07812 | NA |
| | | (BAX alpha) | |
| | NA | SWS Q07814 | NA |
| | | (BAX beta) | |
| | NA | SWS Q07815 | NA |
| | | (BAX gamma) | |
| | NA | SWS P55269 | NA |
| | | (BAX delta) | |
| | NA | SWS P10415 | NA |
| | | (BCL-2) | |
| IgE | 1F6A (3.5) | SWS P01854 | Garman, S. C., et al., T. S., Nature., |
| | | (IgE chain C) | 406(6793): 259-66 (2000). |
| IgER | NA | SWS P06734 | NA |
| | | (IgE Fc | |
| | | Receptor) | |
| | 1F6A (3.5) | SWS P12319 | Garman, S. C., et al., T. S., Nature., |
| | | (IgE Fc Rec. | 406(6793): 259-66 (2000). |
| | | alpha) | |
| | 1F2Q (2.4) | SWS P12319 | Garman, S. C., Kinet, J. P., Jardetzky, T. |
| | | (IgE Fc Rec. | S., Cell, 95(7): 951-61 (1998). |
| | | alpha) | |
| | NA | SWS Q01362 | NA |
| | | (IgE FcRec. | |
| | | Beta) | |
| | NA | SWS P30273 | NA |
| | | (IgE FcRec. | |
| | | Gama) | |
| Rhinovirus | NA | SWS P03303 | NA |
| Protease | | (HRV-14 | |
| | | polyprot.) | |
| | NA | SWS P12916 | NA |
| | | (HRV-1B) | |
| | 1CQQ | SWS P04936 | Matthews, D., et al., Proc Natl Acad Sci |
| | | (HRV-2) (1.85) | USA, 96(20): 11000-7 (1999). |
| | NA | SWS P07210 | NA |
| | | (HRV-89) | |
| | 1C8M | SWS Q82122 | Chakravarty, S., et al., to be published |
| | | (HRV-16) | |
| B7/CD28LG/ | 1DR9 | SWS P33681 | Ikemizu, S., et al., Immunity. 2000 |
| CD80 | | | Jan; 12(1): 51-60. |
| CD28 | NA | SWS P10747 | NA |
| APAF1 | NA | SWS O14727 | NA |

Choice of Tethers and Reversible Covalent Bonds

As described previously, TBM variants are produced so that they can be used to link ligands, directly or indirectly, through a tether, forming a reversible covalent bond. The tether attaches a ligand having intrinsic affinity for the target to the target either by forming a reversible covalent bond between the target and the ligand, or between a tethered first ligand and a second ligand, near a site of interest on the target. In the latter case, the tether is formed by reacting the functional group on a small molecule extender, as hereinabove defined, with a compatible reactive group on a second ligand, having intrinsic affinity for a second site of interest on the target, e.g. a target biological molecule (TBM).

The tether on the target may be the same as or different from the reactive group present on the ligand candidate(s), such as a small organic molecules. The tether(s) should be of appropriate length and flexibility to ensure that the ligand candidates have free access to the site of interest on the target. While it is usually preferred that the attachment of the tether does not denature the target, the tether-target complex, e.g. the TBM-extender complex may also be formed under denaturing conditions, followed by refolding the complex by methods known in the art. Moreover, the tether and the reversible covalent bond should not substantially alter the three-dimensional structure of the target, so that the ligands will recognize and bind to a site of interest on the target with useful site specificity. Finally, the tether and the reversible covalent bond should be substantially unreactive with other sites on the target under the reaction and assay conditions used.

The tethers may, for example be free or protected thiol (—SH) groups (including activated thiol groups) that can be present or may be introduced into the target and the ligand candidates, such as TBM and small organic molecules. A reversible bond, such example a disulfide (—S—S—) or metal-bridged disulfide bond, formed between thiol groups can be broken by contacting the bond with a reducing agent. Thus, suitable reversible covalent bonds include, for example linkages represented by the formula L-SS-T (wherein L stands for ligand and T stands for target). Other reversible bonds include, for example, imines (se, e.g. Huc and Lahn, Proc. Natl. Acad. Sci. USA 94: 2106-2110 (1997), covalent bonds formed between primary or secondary amine groups, and alcohol (—OH), aldehyde or ketone groups. Also included are covalent bonds in which a metal (e.g. Fe³⁺, Co²⁺, Ni²⁺, Cu²⁺, Zn²⁺ Cd²⁺, or Hg²⁺) is complexed between a multidentate ligand, wherein the reactive group on the ligand can, for example, be S, N or an imidazole group, and a reactive moiety on the target, wherein the moiety on the target can be S, N or an imidazole group. Boronate esters may also serve as tethers.

For example, small organic molecules that may serve as targets, in particular target biological molecules (TBM's) in the methods described herein include aldehydes, ketones, oximes, such as O-alkyl oximes, preferably O-methyl oximes, hydrazones, semicarbazones, carbazides, primary amines, secondary amines, such as N-methylamines, tertiary amines, such as N,N-dimethyl amines, N-substituted hydrazines, hydrazides, alcohols, ethers, thiol, thioethers, thioesters, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epozides, aziridines, isocyatanes, sulfonyl chlorides, diazo compounds, acid chlorides, and the like, all of which have counterpart reactive groups that allow covalent bond formation with a polypeptide of interest, which may be appropriately modified, if necessary. In fact, virtually any small organic molecule that is capable of covalently bonding to a known chemically reactive functionality may be used as a tether, with the proviso that it is sufficiently soluble and stable in aqueous solutions to be tested for its ability to bind to the biological target molecule.

Those of ordinary skill in the art will be capable of covalently linking practically any reactive group on a ligand candidate to a moiety on the target by techniques known in the art.

Thus, various chemistries available for forming a reversible covalent bond between reactive groups on a ligand and a target, or between two ligands, are well known in the art, and are described in basic textbooks, such as, e.g. March, Advanced Organic Chemistry, John Wiley & Sons, New York, 4^{th} edition, 1992. Reductive aminations between aldehydes and ketones and amines are described, for example, in March et al., supra, at pp. 898-900; alternative methods for preparing amines at page 1276; reactions between aldehydes and ketones and hydrazide derivatives to give hydrazones and hydrazone derivatives such as semicarbazones at pp. 904-906; amide bond formation at p. 1275; formation of ureas at p. 1299; formation of thiocarbamates at p. 892; formation of carbamates at p. 1280; formation of sulfonamides at p. 1296; formation of thioethers at p. 1297; formation of disulfides at p. 1284; formation of ethers at p. 1285; formation of esters at p. 1281; additions to epoxides at p. 368; additions to aziridines at p. 368; formation of acetals and ketals at p. 1269; formation of carbonates at p. 392; formation of denamines at p. 1264; metathesis of alkenes at pp. 1146-1148 (see also Grubbs et al., Acc, Chem. Res. 28:446-453 [1995]); transition metal-catalyzed couplings of aryl halides and sulfonates with alkanes and acetylenes, e.g. Heck reactions, at p.p. 717-178; the reaction of aryl halides and sulfonates with organometallic reagents, such as organoboron, reagents, at p. 662 (see also Miyaura et al., Chem. Rev. 95:2457 [1995]); organotin, and organozinc reagents, formation of oxazolidines (Ede et al., Tetrahedron Letts. 28:7119-7122 [1997]); formation of thiazolidines (Patek et al., Tetrahedron Letts. 36:2227-2230 [1995[); amines linked through amidine groups by coupling amines through imidoesters (Davies et al., Canadian J. Biochem. c50:416-422 [1972]), and the like. In particular, disulfide-containing small molecule libraries may be made from commercially available carboxylic acids and protected cysteamine (e.g. mono-BOC-cysteamine) by adapting the method of Parlow et al., Mol. Diversity 1:266-269 (1995), and can be screened for binding to polypeptides that contain, or have been modified to contain, reactive cysteines. All of the references cited in this section are hereby expressly incorporated by reference.

Linker elements that find use for linking two or more organic molecule ligands to produce a conjugate molecule will be multifunctional, preferably bifunctional, cross-linking molecules that can function to covalently bond at least two organic molecules together via reactive functionalities possessed by those molecules. Linker elements will have at least two, and preferably only two, reactive functionalities that are available for bonding to at least two organic molecules, wherein those functionalities may appear anywhere on the linker, preferably at each end of the linker and wherein those functionalities may be the same or different depending upon whether the organic molecules to be linked have the same or different reactive functionalities. Linker elements that find use herein may be straight-chain, branched, aromatic, and the like, preferably straight chain, and will generally be at least about 2 atoms in length, more generally more than about 4 atoms in length, and sometimes as many as about 12 or more atoms in length. Linker elements will generally comprise carbon atoms, either hydrogen saturated or unsaturated, and therefore, may comprise alkanes, alkenes or alkynes, and/or other heteroatoms including nitrogen, sulfur, oxygen, and the like, which may be unsubstituted or substituted, preferably with alkyl, alkoxyl, hydroxyalkyl or hydroxyalkyl groups. Linker elements that find use will be of varying lengths, thereby providing a means for optimizing the binding properties of a conjugate ligand compound prepared therefrom. The first organic compound that covalently bound to the target biomolecule may itself possess a chemically reactive group that provides a site for bonding to a second organic compound. Alternatively, the first organic molecule may be modified (either chemically, by binding a compound comprising a chemically reactive group thereto, or otherwise) prior to screening against a second library of organic compounds.

Disulfide Ligands or Libraries for Screening a Target Containing a Free Thiol

Libraries of molecules of Formula I-XIV below are preferred for screening the Targents, mutants and variants of the present invention.

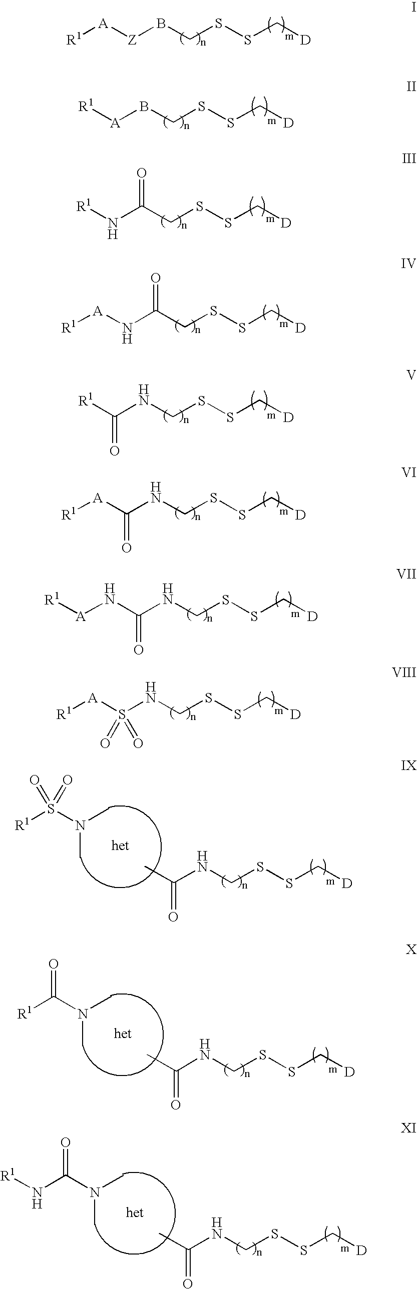

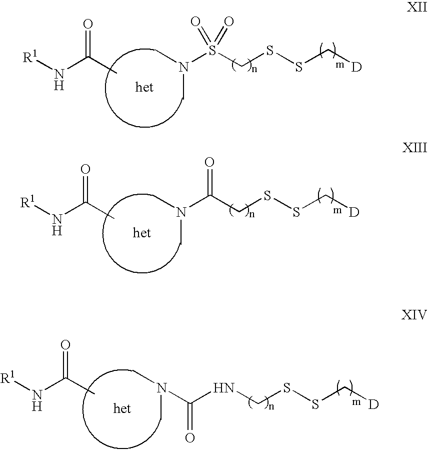

Where the substituents for these molecules are as defined above.

Construction of Disulfide Tethering Libraries Comprising Molecules of Formula I-XIV.

a. Preparation of Tethering Linkers.

Two exemplary linkers used in the preparation of tethering monophores, one bearing an amine and the other a carboxylate, are shown below:

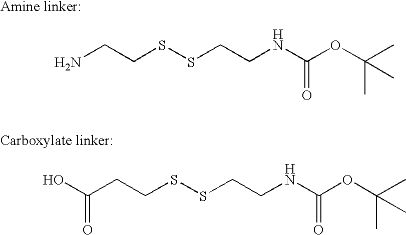

The amine linker is conveniently employed for building blocks bearing a carboxylate, sulfonylchloride or isocyanate, while the carboxylate linker is employed for the derivatization of amines. Other linkers exist in which the number of atoms between the disulfide and functional group are varied. For example, one can use an aminopropyl-derived disulfide linker, or a thioacetate linker and the like.

b. Carboxylic Acid Library (Library 1)

Carboxylic acid building blocks obtained from commercial sources are converted to tethering monophores following the scheme outlined below:

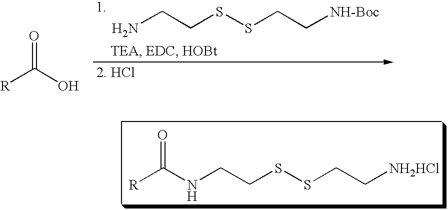

Reactions are performed in dichloromethane using an activating agent, such as EDC/HOBt. Other activating agents could be used, but EDC is highly water-soluble and facilitates aqueous work-up. Compounds are purified by a semi-automated aqueous work-up on a Tecan Genesis workstation (Tecan US, Inc., Durham, N.C.). Isolated products are deprotected with HCl/Dioxane, evaporated to dryness, and diluted with DMSO to a final molarity of 100 mM. Products may be used singly or may be pooled by combining them in pools of 9 to 12 members based upon a molecular weight pooling scheme that maximizes molecular weight differences. The resulting stock solutions are saved in vials and then aliquoted into microtiter plates for screening. All compounds are stored in −80 C freezer. Once a microtiter plate is thawed, it is stored at 4 C until consumed or discarded.

c. Arylamine Library (Library 2).

Anilines are less reactive than simple aliphatic amines so the chemistry is optimized prior to library synthesis. As outlined below, three representative aryl amines can be acylated with the carboxylate tethering linker using several different sets of conditions, including CDI/HOBt, -OpFp ester, HATU, EDC/HOBt chemistry, and the Vilsmeier reagent:

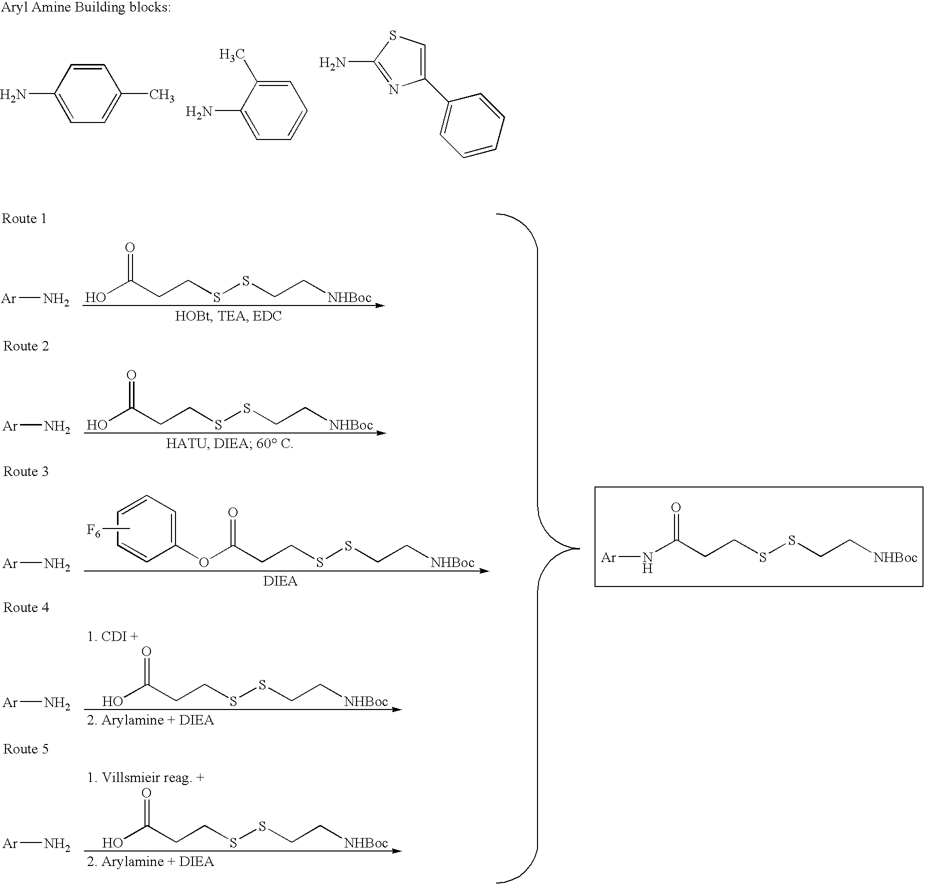

One preferred synthetic route employs Vilsmeier reagent for the synthesis of the intermediate acid chloride which is then added to a solution of the amine, and most libraries containing aryl amine building blocks are prepared using these conditions. Crude compounds are purified by either preparative reverse phase (C18) HPLC or aqueous-organic extraction, then deprotected with HCl/dioxane and processed as described above.

d. Sulfonamide and Urea Library (Library 3)

Isocyanates and sulfonylchlorides are available from commercial sources or can be made by standard procedures. Acylation and sulfonylation conditions using representative building blocks as shown below:

### Isocyanate and Sulfonyl Chloride Building Blocks:

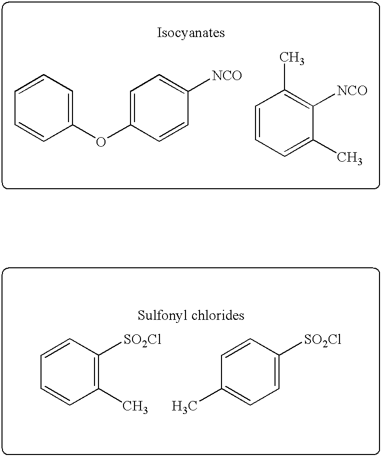

Synthesis Scheme:

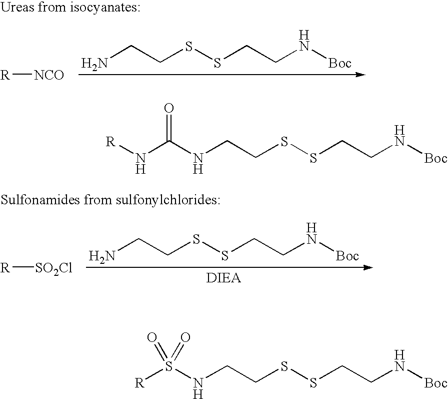

Reaction products are conveniently purified using a modified aqueous work-up on a TECAN GENESIS liquid handling workstation (Tecan US, Inc., Durham, N.C.), and purified products are deprotected as previously described.

e. Libraries Prepared from Aliphatic Amines

Aliphatic amines are readily acylated with the carboxylate-containing disulfide linker using EDC/HOBt conditions. Aqueous work-up and deprotection are as described above.

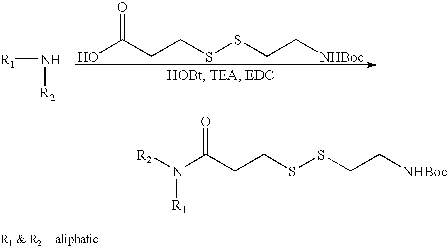

f. Libraries Prepared from Aminoacids

Side-chain protected and C-protected amino acids, such as alpha amino acids, beta-aminoacids, and the like, can be converted into tethering reagents using the simple EDC/HOBt procedure described above to acylate their free amine with the tethering linker. Unprotected amino acids require special conditions since their free carboxylate as well as some side-chain functionality can interfere with the coupling process. After some experimentation, it was found that unprotected amino acids could be acylated with the acid-chloride of the tethering linker in good overall yield. The acid-chloride was conveniently and mildly prepared using the Vilsmeier reagent. The activated linker is then added to a solution of the amino acid in a solvent such as DCM, in the presence of a base such as TEA. Other bases can be used, such as aqueous Na₂CO₃ and the like. Resulting products are purified either by HPLC or by a modified aqueous workup in which the products are only washed with aqueous acid. Deprotection then proceeds as described previously:

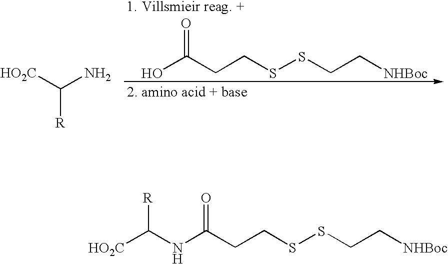

g. Libraries Prepared from Aldehydes and Ketones

Aldehydes and ketones react cleanly and stoichiometrically with an oxime tethering linker, and typically require no further purification. Deprotection proceeds as described previously. A scheme for their preparation is shown below:

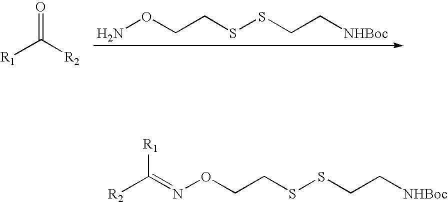

h. Synthesis of Two-Component Fragment Libraries.

Many of the tethered fragments described above can serve as substrates for additional chemical synthesis to create new fragments. For example, a reagent “A” (see scheme, below) can contain two or more functional groups (“X” and “Y”) suitable for performing synthetic organic chemistry. One functional group can be used to install the disulfide tether, while the others can be used to incorporate additional reagents “B” (see scheme).

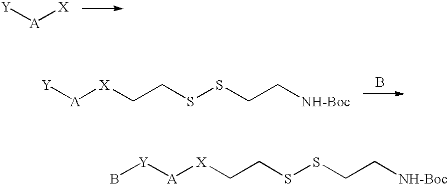

To illustrate, a protected amino acid can be coupled to a disulfide tether using simple amide bond chemistry, then the amine could be deprotected and further acylated to create a tethered dipeptide:

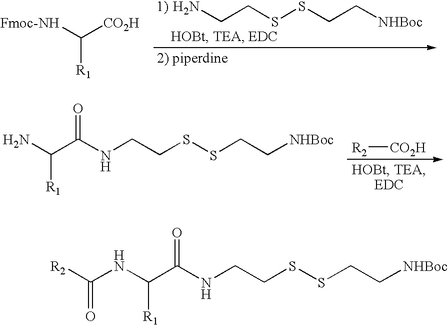

One skilled in the art will recognize that a number of transformations can be used in this manner to create libraries of disulfide-tethered fragments. Both solution phase and solid phase synthesis methods can be employed, as can powerful combinatorial synthesis techniques. For example, most of the transformations described in the combinatorial chemistry literature can be adapted for the preparation of disulfide tethering reagents (for examples see “The Combinatorial Index” by Barry Bunin). In some instances, certain reagents are not compatible with the presence of the disulfide tether; however, most chemistries can be adapted such that the disulfide tether is installed in the last synthetic step and thus eliminate any compatibility issues.

Examples of combinatorial chemistries that can be employed for the preparation of tethering reagents include the synthesis of heterocycles such as thiazolidinones, thiazoles, aminothiazoles, pyrrolidinones, thiadiazoles, triazoles, quinazolinediones, and the like. Palladium-mediated cross-couplings using the methods of Suzuki, Stille and Heck and others can be used for the preparation of biaryls, stilbenes and the like as tethering reagents. Peptidic compounds can be prepared using standard peptide synthesis conditions that are well-described in the literature, via submonomer synthesis methods, as well as via multicomponent condensations using the method of Ugi.

Libraries Produced by Derivatization of Constrained Amino Acid Analogs.

A general schematic for the N- and C-side modification of a constrained amino acid is illustrated in the figure, below:

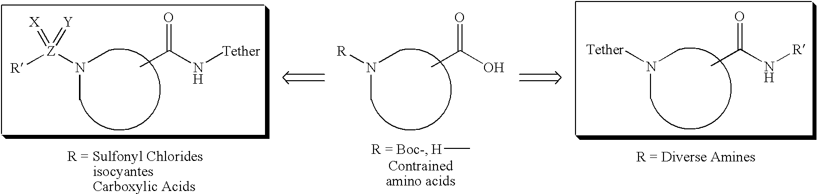

i. Selection of Amino Acids.

The ACD was examined to identify every commercially available cyclic aliphatic and aryl amino acid. Obvious choices included D- and L-proline, D- and L-pipicolinic acid, and related analogs (see figure, below).

j. Examples of Constrained Bifunctional Building Blocks:

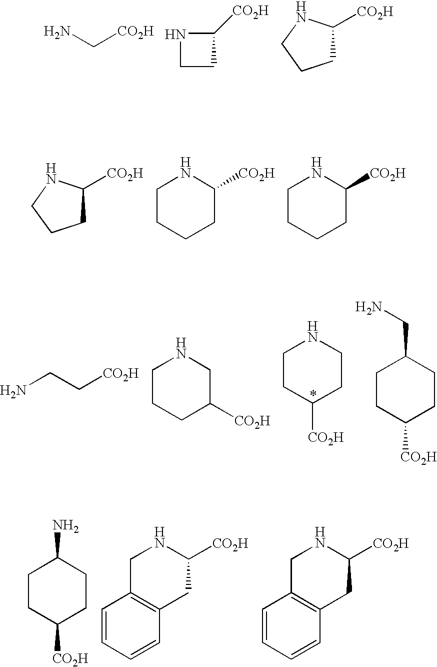

Other commercially-available constrained bifunctional amino acids include:

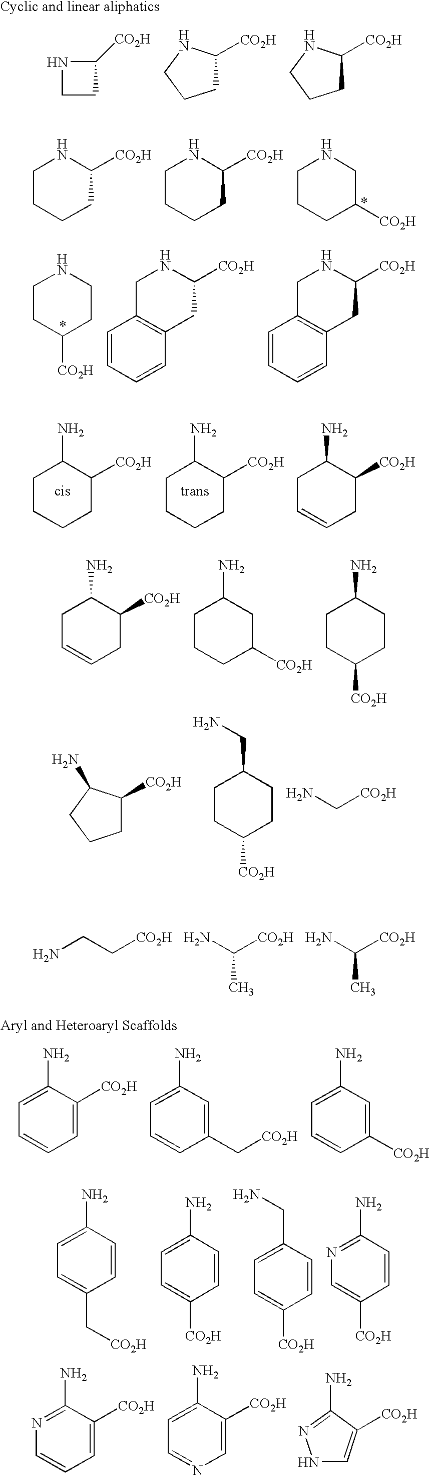

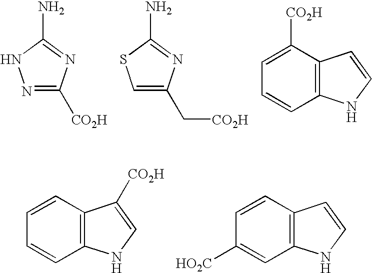

k. Examples of Constrained Tifunctional Building Blocks:

Trifunctional building blocks are also considered advantageous, since the additional point of modification can allow 1) the synthesis of additional regioisomers, 2) combinatorial elaboration/refinement of a monophore hit, and 3) a potential site for recombination with other monophore hits. The reagents trans-hydroxyproline, and R- and S-piperazine-2-carboxylic acid are commercially available as are the unconstrained amino acids D- and L-2,3-diaminoproprionic acid (DAP), Asn, Gln, and Tyr, as illustrated below.

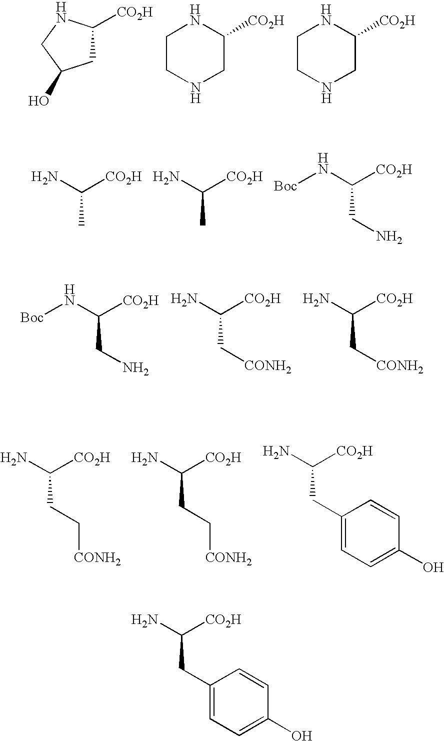

Detection and Identification of Ligands Bound to a Target

Following tethering the ligand to a TBM, the ligands bound to a target can be readily detected and identified by mass spectroscopy (MS). MS detects molecules based on mass-to-charge ratio (m/z) and thus can resolve molecules based on their sizes (reviewed in Yates, Trends Genet. 16: 5-8 [2000]). A mass spectrometer first converts molecules into gas-phase ions, then individual ions are separated on the basis of m/z ratios and are finally detected. A mass analyzer, which is an integral part of a mass spectrometer, uses a physical property (e.g. electric or magnetic fields, or time-of-flight [TOF]) to separate ions of a particular m/z value that then strikes the ion detector.

Mass spectrometers are capable of generating data quickly and thus have a great potential for high-throughput analysis. MS offers a very versatile tool that can be used for drug discovery. Mass spectroscopy may be employed either alone or in combination with other means for detection or identifying the organic compound ligand bound to the target. Techniques employing mass spectroscopy are well known in the art and have been employed for a variety of applications (see, e.g., Fitzgerald and Siuzdak, Chemistry & Biology 3: 707-715 [1996]; Chu et al., J. Am. Chem. Soc. 118: 7827-7835 [1996]; Siudzak, Proc. Natl. Acad. Sci. USA 91: 11290-11297 [1994]; Burlingame et al., Anal. Chem. 68: 599R-651R [1996]; Wu et al., Chemistry & Biology 4: 653-657 [1997]; and Loo et al., Am. Reports Med. Chem. 31: 319-325 [1996]).

The Catch and Weigh Strategy

The tethering strategy elaborated by Erlanson et al. (PNAS 97:17 pp 9367-9372, August 2000) allows for the discovery of low molecular weight molecules that bind weakly to Target biological Molecules, especially proteins. This capability is mediated by a disulfide tether between the small molecule and the target protein that enhances their interaction and can be tuned by adjusting the red-ox potential. The degree of binding is quantified, and the molecule's identity determined by mass spectrum analysis of the entire complex, namely the TBM-Ligand complex, is referred to as the “catch and weigh” strategy.

The Catch and Release Strategy

The “catch and release” strategy described here is an extension of the tethering process that allows it to be applied to TBM's, especially protein targets, that do not give good or precise mass spectrum. Such cases include glycosylated proteins or otherwise heterogeneous proteins, and high disulfide-content proteins. Catch and release is achieved by employing the disulfide tethered TBM-Ligand complex to separate the bound library members from unbound library members. Thereafter the bound molecule is released with a reducing agent from the protein and labeled with a tag that facilitates its measurement and aids its identification by mass spectrometry or other methods (e.g. fluorescence).

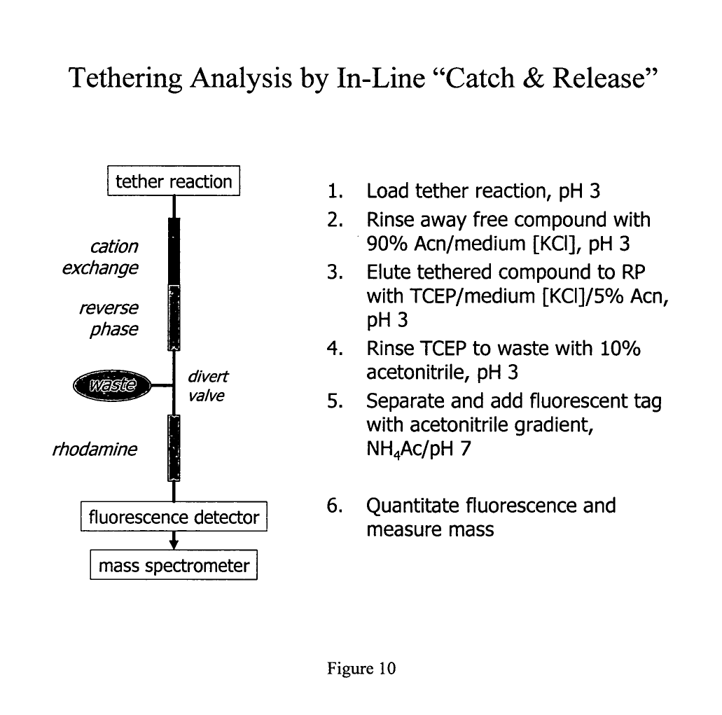

More specifically, a specific example of the catch and release strategy is diagrammed in FIG. 10. First, tether reactions that may contain a library member covalently tethered to the target protein are injected onto a cation exchange-reverse phase hybrid column under low pH conditions to capture the protein-ligand complex. Subsequently, the column is rinsed with solvent to remove untethered compounds to a waste tube. Next, the tethered compound caught by the protein is released with a reducing agent (e.g. TCEP (tris-carboxyethylphosphine), DTT, or βME) under aqueous conditions and transferred a downstream reverse phase matrix that holds the compound while the reducing agent is washed away to the waste tube. Afterwards the divert valve is closed and an acetonitrile gradient is used to transfer the compound to a rhodamine exchange column.

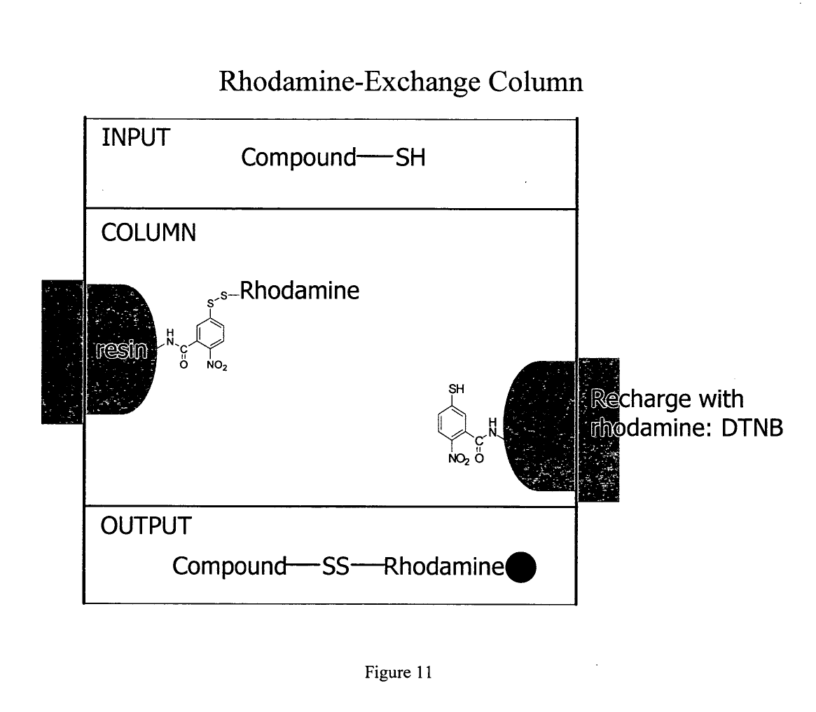

This column uses disulfide exchange with rhodamine that is attached to an immobilized leaving group to tag the released thiol compound with this fluorophore (see FIG. 11). After tagging, the compound passes though a fluorimeter to measure the amount present, and through a mass spectrophotometer to determine its identity. The positive charge imparted by the rhodamine derivatization allows the mass spectrometer to readily detect a large variety of hit compounds.

However, the scope of the invention is not limited to these particular methods for detecting ligand binding. In fact, any other suitable technique for the detection of the adduct formed between the biological target molecule and the library member can be used. For example, one may employ various chromatographic techniques, such as liquid chromatography, thin layer chromatography, and like for separation of the components of the reaction mixture so as to enhance the ability to identify the covalently bound organic molecule. Such chromatographic techniques may be employed in combination with mass spectroscopy or separate from mass spectroscopy. One may optionally couple a labeled probe (fluorescently, radioactively, or otherwise) to the liberated organic compound so as to facilitate its identification using any of the above techniques.

In another embodiment, the formation of the new bonds liberates a labeled probe, which can then be monitored. Other techniques that may find use for identifying the organic compound bound to the target molecule include, for example, nuclear magnetic resonance (NMR), capillary electrophoresis, X-ray crystallography, and the like, all of which will be well known to those skilled in the art.

Uses of Diaphores Identified

The methods described herein provide powerful techniques for generating drug leads, and allowing the identification of two or more fragments that bind weakly, or with moderate binding affinity, to a target at sites near one another, and the synthesis of diaphores or larger molecules comprising the identified fragments (monophores) covalently linked to each other to produce higher affinity compounds. The diaphores or similar multimeric compounds including further ligand compounds, are valuable tools in rational drug design, which can be further modified and optimized using medicinal chemistry approaches and structure-aided design.

The diaphores identified in accordance with the present invention and the modified drug leads and drugs designed therefrom can be used, for example, to regulate a variety of in vitro and in vivo biological processes which require or depend on the site-specific interaction of two molecules. Molecules which bind to a polynucleotide can be used, for example, to inhibit or prevent gene activation by blocking the access of a factor needed for activation to the target gene, or repress transcription by stabilizing duplex DNA or interfering with the transcriptional machinery.

### DESCRIPTION OF PREFERRED EMBODIMENTS

In a preferred embodiment, the methods described herein are used to identify low molecular weight ligands that bind to at least two different sites of interest on target proteins through intermediary disulfide tethers formed between a first ligand and the protein, and a reactive group on the first ligand and a second ligand, respectively.

The low molecular weight ligands screened in this manner will be, for the most part, small chemical molecules that will be less than about 2000 daltons in size, usually less than about 1500 daltons in size, more usually less than about 750 daltons in size, preferably less than about 500 daltons in size, often less than about 250 daltons in size, although organic molecules larger than 2000 daltons in size will also find use herein.

Organic molecules may be obtained from a commercial or non-commercial source. For example, a large number of small organic chemical compounds are readily obtainable from commercial suppliers, such as Aldrich Chemical Co., Milwaukee, Wis. and Sigma Chemical Co., St. Louis, Mo., or may be obtained by chemical synthesis. Libraries of small organic compounds carrying appropriate reactive groups are preferably screened. In one embodiment, the reactive groups are thiol or protected thiol groups.

In recent years, combinatorial libraries, typically having from dozens to hundreds of thousands of members, have become a major tool for ligand discovery and drug development. In general, libraries of organic compounds which find use herein will comprise at least 2 organic compounds, often at least about 25 different organic compounds, more often at least about 100 different organic compounds, usually at least about 300 different organic compounds, preferably at least about 2500 different organic compounds, and most preferably at least about 5000 or more different organic compounds. Populations may be selected or constructed such that each individual molecule of the population may be spatially separated from the other molecules of the population (e.g. in separate microtiter well) or two or more members of the population may be combined if methods for deconvolution are readily available. Usually, each member of the organic molecule library will be of the same chemical class (i.e. all library members are aldehydes, all library members are primary amines, etc.), however, libraries of organic compounds may also contain molecules from two or more different chemical classes.

In a preferred embodiment, the target biological molecule (TBM) is a polypeptide that contains or has been modified to contain a free thiol. Preferrably from 2-15 different single cysteine mutants of a particular TBM are produced and each mutant is screened against pools (e.g. 10 or more) of compounds of Formula I-XIV each member of the pool differing in mass by about 10 daltons from the other members of the pool. The pools are screened under redox control using mercaptoethanol as a reducing agent. Preferrably the screening conditions are as follows TBM protein concentration 2-20 μM (more preferrably 5-10 μM), total thiol concentration for pools of library compounds 200 μM-20 mM (preferrably about 1 mM), mercaptanol from 200 μM-20 mM (preferrably about 1-3 mM), at pH 7-8 with an incubation time of 2+ hours at room temperature. Any bound ligand is then identified by the “catch and weigh” or “catch and release” mass spec. method.

In another preferred embodiment, the target biological molecule (TBM) is a polypeptide that has been modified by covalently linking with a first ligand having intrinsic affinity for the TBM and containing a thiol-containing extender tether. A complex formed between the target molecule and the thiol-containing extender is then used to screen a library of disulfide-containing monophores to identify a library member that has the highest intrinsic affinity for a second binding site on the target molecule. In one embodiment, the reactive group on the modified TBM is a free thiol group contributed by the extender, and the library is made up of small molecular weight compounds containing reactive thiol group. For disulfide tethering to capture the most stable ligand, the reaction should be under rapid exchange to allow for equilibration. In a preferred embodiment, the reaction is carried out in the presence of catalytic amount of a reducing agent such as 2-mercaptoethanol. Thermodynamic equilibrium reached in the presence of a reducing agent will favor the formation of disulfide bond between thiol group of the extender on the modified TBM and thiol group of a member of the library having intrinsic affinity for the TBM. Thus, two different ligands with intrinsic affinity for two different sites on the same TBM will be covalently linked to form a diaphore. The diaphore will bind to the TBM with a higher affinity than any of the constituent monophore units. The monophore units in a diaphore may be from the same or different chemical classes. By “same chemical class” is meant that each monophore component is of the same chemical type, i.e., both are aldehyde or amines etc.

### EXAMPLES

The invention is further illustrated by the following, non-limiting examples. Unless otherwise noted, all the standard molecular biology procedures are performed according to protocols described in (Molecular Cloning: A Laboratory Manual, vols. 1-3, edited by Sambrook, J., Fritsch, E. F., and Maniatis, T., Cold Spring Harbor Laboratory Press, 1989; Current Protocols in Molecular Biology, vols. 1-2, edited by Ausbubel, F., Brent, R., Kingston, R., Moore, D., Seidman, J. G., Smith, J., and Struhl, K., Wiley Interscience, 1987).

One basic tethering approach has been described by Erlanson et al., supra, and in PCT Publication No. WO 00/00823. The “extended tethering” approach is illustrated herein using caspase-3 as a target biological molecule (TBM). Caspases are a family of cysteine proteases, that are known to participate in the initiation and execution of programmed cell death (apoptosis). The first caspase (now referred to as Caspase-1) was originally designated as interleukin-1β-converting enzyme (ICE) (Thornburry et al., Nature 356:768-774 [1992]; Cerretti et al., Science 356:97-100 [1992]). Subsequently a large number of caspases have been identified and characterized forming a caspase family. Presently there are at least 10 members in the family (Caspase-1 to Caspase-10).

Caspases are expressed in cells in an enzymatically inactive form and become activated by proteolytic cleavage in response to an apoptotic stimulus. The inactive proenzyme form consists of a large and a small domain (subunit), in addition to an inhibitory N-terminal domain. Caspase activation involves the processing of the proenzyme into the large and small subunits, which occurs internally within the molecule. Caspases are activated either by self-aggregation and autoprocessing (as in the initiation of apoptosis), or via cleavage by an activated upstream caspase (as in the execution phase of apoptosis). For review, see, for example, Cohen G. M. Biochem. J. 326: 1-16 (1997).

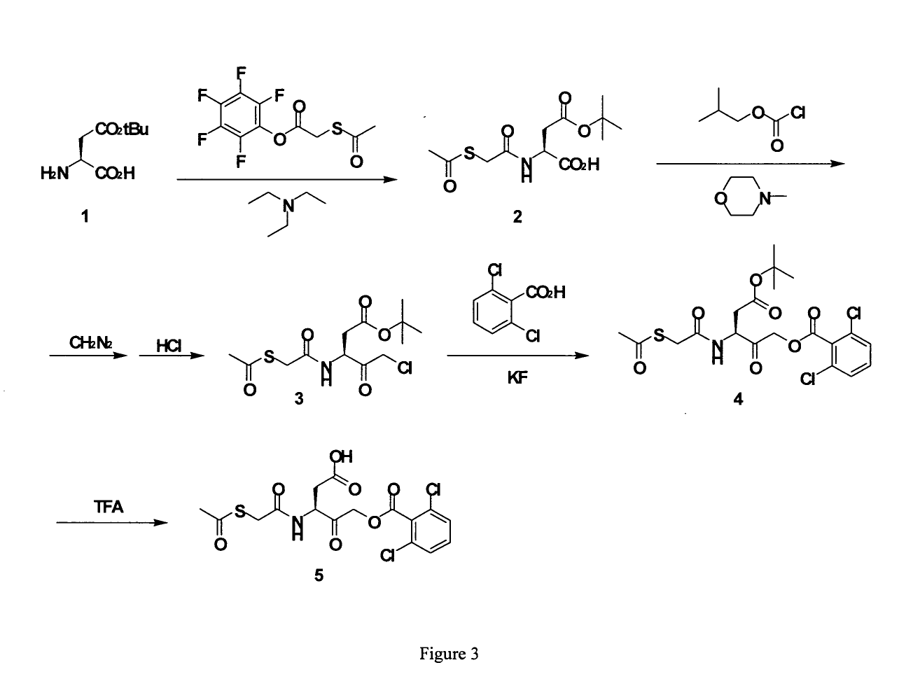

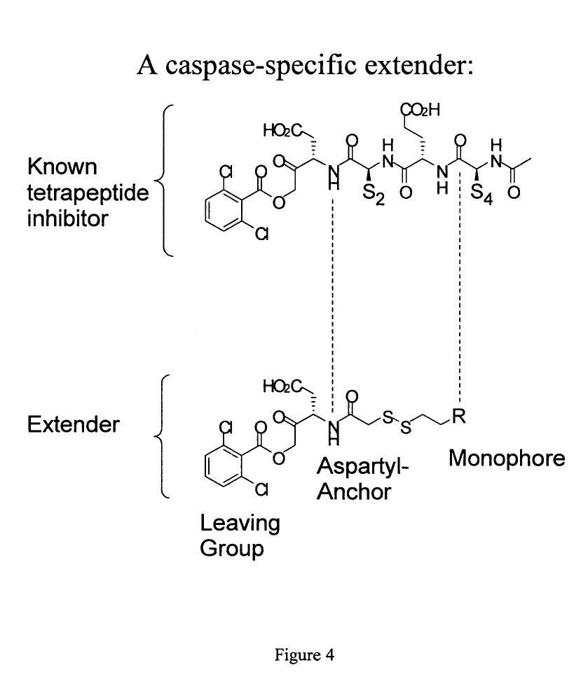

Based on a known tetrapeptide inhibitor of caspase (Ator and Dolle, Current Pharmaceutical Design, 1 191-210 (1995)) an extender was synthesized: 2,6-Dichloro-benzoic acid 3-(2-acetylsulfanyl-acetylamino)-4-carboxy-2-oxo-butyl ester (shown as compound 5 in FIG. 3), the synthesis of which is described in Example 2 below. A generic structure of extender is shown in FIG. 4. Caspase-3 was modified by reacting with the extender (Example 3) and subsequently used as a biological target molecule for screening of disulfide library prepared as described in Example 1, by using the extended tethering approach.

All commercially available materials were used as received. All synthesized compounds were characterized by ¹H NMR on a Bruker (Billerica, Mass.) DMX400 MHz Spectrometer and HPLC-MS (Hewlett-Packard Series 1100 MSD).

### Example 1

### Disulfide Libraries

Disulfide libraries were synthesized using standard chemistry from the following classes of compounds: aldehydes, ketones, carboxylic acids, amines, sulfonyl chlorides, isocyanates, and isothiocyanates. For example, the disulfide-containing library members were made from commercially available carboxylic acids and mono-N-(tert-butoxycarbonyl)-protected cystamine (mono-BOC-cystamine) by adapting the method of Parlow and coworkers (Parlow and Normansell, Mol. Diversity. 1: 266-269 [1995]). Briefly, 260 μmol of each carboxylic acid was immobilized onto 130 μmol equivalents of 4-hydroxy-3-nitrobenzophenone on polystyrene resin using 1,3-diisopropylcarbodiimide (DIC) in N,N-dimethylformamide (DMF).

After 4 hours at room temperature, the resin was rinsed with DMF (×2), dichloromethane (DCM, ×3), and tetrahydrofuran (THF, ×1) to remove uncoupled acid and DIC. The acids were cleaved from the resin via amide formation with 66 μmol of mono-BOC protected cystamine in THF. After reaction for 12 hours at ambient temperature, the solvent was evaporated, and the BOC group was removed from the uncoupled half of each disulfide by using 80% trifluoroacetic acid (TFA) in DCM. The products were characterized by HPLC-MS, and those products that were substantially pure were used without further purification.

Libraries were also constructed from mono-BOC-protected cystamine and a variety of sulfonyl chlorides, isocyanates, and isothiocyanates. In the case of the sulfonyl chlorides, 10 μmol of each sulfonyl chloride was coupled with 10.5 μmol of mono-BOC-protected cystamine in THF (with 2% diisopropyl ethyl amine) in the presence of 15 mg of poly(4-vinyl pyridine). After 48 hours, the poly(4-vinylpyridine) was removed via filtration, and the solvent was evaporated. The BOC group was removed by using 50% TFA in DCM. In the case of the isothiocyanates, 10 μmol of each isocyanate or isothiocyanate was coupled with 10.5 μmol of mono-BOC-protected cystamine in THF. After reaction for 12 hours at ambient temperature, the solvent was evaporated, and the BOC group was removed by using 50% TFA in DCM. A total of 212 compounds were made by using this methodology.

Finally, oxime-based libraries were constructed by reacting 10 μmol of specific aldehydes or ketones with 10.5 μmol of HO(CH₂)₂S—S(CH₂)₂ONH₂ in 1:1 methanol/chloroform (with 2% acetic acid added) for 12 hours at ambient temperature to yield the oxime product. A total of 448 compounds were made by using this methodology.

Individual library members were redissolved in either acetonitrile or dimethyl sulfoxide to a final concentration of 50 or 100 mM. Aliquots of each of these were then pooled into groups of 8-15 discrete compounds, with each member of the pool having a unique molecular weight.

### Example 2

### Creating an Amine Linker

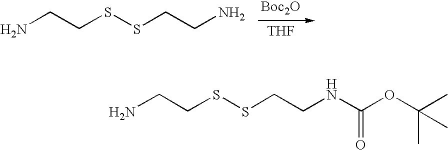

To cystamine dihydrochloride (10 g, 444 mmol) was added 5 N NaOH (400 mL) and the suspension stirred until a clear solution formed. The solution was extracted with DCM (6×200 mL) and the combined DCM layers dried (Na₂SO₄), filtered and concentrated to afford 64.5 g of the desired free base (95%).

To a solution of the free base (422 mmol) in THF (285 mL) was added dropwise a solution of di-t-butyldicarbonate (0.5 eq, 212 mmol) in THF (212 mL). The reaction was allowed to stir overnight, then concentrated to an oil, taken up in 1 M NaHSO₄ (500 mL), and washed with ethylacetate. The aqueous layer was cooled in an ice-bath, treated with 5 M NaOH (200 mL), and the resulting solution immediate washed with DCM. The DCM layers were combined, dried (Na₂SO₄), filtered and concentrated to afford 11.4 g of the desired mono-Boc cystamine (21%).

### Example 3

### Creating a Carboxylate Linker

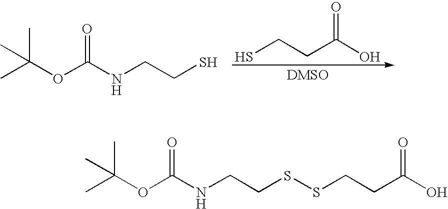

To tert-butyl N-(2-mercaptoethyl)carbamate (10 g, 56 mmol) in DMSO (20 mL) was added 3-mercaptopropionic acid (6 g, 57 mmol) and the solution heated at 70 C for 48 hours. The solution was cooled, and the resulting waxy solid dissolved in chloroform (200 mL) and washed with 5% aqueous NaHCO3 (4×50 mL). The aqueous layers were combined, carefully acidified to litmus with 1 N HCl, and washed with CHCl3 (4×50 mL). The organic layers were combined, washed with brine, dried (Na2SO4), concentrated and then purified on silica gel (9/1 DCM/MeOH) to afford 1.8 g of a colorless oil (12%).

### Example 4

### Extender Synthesis

For the extended tethering approach, extender (2,6-Dichloro-benzoic acid 3-(2-acetylsulfanyl-acetylamino)-4-carboxy-2-oxo-butyl ester, shown as compound 5 in FIG. 3) was synthesized using a series of chemical reactions as shown in FIG. 3, and described below.

### Synthesis of 2-(2-Acetylsulfanyl-acetylamino)-succinic acid 4-tert-butyl ester (compound 2, FIG. 3)

Acetylsulfanyl-acetic acid pentafluorophenyl ester (1.6 g, 5.3 mmol) and H-Asp(OtBu)-OH (1 g, 5.3 mmol) were mixed in 20 ml of dry dichloromethane (DCM). Then 1.6 ml of triethylamine (11.5 mmol) was added, and the reaction was allowed to proceed at ambient temperature for 3.5 hours. The organic layer was then extracted with 3×15 ml of 1 M sodium carbonate, the combined aqueous fractions were acidified with 100 ml of 1 M sodium hydrogensulfate and extracted with 3×30 ml ethyl acetate. The combined organic fractions were then rinsed with 30 ml of 1 M sodium hydrogensulfate, 30 ml of 5 M NaCl, dried over sodium sulfate, filtered, and evaporated under reduced pressure to yield 1.97 g of a nearly colorless syrup which was used without further purification. MW=305 (found 306, M+1).

### Synthesis of 3-(2-Acetylsulfanyl-acetylamino)-5-chloro-4-oxo-pentanoic acid tert-butyl ester (compound 3, FIG. 3)

The free acid (compound 2) was dissolved in 10 ml of dry tetrahydrofuran (THF), cooled to 0° C., and treated with 0.58 ml N-methyl-morpholine (5.3 mmol) and 0.69 isobutylchloroformate. Dense white precipitate immediately formed, and after 30 minutes the reaction was filtered through a glass frit and transferred to a new flask with an additional 10 ml of THF. Meanwhile, diazomethane was prepared by reacting 1-methyl-3-nitro-1-nitrosoguanidine (2.3 g, 15.6 mmol) with 7.4 ml of 40% aqueous KOH and 25 ml diethyl ether for 45 minutes at 0° C. The yellow ether layer was then decanted into the reaction containing the mixed anhydride, and the reaction allowed to proceed while slowly warming to ambient temperature over a period of 165 minutes.

The reaction was cooled to 8° C., and 1.5 ml of 4 N HCl in dioxane (6 mmol total) was added dropwise. This resulted in much bubbling, and the yellow solution became colorless. The reaction was allowed to proceed for two hours while gradually warming to ambient temperature and then quenched with 1 ml of glacial acetic acid. The solvent was removed under reduced pressure and the residue redissolved in 75 ml ethyl acetate, rinsed with 2×50 ml saturated sodium bicarbonate, 50 ml 5 M NaCl, dried over sodium sulfate, filtered, and evaporated to dryness before purification by flash chromatography using 90:10 chloroform:ethyl acetate to yield 0.747 g of light yellow oil (2.2 mmol, 42% from (1)). Expected MW=337.7, found 338 (M+1).

### Synthesis of 2,6-Dichloro-benzoic acid 3-(2-acetylsulfanyl-acetylamino)-4-tert-butoxycarbonyl-2-oxo-butyl ester (compound 4, FIG. 3)

The chloromethylketone (compound 3) (0.25 g, 0.74 mmol) was dissolved in 5 ml of dry N,N-dimethylformamide (DMF), to which was added 0.17 g 2,6-dichlorobenzoic acid (0.89 mmol) and 0.107 g KF (1.84 mmol). The reaction was allowed to proceed at ambient temperature for 19 hours, at which point it was diluted with 75 ml ethyl acetate, rinsed with 2×50 ml saturated sodium bicarbonated, 50 ml 1 M sodium hydrogen sulfate, 50 ml 5 M NaCl, dried over sodium sulfate, filtered, and dried under reduced pressure to yield a yellow syrup which HPLC-MS revealed to be about 75% product and 25% unreacted (3). This was used without further purification. Expected MW=492.37, found 493 (M+1).

### Synthesis of 2,6-Dichloro-benzoic acid 3-(2-acetylsulfanyl-acetylamino)-4-carboxy-2-oxo-butyl ester (compound 5, FIG. 3)

The product of the previous step (compound 4) was dissolved in 10 ml of dry DCM, cooled to 0° C., and treated with 9 ml trifluoroacetic acid (TFA). The reaction was then removed from the ice bath and allowed to warm to ambient temperature over a period of one hour. Solvent was removed under reduced pressure, and the residue redissolved twice in DCM and evaporated to remove residual TFA. The crude product was purified by reverse-phase high-pressure liquid chromatography to yield 101.9 mg (0.234 mmol, 32% from (3)) of white hygroscopic powder. Expected MW=436.37, found 437 (M+1). This was dissolved in dimethylsulfoxide (DMSO) to yield a 50 mM stock solution.

### Example 5

### Modification of Caspase 3 with Extender

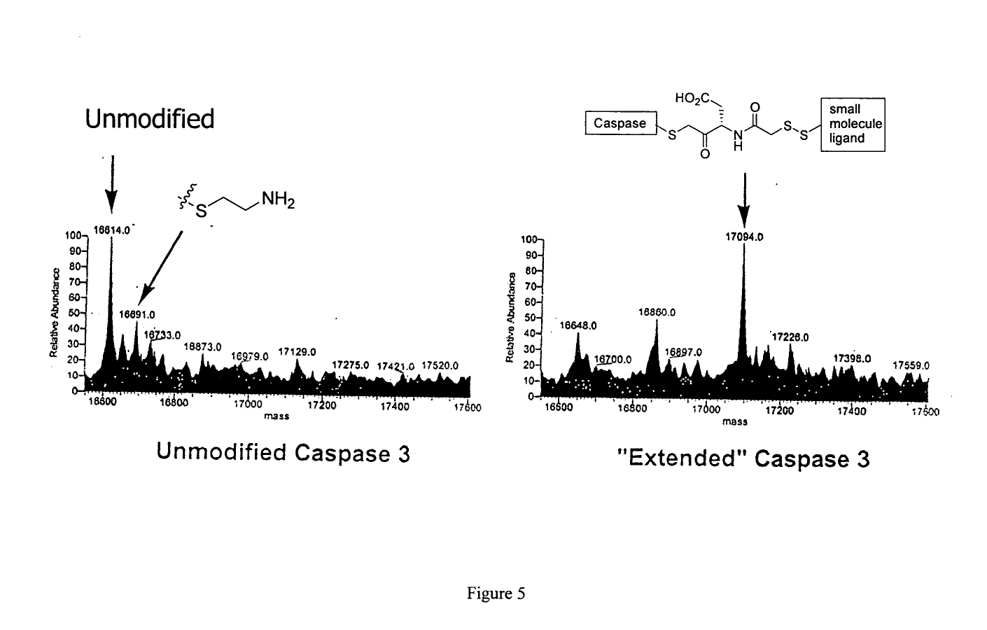

Caspase 3 was cloned, overexpressed, and purified using standard techniques (Rotonda et al. Nature Structural Biology 3(7):619-625 (1996)). To 2 ml of a 0.2 mg/ml Caspase 3 solution was added 10 ml of 50 mM 2,6-Dichloro-benzoic acid 3-(2-acetylsulfanyl-acetylamino)-4-carboxy-2-oxo-butyl ester (compound 5, FIG. 3) synthesized as described in Example 2, and the reaction was allowed to proceed at ambient temperature for 3.5 hours, at which point mass spectroscopy revealed complete modification of the caspase 3 large subunit (MW 16861Da, calculated MW 16860Da). The thioester was deprotected by adding 0.2 ml of 0.5 M hydroxylamine buffered in PBS buffer, and allowing the reaction to proceed for 18 hours, at which point the large subunit had a mass of 16819Da (calculated 16818Da). The protein was concentrated in a Ultrafree 5 MWCO unit (Millipore) and the buffer exchanged to 0.1 M TES pH 7.5 using a Nap-5 column (Amersham Pharmacia Biotech). The structure of the resulting “extended” caspase-3 is shown in FIG. 5.

The protein was then screened against a disulfide library prepared as described above, in Example 1, and using the methodology described in Example 6 below.

### Example 6

### Screening of Disulfide Library

In a typical experiment, 1 μl of a DMSO solution containing a library of 8-15 disulfide-containing compounds was added to 49 μl of buffer containing extender-modified protein. When mass spectroscopy was used for the identification of the bound ligand, the compounds were chosen so that each has a unique molecular weight. For example, these molecular weights differ by at least 10 atomic mass units so that deconvolution is unambiguous. Although pools of 8-15 disulfide-containing compounds were typically chosen for screening because of the ease of deconvolution, larger pools can also be used. The protein was present at a concentration of 15 μM, each of the disulfide library members was present at ˜0.2 mM, and thus the total concentration of all disulfide library members was ˜2 mM. The reaction was done in a buffer containing 25 mM potassium phosphate (pH 7.5) and 1 mM 2-mercaptoethanol, although other buffers and reducing agents can be used.

The reactions were allowed to equilibrate at ambient temperature for at least 30 min. These conditions can be varied considerably depending on the ease with which the protein ionizes in the mass spectrometer, the reactivity of the specific cysteine(s), etc.

After equilibration of aspartyl-conjugated caspase-3 (Example 3) and library (Example 1), the reaction was injected onto an HP1100 HPLC and chromatographed on a C₁₈ column attached to a mass spectrometer (Finnigan-MAT LCQ, San Jose, Calif.). The multiply charged ions arising from the protein were deconvoluted with available software (XCALIBUR) to arrive at the mass of the protein. The identity of any library member bonded through a disulfide bond to the protein was then easily determined by subtracting the known mass of the unmodified protein from the observed mass. This process assumes that the attachment of a library member does not dramatically change the ionization characteristics of the protein itself, a conservative assumption because in most cases the protein will be at least 20-fold larger than any given library member. This assumption was confirmed by demonstrating that small molecules selected by one protein are not selected by other proteins.

The results of a representative experiment are shown in FIG. 5. The spectrum on the right side of FIG. 5 shows the result of reacting “extended” caspase-3 (synthesized as described in Example 3, with a disulfide-containing molecule identified from a pool as modifying extended Caspase-3. The predominant peak obtained (mass of 17,094 Da) corresponds to caspase-3 covalently linked to the small molecule ligand, which has an intrinsic affinity for a second site of interest on caspase-3, resulting in the diaphore compound shown above the peak.

The mass spectrum shown on the left side is a deconvoluted mass spectrum of unmodified caspase-3 (a cysteine-containing polypeptide target) the same disulfide-containing small molecule ligand used above. The spectrum reveals a predominant peak corresponding to the mass of unmodified caspase-3 (16,614 Da). A significantly smaller peak represents caspase-3 disulfide-bonded to 2-aminoethanethiol (combined mass: 16691 Da). Note that here the small molecule ligand is not selected because its binding site is too far from the reactive cysteine and no extender has been introduced. The initial lead compound, identified as described above, was then modified in order to evaluate the relative importance of various substituents in specific binding to caspase-3.

### Example 7

### Cloning of Human Caspase-3

The human version of caspase-3 (also known as Yama, CPP32 beta) was cloned directly from Jurkat cells (Clone E6-1; ATCC). Briefly, total RNA was purified from Jurkat cells growing at 37° C./5% CO₂ using Tri-Reagent (Sigma). Oligonucleotide primers were designed to allow DNA encoding the large and small subunits of Caspase-3/Yama/CPP32 to be amplified by polymerase chain reaction (PCR). Briefly, DNA encoding amino acids 28-175 (encompassing most of the large subunit) was directly amplified from 1 μg total RNA using Ready-To-Go-PCR Beads (Amersham/Pharmacia) and the following oligonucleotides:

(SEQ ID NO: 1)

5′-TTCCATATGTCTGGAATATCCCTGGACAACAGTTA-3′
and

(SEQ ID NO: 2)

5′-AAGGAATTCTTAGTCTGTCTCAATGCCACAGTCCAG-3′.

DNA encoding amino acids 176-277 (encompassing most of the small subunit) was directly amplified from 1 μg total RNA using Ready-To-Go-PCR Beads (Amersham/Pharmacia) and the following oligonucleotides:

(SEQ ID NO: 3)

5′-TTCCATATGAGTGGTGTTGATGATGACATGGCG-3′
and

(SEQ ID NO: 4)

5′-AAGGAATTCTTAGTGATAAAAATAGAGTTCTTTTGTGAG-3′

Amplified DNA corresponding to either the large subunit or the small subunit of caspase-3 was then cleaved with the restriction enzymes EcoRI and NdeI and directly cloned using standard molecular biology techniques into pRSET-b (Invitrogen) digested with EcoRI and NdeI. [See e.g. Tewari M, Quan L T, O'Rourke K, Desnoyers S, Zeng Z, Beidler D R, Poirier G G, Salvesen G S and Dixit V M. Yama/CPP32 beta, a mammalian homolog of CED-3, is a CrmA-inhibitable protease that cleaves the death substrate poly (ADP-ribose) polymerase Cell 81 (5), 801-809 (1995)]

Preparation of Single Stranded DNA

Plasmids containing DNA encoding either the large or small subunits of Caspase-3 were separately transformed into E. coli K12 CJ236 cells (New England BioLabs) and cells containing each construct were selected by their ability to grow on ampicillin containing agar plates. Overnight cultures of the large and small subunits were individually grown in 2YT (containing 100 μg/mL of ampicillin) at 37° C. Each culture was diluted 1:100 and grown to A₆₀₀=0.3-0.6. A 1.5 mL sample of each culture was removed and infected with 10 μL of phage VCS-M13 (Stratagene), shaken at 37° C. for 60 minutes, and an overnight culture of each was prepared with 1 mL of the infected culture diluted 1:100 in 2YT with 100 μg/mL of ampicillin and 20 μg/ml of chloramphenicol and grown at 37° C. Cells were centrifuged at 3000 rcf for 10 minutes and ⅕ volume of 20% PEG/2.5M NaCl was added to the supernatant. Samples were incubated at room temperature for 10 minutes and then centrifuged at 4000 rcf for 15 minutes. The phage pellet was resuspended in PBS and spun at 15 K rpm for 10 minutes to remove remaining particulate matter. Supernatant was retained, and single stranded DNA was purified from the supernatant following procedures for the QIA prep spin M13 kit (Qiagen).

Identification of Residues to be Modified to Cysteine Residues

Selection of amino acid residues that were modified to cysteine residues was made by examining the three-dimensional crystal structure of caspase-3. Residues were chosen based on the criteria that compounds identified as hits in the tethering screen would likely interact with residues within the catalytic active site. Nine different amino acid residues were chosen for modification to cysteine residues. Each version of caspase-3 harboring cysteine mutations was expressed at high levels in E. coli cells (generally >1 mg/l). In all but one case we were able to successfully purify correctly refolded tetrameric protein (as assessed by its ability to be purified by Uno-5 Q chromatography). However, caspase-3 protein containing a histidine to cysteine mutation at amino acid 121 of the large subunit could not be purified by conventional chromatography. Since we were able to purify each subunit individually, we reasoned that this was most likely due to the inability of this variant form of caspase-3 to correctly form a tetramer (i.e. to refold the large with the small subunit). We also found that not all versions of caspase-3 bearing novel cysteine residues were catalytically active, for instance, Y204C is catalytically inactive.

Single Stranded Mutagenesis

Cysteine mutations in the small subunit were made with the corresponding primers:

| | |
|---|---|
| F256C | |
| (5′-CTT TGC ATG ACA AGT AGC GTC-3′), | (SEQ ID NO: 5) |
| S209C | |
| (5′-GCC ATC CTT ACA ATT TCG CCA-3′), | (SEQ ID NO: 6) |
| S251C | |
| (5′-AGC GTC AAA GCA AAA GGA CTC-3′), | (SEQ ID NO: 7) |
| W214C | |
| (5′-CTG GAT GAA ACA GGA GCC ATC-3′), | (SEQ ID NO: 8) |
| and | |
| Y204C | |
| (5′-TCG CCA AGA ACA ATA ACC AGG-3′). | (SEQ ID NO: 9) |

Cysteine mutations in the large subunit were made with the corresponding primers:

H121C

(SEQ ID NO: 10)

(5′-TTC TTC ACC ACA GCT CAG AAG-3′),
L168C

(SEQ ID NO: 11)

(5′-GCC ACA GTC ACA TTC TGT ACC-3′),
M61C

(SEQ ID NO: 12)

(5′-CCG AGA TGT ACA TCC AGT GCT-3′),
and
S65C

(SEQ ID NO: 13)

(5′-ATC TGT ACC ACA CCG AGA TGT-3′).

Approximately 100 pmol of each primer was phosphorylated by incubating at 37° C. for 60 minutes in buffer containing 1×TM Buffer (0.5M Tris pH 7.5, 0.1M MgCl₂), 1 mM ATP, 5 mM DTT, and 5 U T4 Kinase (NEB). Kinased primers were annealed to the template DNA in a 20 μL reaction volume (˜50 ng kinased primer, 1×TM Buffer, and 10-50 ng single-stranded DNA) by incubation at 85° C. for 2 minutes, 50° C. for 5 minutes, and then at 4° C. for 30-60 minutes. An extension cocktail (2 mM ATP, 5 mM dNTP's, 30 mM DTT, T4 DNA Ligase (NEB), and T7 Polymerase (NEB)) was added to each annealing reaction and incubated at room temperature for 3 hours. Mutagenized DNA was transformed into E. coli XL1-Blue cells, and colonies containing plasmid DNA selected were for by growth on LB agar plates containing 100 μg/ml ampicillin. DNA sequencing was used to identify plasmids containing the appropriate mutation.

Protein Expression and Purification

Plasmid DNA encoding cysteine mutations in the large subunit were transformed into Codon Plus BL21 Cells and plasmid DNA encoding cysteine mutations in the small subunit were transformed into BL21 (DE3) pLysS Cells. Codon Plus BL21 Cells containing plasmids encoding wild-type and cysteine mutated versions of the large subunit were grown in 2YT containing 150 μg/mL of ampicillin overnight at 37° C. and immediately harvested. BL21 pLysS Cells containing plasmids encoding wild-type and cysteine mutated versions of the small subunit were grown in 2YT at 37° C. with 150 μg/mL of ampicillin until A₆₀₀=0.6. Cultures were subsequently induced with 1 mM IPTG and grown for an additional 3-4 hours at 37° C. After harvesting cells by centrifuging at 4K rpm for 10 minutes, the cell pellet was resuspended in Tris-HCl (pH8.0)/5 mM EDTA and micro fluidized twice. Inclusion bodies were isolated by centrifugation at 9K rpm for 10 minutes and then resuspended in 6M-guanidine hydrochloride. Denatured subunits were rapidly and evenly diluted to 100 μg/mL in renaturation buffer (100 mM Tris/KOH (pH 8.0), 10% sucrose, 0.1% CHAPS, 0.15M NaCl, and 10 mM DTT) and allowed to renature by incubation at room temperature for 60 minutes with slow stirring.

Renatured proteins were dialyzed overnight in buffer containing 10 mM Tris (pH8.5), 10 mM DTT, and 0.1 mM EDTA. Precipitate was removed by centrifuging at 9K rpm for 15 minutes and filtering the supernatant through a 0.22 μm Cellulose Nitrate filter. The supernatant was then loaded onto an anion-exchange column (Uno5 Q-Column (BioRad)), and correctly folded caspase-3 protein was eluted with a 0-0.25 M NaCl gradient at 3 mL/minute. Aliquots of each fraction were electrophoresised on a denaturing polyacrylamide gel and fractions containing Caspase-3 protein were pooled.

### Example 8

### Human Interleukin-2

Cloning of IL-2

Numbering of the wild type and variant IL-2 residues follows the convention of the first amino acid residue (A) of the mature protein being residue number 1 independent of any presequence e.g. met for the E. coli produced protein. (See Taniguchi et al. Nature 302 (5906) 305-310 (1983) and Devos et al. Nucleic Acids Res. 11 (13), 4307-4323 (1983))

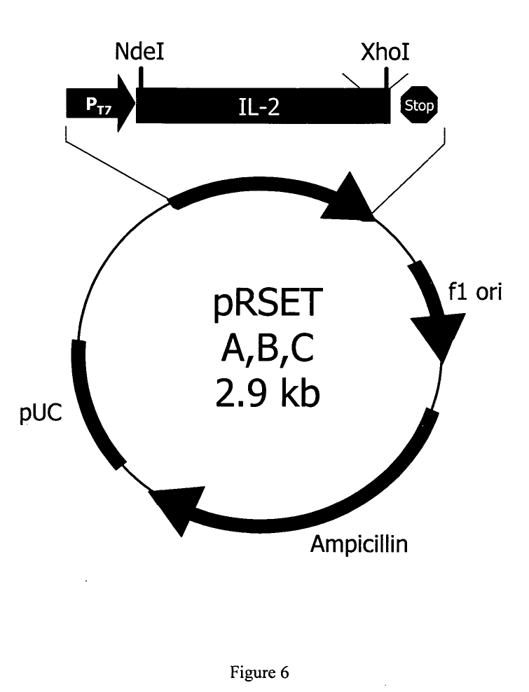

The DNA sequence encoding human Interleukin-2 (IL-2) was isolated from plasmid pTCGF-11 (ATCC Accession No. 39673). PCR primers (IL2 Forward-ggaattccatatggcacctacttcaagttcta caaagaaaaca (SEQ ID NO:14); IL2 Reverse-ccgctcgagtcaagttagtgttgagatgatgctttgaca) (SEQ ID NO:15) were designed to contain restriction endonuclease sites NdeI and XhoI for subcloning into a pRSET expression vector (Invitrogen). A map of the pRSET/IL2 vector is shown in FIG. 6. Double-stranded IL-2/pRSET was prepared as follows:

The PCR product containing the IL-2 sequence and pRSET were both cut with restriction endonucleases (1 μl PCR product, 1 μl each endonuclease, 2 μM appropriate 10× buffer, 15 μl water; incubated at 37 degrees C. for 2 hrs). The products of nuclease cleavage were isolated from an agarose gel (1% agarose, TAE buffer) and ligated together using T4 DNA ligase (80 ng IL-2 sequence, 160 ng pRSET vector, 4 μl 5× ligase buffer [300 mM Tris pH 7.5, 50 mM MgCl₂, 20% PEG 8000, 5 mM ATP, 5 mM DTT], 1 μl ligase; incubated at 15 degrees C. for 1 hour). 10 μl of the ligase reaction mixture was transformed into XL1 blue cells (Stratagene, La Jolla, Calif.) (10 μl reaction mixture, 10 115×KCM (0.5 M KCl, 0.15 M CaCl₂, 0.25 M MgCl₂), 30 μl water, 50 μl PEG-DMSO competent cells; incubated at 4 degrees C. for 20 minutes, 25 degrees C. for 10 minutes), and plated onto LB/agar plates containing 100 μg/ml ampicillin.

After incubation at 37 degrees overnight, single colonies were grown in 5 ml 2YT media for 18 hours. Cells were then isolated and double-stranded DNA extracted from the cells using a Qiagen DNA miniprep kit.

Identification of Residues to be Modified to Cysteine Residues

Selection of amino acid residues that were modified to cysteine residues was made by examining the three-dimensional crystal structure of IL-2. Residues were chosen based on the criteria that compounds identified as hits in the tethering screen would likely interact with residues within the catalytic active site.

Cloning of cys Variants of IL-2

Site-directed variants of IL-2 were prepared by the single-stranded DNA method (modification of Kunkel, PNAS, 1985). Oligonucleotides were designed to contain the desired mutations and 15-20 bases of flanking sequence.

The single-stranded form of the IL-2/pRSET plasmid was prepared by transformation of double-stranded plasmid into the CJ236 cell line (1 μl IL-2/pRSET double-stranded DNA, 2 μl 2×KCM salts, 7 μl water, 10 μl PEG-DMSO competent CJ236 cells; incubated at 4 degrees C. for 20 minutes and 25 degrees C. for 10 minutes; plated on LB/agar with 100 μg/ml ampicillin and incubated at 37 degrees C. overnight). Single colonies of CJ236 cells were then grown in 50 ml 2YT media to midlog phase; 5 μl VCS helper phage (Stratagene) were then added and the mixture incubated at 37 degrees C. overnight. Single-stranded DNA was isolated from the supernatant by precipitation of phage (⅕ volume 20% PEG 8000/2.5M NaCl; centrifuge at 12K for 15 min.). Single-stranded DNA was then isolated from phage using Qiagen single-stranded DNA kit. Sequencing (see below) identified a serine-25 to leucine mutation, which was corrected by mutagenesis (see below) using the “S25L” (oligonucleotide S25L-ttaattccattcaaaatcatctgtaf) (SEQ ID NO:16).

MUTAGENIC OLIGONUCLEOTIDES

| | | |
|---|---|---|
| N30C | ggtgagtttgggattcttgtaacaattaa | (SEQ ID NO:17) |
| | ttccattcaaaatcatctg | |
| Y31C | ggtgagtttgggattcttacaattattaa | (SEQ ID NO:18) |
| | ttccattc | |
| N33C | cctggtgagtttgggacacttgtaattat | (SEQ ID NO:19) |
| | taattcc | |
| K32C | ggtgagtttgggattacagtaattattaa | (SEQ ID NO:20) |
| | ttcc | |
| K35C | gcatcctggtgagacagggattcttgtaa | (SEQ ID NO:21) |
| | ttattaattcc | |
| R38C | cttaaatgtgagcatacaggtgagtttgg | (SEQ ID NO:22) |
| | gattc | |
| F42C | gggcatgtaaaacttacatgtgagcatcc | (SEQ ID NO:23) |
| | tgg | |
| K43C | cttgggcatgtaaaaacaaaatgtgagca | (SEQ ID NO:24) |
| | tcc | |
| Y45C | ggccttcttgggcatacaaaacttaaatg | (SEQ ID NO:25) |
| | tgagc | |
| E68C | ctcaaacctctggagtgtgtgctaaattt | (SEQ ID NO:26) |
| | agc | |
| L72C | gtttttgctttgagcacaatttagcactt | (SEQ ID NO:27) |
| | cctcc | |
| N77C | cctgggtcttaagtgaaaacatttgcttt | (SEQ ID NO:28) |
| | gagctaaatttagc | |
| Y31C | gggcatgtaaaaacaaaatgtgagcatcc | (SEQ ID NO:29) |
| K43C | tggtgagtttgggattcttacaattatta | |
| | attcc | |
| L72C | using K43C and L72C oligos | |
| K43C | | |

Site-directed mutagenesis was accomplished as follows: Oligonucleotides were dissolved to a concentration of 100D and phosphorylated on the 5′ end (2 μl oligonucleotide, 2 μl 10 mM ATP, 2 μl 10× tris-magnesium chloride buffer, 1 μl 100 mM DTT, 10 μl water, 1 μl T4 PNK; incubate at 37 degrees C. for 45 min.). Phosphorylated oligonucleotides were then annealed to single-stranded DNA template (2 μl single-stranded plasmid, 1 μl oligonucleotide, 1 μl 10×TM buffer, 6 μl water; heat at 94 degrees C. for 2 min., 50 degrees C. for 5 min., cool to room temperature). Double-stranded DNA was then prepared from the annealed oligonucleotide/template (add 2 μl 10×TM buffer, 2 μl 2.5 mM dNTPs, 1 μl 100 mM DTT, 1.5 μl 10 mM ATP, 4 μl water, 0.4 μl T7 DNA polymerase, 0.6 μl T4 DNA ligase; incubate at room temperature for two hours). E. coli (XL1 blue, Stratagene) was then transformed with the double-stranded DNA (1 μL double-stranded DNA, 10 μl 5×KCM, 40 μl water, 50 μl DMSO competent cells; incubate 20 min. at 4 degrees C., 10 min. at room temperature), plated onto LB/agar containing 100 μg/ml ampicillin, and incubated at 37 degrees C. overnight. Approximately four colonies from each plate were used to innoculate 5 ml 2YT containing 100 μg/ml ampicillin; these cultures were grown at 37 degrees C. for 18-24 hours. Plasmids were then isolated from the cultures using Qiagen miniprep kit. These plasmids were sequenced to determine which IL-2/pRSET clones contained the desired mutation.

Sequencing of IL-2 genes was accomplished as follows: The concentration of plasmid DNA was quantitated by absorbance at 280 nm. 400 ng of plasmid was mixed with sequencing reagents (1 μl DNA, 10 μl water, 1 μl sequencing primer, 8 μl sequencing mixture with Big Dye [Applied Biosystems]). The sequencing primers are shown below. The mixture was then run through a PCR cycle (96 degrees, 10 s; 50 degrees, 5 s; 60 degrees 4 min; 25 cycles) and the DNA reaction products were precipitated (20 μl mixture, 16 μl water, 64 μl ethanol; incubated 15 min at room temperature, pelleted at 12 K rpm for 20 min; wash with 250 μl 70% ethanol; heat 1 min at 94 degrees C.). The precipitated products were then suspended in TSB (Applied Biosystems) and the sequence read and analyzed by an Applied Biosystems 310 capillary gel sequencer. In general, 3-4 of the plasmids contained the desired mutation.

### SEQUENCING PRIMERS

| | |
|---|---|
| Forward primer, | |
| “T7”- AATACGACTCACTATAG | (SEQ ID NO:30) |
| Reverse primer, | |
| “RSET REV”- TAGTTATTGCTCAGCGGTGG | (SEQ ID NO:31) |

Expression of cys Variants of IL-2

Variant proteins were expressed as follows: IL-2/pRSET clones containing the mutation were transformed into BL21 DE3 pLysS cells (Invitrogen) (1 μl double-stranded DNA, 2 μl 5×KCM, 7 μl water, 10 μl DMSO competent cells; incubate 20 min. at 4 degrees C., 10 min. at room temperature), plated onto LB/agar containing 100 μg/ml ampicillin, and incubated at 37 degrees C. overnight. 10 ml cultures in 10 ml 2YT with 100 μg/ml ampicillin were grown overnight from single colonies. 100 ml 2YT/ampicillin (100 μg/ml) was innoculated with these overnight cultures and incubated at 37 degrees C. for 3 hours. This culture was then added to 1.5 L 2YT/ampicillin (100 μg/ml) and incubated until late-log phase (absorbance at 600 nm ˜0.8), at which time IPTG was added to a final concentration of 1 mM. Cultures were incubated at 37 degrees for another 3 hours and then cells were pelleted (10 Krpm, 10 min) and frozen at −20 degrees C. overnight.

IL-2 variants were then purified from the frozen cell pellets. First, cells were lysed in a microfluidizer (100 ml tris EDTA buffer, 3 passes through a Microfluidizer [Microfluidics 110S]) and inclusion bodies were isolated by precipitation (10 Krpm, 10 min.). Following cell lysis, 50 μl of cell material was saved for analysis by SDS-PAGE. All variants expressed as determined by the SDS gel but several (e.g. E68C) precipitated on refolding. Inclusion bodies were then resuspended in 45 ml guanidine HCl and spun at 10 Krpm for 10 min. The supernatant was added to refolding buffer (45 ml guanidine HCl, 36 ml tris pH 8, 231 mg cysteamine, 46 mg cystamine, 234 ml water) and incubated at room temperature for 3-5 hours. The mixture was then spun at 10 Krpm for 20 min. and the supernatant dialyzed 4-5 times in 5 volumes of buffer (10 mM ammonium acetate pH 6, 25 mM NaCl). The protein solution was then filtered through cellulose and injected onto an S Sepharose fast flow column (2.5 cm diameter×14 cm long) at 5 ml/min. The protein was then eluted using a gradient of 0-75% buffer B over 60 minutes (Buffer A: 25 mM NH₄OAc, pH 6, 25 mM NaCl; Buffer B: 25 mM NH₄OAc, pH 6, 1 M NaCl). Purified protein was then exchanged into the appropriate buffer for the TETHER assay (typically 100 mM Hepes, pH 7.4). Average yields were 0.5 to 4 mg per liter culture.

### Example 9

### The IL-1 Receptor

Cloning of Human IL-1 Receptor Type I

Numbering of the wild type and variant IL-1 receptor residues follows the convention of the first amino acid residue (A) of the mature protein being residue number 1 independent of any presequence e.g. met for the E. coli produced protein. (See Taniguchi et al. Nature 302 (5906) 305-310 (1983) and Devos et al. Nucleic Acids Res. 11 (13), 4307-4323 (1983))

The DNA sequence encoding human Interleukin-1 receptor (IL1R) was isolated by PCR from a HepG2 (ATCC Accession No. HB-8065) cDNA library using PCR primers (IL1RsigintFor 5′-ttactcagacttatttgtttcatagctcta; (SEQ ID NO: 32) IL1RintRev 5′-gaaattagtgactggatatattaactggat) (SEQ ID NO: 33) corresponding to the signal sequence (methionine at −17) and the end of the extracellular domain of the protein (lysine 329). The appropriate sized band was isolated from an agarose gel and used as the template for a second round of PCR using oligos:

IL1Rsig Forward

(SEQ ID NO: 34)

5′- ccggaattcatgaaagtgttactcagacttatttgtttc;
IL1R319 Reverse

(SEQ ID NO: 35)

5′- ccgctcgagtcacttctggaaattagtgactggatatattaa)

These oligos were designed to contain restriction endonuclease sites EcoRI and XhoI for subcloning into a pFBHT vector. The pFBHT vector is modified from the original pFastBac1 (Gibco/BRL) by cloning the sequence for TEV protease followed by (His)₆ tag and a stop signal into the XhoI and HinDIII sites. The PCR product containing the IL1R sequence was cut with restriction endonucleases (41 μl PCR product, 2 μl each endonuclease, 5 μl appropriate 10× buffer; incubated at 37° C. for 90 minutes). The pFBHT vector was cut with restriction endonucleases (6 μg DNA, 4 μl each endonuclease, 10 μl appropriate 10× buffer, water to 100 μl; incubated at 37° C. for 2 hours; add 2 μl CIP and incubated at 37° C. for 45 minutes).

The products of nuclease cleavage were isolated from an agarose gel (1% agarose, TBE buffer) and ligated together using T4 DNA ligase (200 ng pFBHT vector, 150 ng IL1R PCR product, 4 μl 5× ligase buffer [300 mM Tris pH7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4 degrees C. for 20 minutes, 25 degrees C. for 10 minutes), and plated onto LB/agar plates containing 100 μg/ml ampicillin. After incubation at 37 degrees overnight, single colonies were grown in 3 ml 2YT media for 18 hours. Cells were then isolated and double-stranded DNA extracted from the cells using a Qiagen DNA miniprep kit.

Sequencing IL-1 Receptor Wild-Type and Variants

Sequencing of IL-1R genes was accomplished as follows: The concentration of plasmid DNA was quantitated by absorbance at 280 nm. 400 ng of plasmid was mixed with sequencing reagents (1 μl DNA, 10 μl water, 1 μl sequencing primer, 8 μl sequencing mixture with Big Dye [Applied Biosystems]). The sequencing primers are shown in Table 1. The mixture was then run through a PCR cycle (96 degrees, 10 s; 50 degrees, 5 s; 60 degrees 4 min; 25 cycles) and the DNA reaction products were precipitated (20 μl mixture, 16 μl water, 64 μl ethanol; incubated 15 min at room temperature, pelleted at 12 K rpm for 20 min; wash with 250 μl 70% ethanol; heat 1 min at 94 degrees C.). The precipitated products were then suspended in TSB (Applied Biosystems) and the sequence read and analyzed by an Applied Biosystems 310 capillary gel sequencer.

TABLE 1
Sequencing Primers

| | Primer Sequence | |
|---|---|---|
| Primers | (5′ TO 3′) | |
| T7 Forward | AATACGACTCACTATAG | (SEQ ID NO: 36) |
| RSET REV | TAGTTATTGCTCAGCGGTGG | (SEQ ID NO: 37) |
| FB Forward | TATTCCGGATTATTCATACC | (SEQ ID NO: 38) |
| FB Reverse | CCTCTACAAATGTGGTATGGC | (SEQ ID NO: 39) |

Site of Interest:

The site of interest for the type-I IL-1 receptor (IL-1RI) was defined from crystal structures of the receptor bound to IL-1β (PDB code 1ITB) (Vigers, G. P. A. et al. Nature 386, 190-194 (1997)), the receptor antagonist protein (IL-1ra; PDB code 1IRA) (Schreuder, H. et al. Nature 386, 194-200 (1997)), and a peptide inhibitor discovered by phage display (PDB code 1G0Y), (Vigers, G. P. A. et al. J. Biol. Chem. 275, 36927-36933 (2000)) coupled with mutational studies of IL-1β and IL-1Ra (Evans, R. J. et al. J. Biol. Chem. 270, 11477-11483 (1995)). Critical contact residues for IL-1β include Q15, H30, Q32, and K93; critical contact residues for IL-1Ra include Q20, Y34, and Q36. Residues Q15, H30, Q32 of IL-1β make the same receptor contacts as residues Q20, Y34, and Q36 of IL-1Ra. Residue K93 of IL-1β contacts a region in the third domain of IL-1RI that is not occupied by IL-1Ra. A co-crystallized structure of IL-1RI bound to a peptide inhibitor (AF10847; ETPFTWEESNAYYWQPYALPL) (SEQ ID NO: 40) shows that residues Y13 and Q15 of the peptide occupy the same positions as H30 and Q32 of IL-1β, and Y34 and Q36 of IL-1Ra, respectively. The site occupied by Q15 of IL-1β and Q20 of IL-1Ra is missing in the peptide complex structure due to a backbone rearrangement of IL-1RI residues 112-122.

In addition, residue W6 of the peptide occupies a hydrophobic pocket on the receptor that is not occupied by any portion of IL-1β or IL-1Ra. Because the contacts made by the peptide are deemed most relevant for small-molecule design, the site of interest for IL-1RI is defined as the union of all surface-exposed residues on domains 1 and 2 of the receptor (residues 6-201) that have a CB atom within 10 Ångstroms of W6, Y13, and Q15 of the peptide, considering either the peptide-bound (1G0Y) or the IL-1β-bound (1ITB) structures. A “surface-exposed” residue is defined one that has a side chain solvent accessibility of at least 15% according to the method of Lee and Richards. According to this definition, potential 10-Ångstrom variants for IL-1RI include E11C, I13C, I14C, V16C, S18C, P26C, P28C, K63C, K95C, Y105C, N106C, A107C, Q108C A109C I110C, F111C, K112C, Q113C, K114C, V124C, P126C, Y127C, E129C, F130C, K132C, D162C, R163C, and E197C.

Variant Selection:

The following 10-Ångstrom variants are considered less preferred due to potential disruption of packing and/or folding: P26C, P28C, Y105C, N106C, and P126C. The following variants are considered less preferred because the corresponding residues define the binding surface for the critical contacts: I110C, F111C, Y127C, and F130C. The remaining 10-angstrom variants were examined for their ability to direct the atoms of a potential tether towards the site of interest, and for the solvent accessibilities of the proposed variants. From this analysis, the preferred variants include E11C, I13C, V16C, K112C, Q113C, V124C, and E129C.

IL-1 R Type 1 Cysteine Variants

Site-directed variants of IL-2 were prepared by the single-stranded DNA method (modification of Kunkel, PNAS, 1985). Oligonucleotides were designed to contain the desired mutations and 15-20 bases of flanking sequence (Table 2).

The single-stranded form of the IL-1R/pRSET plasmid was prepared by transformation of double-stranded plasmid into the CJ236 cell line (1 μl IL-2/pRSET double-stranded DNA, 2 μl 2×KCM salts, 7 μl water, 10 μl PEG-DMSO competent CJ236 cells; incubated at 4 degrees C. for 20 minutes and 25 degrees C. for 10 minutes; plated on LB/agar with 100 μg/ml ampicillin and incubated at 37 degrees C. overnight). Single colonies of CJ236 cells were then grown in 50 ml 2YT media to midlog phase; 5 μl VCS helper phage (Stratagene) were then added and the mixture incubated at 37 degrees C. overnight. Single-stranded DNA was isolated from the supernatant by precipitation of phage (⅕ volume 20% PEG 8000/2.5M NaCl; centrifuge at 12K for 15 min.). Single-stranded DNA was then isolated from phage using Qiagen single-stranded DNA kit.

Site-directed mutagenesis was accomplished as follows: Oligonucleotides (Table 2) were dissolved to a concentration of 10 OD and phosphorylated on the 5′ end (2 μl oligonucleotide, 2 μl 10 mM ATP, 2 μl 10× tris-magnesium chloride buffer, 1 μl 100 mM DTT, 10 μl water, 1 μl T4 PNK; incubate at 37 degrees C. for 45 min.). Phosphorylated oligonucleotides were then annealed to single-stranded DNA template (2 μl single-stranded plasmid, 1 μl oligonucleotide, 1 μl 10×TM buffer, 6 μl water; heat at 94 degrees C. for 2 min., 50 degrees C. for 5 min., cool to room temperature). Double-stranded DNA was then prepared from the annealed oligonucleotide/template (add 2 μl 10×TM buffer, 2 μl 2.5 mM dNTPs, 1 μl 100 mM DTT, 1.5 μl 10 mM ATP, 4 μl water, 0.4 μl T7 DNA polymerase, 0.6 μl T4 DNA ligase; incubate at room temperature for two hours). E. coli (XL1 blue, Stratagene) was then transformed with the double-stranded DNA (1 μl double-stranded DNA, 10 μl 5×KCM, 40 μl water, 50 μl DMSO competent cells; incubate 20 min. at 4 degrees C., 10 min. at room temperature), plated onto LB/agar containing 100 μg/ml ampicillin, and incubated at 37 degrees C. overnight. Approximately four colonies from each plate were used to innoculate 5 ml 2YT containing 100 μg/ml ampicillin; these cultures were grown at 37 degrees C. for 18-24 hours. Plasmids were then isolated from the cultures using Qiagen miniprep kit. These plasmids were sequenced to determine which IL-1R/pRSET clones contained the desired mutation.

TABLE 2
Mutagenic Oligonucleotides for IL-1R type 1

| | |
|---|---|
| Vari- | Mutagenesis Oligonucleotide |
| ant | (5′to 3′) |

| | | |
|---|---|---|
| E11C | TAAAATTATTTTACATTCACGTTCC | (SEQ ID NO: 41) |
| I13C | TGACACTAAAATACATTTTTCTTCACG | (SEQ ID NO: 42) |
| V16C | ATTTGCAGATGAACATAAAATTATTT | (SEQ ID NO: 43) |
| K112C | GGGTAGTTTCTGACAAAATATGGC | (SEQ ID NO: 44) |
| Q113C | AACGGGTAGTTTACACTTAAATATGGC | (SEQ ID NO: 45) |
| V124C | CATATAAGGGCAACAAAGTCCTCC | (SEQ ID NO: 46) |
| E129C | TTTAAAAAAACACATATAAGGGCA | (SEQ ID NO: 47) |

Bold indicates the cysteine anticodon.

Expression of IL-1 R Wild Type and Variant Proteins in Baculovirus

IL1R-FBHT plasmid was site-specifically transposed into the baculovirus shuttle vector (bacmid) by transforming the plasmid into DH10bac (Gibco/BRL) competent cells as follows: 1 μl DNA at 5 ng/μl, 10 μl 5×KCM [0.5 M KCl, 0.15 M CaCl₂, 0.25 M MgCl₂], 30 μl water was mixed with 50 μl PEG-DMSO competent cells, incubated at 4 degrees C. for 20 minutes, 25 degrees C. for 10 minutes, add 900 μl SOC and incubate at 37° C. with shaking for 4 hours, then plated onto LB/agar plates containing 50 μg/ml kanamycin, 7 μg/ml gentamycin, 10 μg/ml tetracycline, 100 μg/ml Bluo-gal, 10 μg/ml IPTG. After incubation at 37° C. for 24 hours, white colonies were grown in 3 ml 2YT media overnight. Cells were then isolated and double-stranded DNA was extracted from the cells as follows: pellet was resuspended in 250 μl of Solution 1 [15 mM Tris-HCl (pH 8.0), 10 mM EDTA, 100 g/ml RNase A]. Add 250 μl of Solution 2 [0.2 N NaOH, 1% SDS] mix gently and incubate at room temperature for 5 minutes. Add 250 μl 3 M potassium acetate, mix and place on ice for 10 minutes. Centrifuge 10 minutes at 14,000×g and transfer supernatant to a tube containing 0.8 ml isopropanol. Mix and place on ice for 10 minutes. Centrifuge 15 minutes at 14,000×g, wash with 70% ethanol, air dry pellet and resuspend DNA in 40 μl TE.

The bacmid DNA was used to transfect Sf9 cells. Sf9 cells were seeded at 9×10⁵ cells per 35 mm well in 2 ml of Sf-900 II SFM medium containing 0.5× concentration of antibiotic-antimycotic and allowed to attach at 27° C. for 1 hour. During this time, 5 μl of IL1R bacmid DNA was diluted into 100 μl of medium without antibiotics, 6 μl of CellFECTIN reagent was diluted into 100 μl of medium without antibiotics and then the 2 solutions were mixed gently and allowed to incubate for 30 minutes at room temperature. The cells were washed once with medium without antibiotics, the medium was aspirated and then 0.8 ml of medium was added to the lipid-DNA complex and overlaid onto the cells. The cells were incubated for 5 hours at 27° C., the transfection medium was removed and 2 ml of medium with antibiotics was added. The cells were incubated for 72 hours at 27° C. and the virus was harvested from the cell culture medium.

The virus was amplified by adding 0.5 ml of virus to a 50 ml culture of Sf9 cells at 2×10⁶ cells/ml and incubating at 27° C. for 48 hours. The virus was harvested from the cell culture medium and this stock was used to express IL1R in High-Five cells. A 1 L culture of High-Five cells at 1×10⁶ cells/ml was infected with virus at an approximate MOI of 2 and incubated for 72 hours. Cells were pelleted by centrifugation and the supernatant was dialyzed overnight against Load buffer (50 mM NaH₂PO₄, pH 8.0; 300 mM NaCL; 10 mM imidazole) at 4° C. The dialyzed supernatant was then loaded onto a Ni-NTA superflow column at 1 ml/min at 4° C., washed with Wash buffer (50 mM NaH₂PO₄, pH 8.0; 300 mM NaCL; 20 mM imidazole) at 2 ml/min, and then eluted from the column with Elution buffer (50 mM NaH₂PO₄, pH 8.0; 300 mM NaCL; 250 mM imidazole) at 2 ml/min collecting 2 ml aliquots. The appropriate fractions were pooled and dialyzed against 3 changes of 4 L of PBS at 4° C. and filtered through a 0.2 μm filter.

### Example 10

### β-Secretase (BACE)

Site of Interest

(B) The site of interest for β-secretase was defined by the crystal structure of the protease domain in complex with a transition-state inhibitor (OM99-2) that spans the S4-S4′ subsites of the enzyme (Hong, L. et al. Science 290, 150-153 (2000)). The residues that compose the site of interest have at least one non-hydrogen atom within 4 Ångstroms of OM99-2. These residues include S10, G11, G12, G13, D32, G34, S35, P70, Y71, T72, Q73, F108, I110, Y198, K224, D228, G230, T231, T232, N233, R235, and T329. Potential 10-Ångstrom variants are defined as all residues of β-secretase within 10 Ångstroms of the site of interest, and which have a side chain solvent accessibility of at least 25% according to the method of Lee and Richards. The list of potential 10-Ångstrom variants includes R7C, K9C, Q12C, H45C, P46C, F47C, H49C, Y68C, P70C, T72C, Q73C, K75C, E77C, T103C, E104C, D106C, K107C, I110C, N111C, A124C, E125C, R128C, P129C, E134C, F159C, P160C, N162C, Q163C, S164C, E165C, L167C, S169C, V170C, P192C, R195C, E196C, W197C, Y198C, K218C, E219C, Y222C, D223C, T232C, N233C, R235C, K238C, K239C, E242C, L263C, E265C, R307C, K321C, S325C, Q326C, S328C, T329C, K350C, V361C, Y384C, and N385C. An additional variant, F108C, is added to this list because it falls within the 10-Ångstrom radius, though its solvent accessibility is less than 25%.

Variant Selection

The following potential 10-Ångstrom variants are considered less preferred because their side chains point away from the substrate binding groove (e.g. the CB atom of the residue is farther from the substrate than the CA atom): R7C, H49C, Y68C, K75C, E77C, T103C, E104C, D106C, N111C, A124C, E134C, F159C, N162C, Q163C, S164C, E165C, S169C, V170C, L176C, E196C, K218C, E219C, Y222C, K238C, K239C, E242C, L263C, E265C, Q326C, V361C, Y384C, and N385C. The following variants are considered less preferred because of potential disruptions in packing or stability: H45C, P46C, F47C, P70C, P129C, P160C, and P192C. The remaining 10-Ångstrom variants were examined for their ability to direct the atoms of a potential tether towards the site of interest. From this analysis, the preferred variants for β-secretase include T72C, Q73C, F108C, I110C, R128C, Y198C, N233C, R235C, and T329C.

### Example 11

HIV Integrase

Site of Interest

Details regarding the exact binding orientation of DNA substrates to HIV integrase are unknown. Mutagenesis studies (Engelmann, A. & Cragie, R. J. Virol. 66, 6361-6369 (1992); Kulkosky, J. et al. Mol. Cell. Biol. 12, 2331-2338 (1992)) have identified residues D64, D116, and E152 of the central core domain as essential for catalysis. A recent crystal structure shows direct contacts between a small molecule inhibitor of HIV integrase and residues T66, Q148, E152, N155, K156, and K159 of the core domain (Goldgur, Y. et al. Proc. Natl. Acad. Sci. USA 96, 13040-13043 (1999)). Docking studies of known integrase inhibitors have proposed a binding site consisting of residues D64, C65, T66, H67, Q92, D116, Q148, I151, E152, N155, K156, and K159 (Sotriffer, C. A. et al. J. Med. Chem. 43, 4109-4117 (2000)). In addition, the residues G140, I141, P142, Y143, N144, P145, Q146, S147, and G149 of a nearby flexible loop have been implicated in catalysis (Greenwald, J. et al. Biochemistry 38, 8892-8898 (1999)). From this information, the site of interest is defined as residues D64, C65, T66, H67, Q92, D116, G140, I141, P142, Y143, N144, P145, Q146, S147, Q148, I151, E152, N155, K156, and K159. Potential 10-Ångstrom variants are defined as all residues of δHIV integrase within 10 Ångstroms of the site of interest, and which have a side chain solvent accessibility of at least 25% according to the method of Lee and Richards. Distance measurements were based on the structure reported by Maignan et al. (PDB code 1BL3) (Maignan, S. et al. J Mol Biol 282, 359-368 (1998)). The third copy in the asymmetric unit (chain C) was used for all measurements. The list of potential 10-Ångstrom variants includes M50C, Q53C, V54C, D55C, 160C, D64C, H67C, L68C, E69C, K71C, E87C, P90C, A91C, E92C, T93C, Q95C, E96C, H114C, D116C, N117C, S119C, T122C, S123C, K127C, K136C, E138C, F139C, P142C, Y143C, N144C, P145C, Q146C, S147C, Q148C, E152C, S153C, K156C, K159C, K160C, Q164C, R166C, and M178C. Residue Q62 sits under a loop that is disordered in most structures, and may be solvent-exposed. Residue N120 becomes more solvent-exposed in structures co-crystallized with cacodylate (Goldgur, Y. et al. Proc. Natl. Acad. Sci. USA 95, 9150-9154 (1998)). Residues Q62 and N120 are therefore included as potential 10-Ångstrom variants. These variants are preferably introduced against a background where naturally occurring cysteines C56, C65, and C130 are mutated to any combination of alanine or serine. Residues F185 or W131 may also be mutated to a more polar residue such as ARG, LYS, HIS, ASN, ASP, GLN, or GLU to improve protein solubility.

Variant Selection

The following 10-Ångstrom variants are considered less preferred because their side chains point away from the site of interest (e.g. the CB atom of the residue is farther from the site of interest than the CA atom): K71C, E87C, A91C, T93C, Q95C, E96C, K136C, E138C, S153C, K160C, Q164C, M178C. The following 10-Ångstrom variants are considered less preferred because replacement may affect protein packing or stability: 160C, E69C, P90C, F139C, R166C. The following 10-Ångstrom variants are considered less preferred because they occur in flexible regions that are poorly-defined across a number of crystal structures: M50C, Q53C, V54C, D55C, P142C, Y143C, N144C, P145C, Q146C, S147C. The remaining 10-Ångstrom variants were examined for their ability to direct the atoms of a potential tether towards the site of interest, and for the solvent accessibilities of the proposed variants. From this analysis, the preferred variants for HIV integrase include Q62C, H67C, E92C, D116C, N120C, H114C, E152C, and K156C. Wild-type cysteine C165 is also preferred as a tethering site, provided that the remaining cysteines (C56 and C130) are mutated to alanine or serine.

### Example 12

### Tumor Necrosis Factor-Alpha (TNF-α)

Site of Interest

There are no published crystal structures of the complexes between TNF-α and its two primary receptors, p55 and p75. Models of these two complexes have been proposed (Fu, Z.-Q. et al. Protein Engineering 8, 1233-1241 (1995)) based on crystal structures of TNF-α (PDB code 1TNF) (Eck. M. J. & Sprang, S. R. J. Biol. Chem. 264, 17595-17605 (1989)) and a co-crystallized structure of the complex between TNF-β and the p55 receptor (PDB code 1TNR) (Banner, D. W. et al. Cell 73, 431-445 (1993)). These models suggest that the two receptors share overlapping binding sites on TNF-α the p55 receptor is proposed to contact residues R6, Q21, Q23, R31, R32, A33, K65, L75, S86, Y87, P113, Y115, D140, D143, F144, A145, and Q149, while the p75 receptor is proposed to contact residues Q21, R31, R32, A33, K65, Q67, V74, Y87, P113, and A145. Domain 4 of p75 has also been implicated in binding to TNF-α, (Chen, P. C.-H. et al. J. Biol. Chem. 270, 2874-2878 (1995)) but this domain was not included in the model. Mutagenesis studies (Loetscher, H. et al. J. Biol. Chem. 268, 26350-26357 (1993); Van Ostade, X. et al. EMBO J. 10, 827-836 (1991); Zhang, X.-M. et al. J. Biol. Chem. 267, 24069-24075 (1992); Chih-Hsuch, P. et al. J. Biol. Chem. 270, 2874-2878 (1995)) implicate residues R32, A33, S86, Y87, V91, S95, D143, A145, and S147 as important for binding to p55 and/or p75. Of these positions, Y87, which is located at the apex of a flexible loop, appears to be essential for binding to both receptors.

When mapped onto the crystal structure, these residues encircle a cleft between the subunits of the TNF-α trimer, and are consistent with the models described above. This same cleft is occupied by the p55 receptor in the TNF-β complex. Based on these observations, the site of interest for TNF-α is defined by residues R32, A33, S86, Y87, V91, S95, D143, A145, and S147. Potential 10-Angstrom variants are defined as all residues on the TNF-α trimer within 10 Ångstroms of the site of interest, and which have a side chain solvent accessibility of at least 25% according to the method of Lee and Richards. Distance measurements were based on the structure reported by Eck & Sprang, as well as a proprietary structure of TNF-α. Any residue of either structure that had at least 25% side chain solvent accessibility in all 3 monomers of that structure was considered accessible. Based on this definition, the potential 10-Ångstrom variants include: N19C, P20C, Q21C, Q25C, Q27C, N30C, R31C, R32C, A33C, K65C, Q67C, L75C, T77C, T79C, A84C, V85C, S86C, Y87C, Q88C, T89C, K90C, V91C, N92C, P113C, Q125C, E127C, K128C, E135C, R138C, P139C, D140C, A145C, E146C, and S147C.

Variant Selection:

The following 10-Ångstrom variants are considered less preferred their side chains point away from the site of interest (e.g. the CB atom of the residue is farther from the site of interest than the CA atom): N19C, Q21C, Q25C, Q27C, N30C, R31C, K65C, Q67C, E127C, K128C, R138C, P139C, and D140C. The following 10-Ångstrom variants are considered less preferred due to their location on a flexible loop that shows variability across crystal structures: V85C, S86C, Y87C, and Q88C. 10-Ångstrom variants are considered less preferred because mutation to cysteine may affect folding or protein stability: P20C, K90C, P114C, and E135C. The remaining 10-Ångstrom variants were examined for their ability to direct the atoms of a potential tether towards the site of interest, and for the solvent accessibilities of the proposed variants. From this analysis, the preferred variants for TNF-α include R32C, A33C, T77C, V91C, Q125C, and S147C.

### Example 13

### Truncated Wild Type Human PTP-1B

Cloning of PTP-1B

A cDNA encoding the first 321 amino acids of human PTP-1B was isolated from human fetal heart total RNA (Clontech). Oligonucleotide primers corresponding to nucleotides 91 to 114 (For) and complementary to nucleotides 1030 to 1053 (Rev) of the PTP-1B cDNA (Genbank M31724.1, Chemroff, 1990) were synthesized and used to generate a DNA using the polymerase chain reaction.

Forward:

(SEQ ID NO:48)

GCC CAT ATG GAG ATG GAA AAG GAG TTC GAG
Reverse:

(SEQ ID NO:49)

GCG ACG CGA ATT CTT AAT TGT GTG GCT CCA GGA TTC G
TTT

The primer Forward incorporates an NdeI restriction site at the first ATG codon and the primer Rev inserts a UAA stop codon followed by an EcoRI restriction site after nucleotide 1053. cDNAs were digested with restriction nucleases NdeI and EcoRI and cloned into pRSETc (Invitrogen) using standard molecular biology techniques. The identity of the isolated cDNA was verified by DNA sequence analysis (methodology if outlined in a later paragraph).

A shorter cDNA, PTP-1B 298, encoding amino acid residues 1-298 was generated using oligonucleotide primers Forward and Rev2 and the clone described above as a template in a polymerase chain reaction.

Rev2:

(SEQ ID NO:50)

5′-TGC CGG AAT TCC TTA GTC CTC GTG GGA AAG CTC C

PTP-1B Mutants

Site-directed mutants of PTP-1B (amino acids 1-321), PTP-1B 298 (amino acids 1-298) and PTP-1B 298-2M (with Cys32 and Cys92 changed to Ser and Val, respectively) were prepared by the single-stranded DNA method (modification of Kunkel, 1985). Oligonucleotides were designed to contain the desired mutations and 12 bases of flanking sequence on each side of the mutation.

The single-stranded form of the PTP-1B/pRSET, PTP-1B 298/pRSET and PTP-1B 298-2M/pRSET plasmid was prepared by transformation of double-stranded plasmid into the CJ236 cell line (1 μl double-stranded plasmid DNA, 2 μl 5×KCM salts, 7 μl water, 10 μl PEG-DMSO competent CJ236 cells; incubated on ice for 20 minutes followed by 25° C. for 10 minutes; plated on LB/agar with 100 μg/ml ampicillin and incubated at 37° C. overnight). Single colonies of CJ236 cells were then grown in 100 ml 2YT media to midlog phase; 5 μl VCS helper phage (Stratagene) were then added and the mixture incubated at 37° C. overnight. Single-stranded DNA was isolated from the supernatant by precipitation of phage (⅕ volume 20% PEG 8000/2.5M NaCl; centrifuge at 12K for 15 min.). Single-stranded DNA was then isolated from phage using Qiagen single-stranded DNA kit.

Site-directed mutagenesis was accomplished as follows: Oligonucleotides were dissolved in TE (10 mM Tris pH 8.0, 1 mM EDTA) to a concentration of 100D and phosphorylated on the 5′ end (2 μl oligonucleotide, 2 μl 10 mM ATP, 2 μl 10× tris-magnesium chloride buffer, 1 μl 100 mM DTT, 12.5 μl water, 0.5 μl T4 PNK; incubate at 37 degrees C. for 30 min.). Phosphorylated oligonucleotides were then annealed to single-stranded DNA template (2 μl single-stranded plasmid, 0.6 μl oligonucleotide, 6.4 μl water; heat at 94 degrees C. for 2 min., slow cool to room temperature). Double-stranded DNA was then prepared from the annealed oligonucleotide/template (add 2 μl 10×TM buffer, 2 μl 2.5 mM dNTPs, 1 μl 100 mM DTT, 0.5 μl 10 mM ATP, 4.6 μl water, 0.4 μl T7 DNA polymerase, 0.2 μl T4 DNA ligase; incubate at room temperature for two hours). E. coli (XL1 blue, Stratagene) were then transformed with the double-stranded DNA (5 μl double-stranded DNA, 5 μl 5×KCM, 15 μl water, 25 μl PEG-DMSO competent cells; incubate 20 min. on ice, 10 min. at room temperature), plated onto LB/agar containing 100 μg/ml ampicillin, and incubated at 37 degrees C. overnight. Approximately four colonies from each plate were used to innoculate 5 ml 2YT containing 100 μg/ml ampicillin; these cultures were grown at 37 degrees C. for 18-24 hours. Plasmids were then isolated from the cultures using Qiagen miniprep kit. These plasmids were sequenced to determine which clones contained the desired mutation.

Mutagenesis oligonucleotides

| | | |
|---|---|---|
| A27C | 5′- tgggaagtcactgcattcatgtc | (SEQ ID NO:51) |
| | ggat | |
| C215S | 5′- gatgcctgcactggagtgcacca | (SEQ ID NO:52) |
| | caac | |
| C32S | 5′- cttggccactctagatgggaagt | (SEQ ID NO:53) |
| | cact | |
| C92V | 5′- ccaaaagtgaccgactgtgttag | (SEQ ID NO:54) |
| | gcaa | |
| D181C | 5′- agggactccaaagcaaggccatg | (SEQ ID NO:55) |
| | tggt | |
| D29C | 5′- tctacatgggaagcaactggctt | (SEQ ID NO:56) |
| | catg | |
| D29C-2M | 5′- tctagatgggaagcaactggctt | (SEQ ID NO:57) |
| | catg | |
| D48C | 5′- aaagggactgacgcatctgtacc | (SEQ ID NO:58) |
| | tatt | |
| F182C | 5′- ttcagggactccacagtcaggcc | (SEQ ID NO:59) |
| | atgt | |
| F52C | 5′- ccgactatggtcacagggactga | (SEQ ID NO:60) |
| | cgtc | |
| H25C | 5′- gtcactggcttcacatcggatat | (SEQ ID NO:61) |
| | cctg | |
| K120C | 5′- gtattgtgcgcaacataacgaac | (SEQ ID NO:62) |
| | cttt | |
| K36C | 5′- gttcttaggaagacaggccactc | (SEQ ID NO:63) |
| | taca | |
| K36C-2M | 5′- gttcttaggaagacaggccactc | (SEQ ID NO:64) |
| | taga | |
| M258C | 5′- ctggatcagcccacaccgaaact | (SEQ ID NO:65) |
| | tcct | |
| Q262C | 5′- ctggtcggctgtacagatcagcc | (SEQ ID NO:66) |
| | ccat | |
| R47C | 5′- gggactgacgtcacagtacctat | (SEQ ID NO:67) |
| | ttcg | |
| S50C | 5′- atggtcaaagggacagacgtctc | (SEQ ID NO:68) |
| | tgta | |
| V49C | 5′- gtcaaagggactgcagtctctgt | (SEQ ID NO:69) |
| | acct | |
| Y46C | 5′- actgacgtctctgcacctatttc | (SEQ ID NO:70) |
| | ggtt | |

Sequencing of PTP-1B clones was accomplished as follows: The concentration of plasmid DNA was quantitated by absorbance at 280 nm. 1000 ng of plasmid was mixed with sequencing reagents (1 μg DNA, 6 μl water, 1 μl sequencing primer at 3.2 pm/μl, 8 μl sequencing mixture with Big Dye [Applied Biosystems]). The sequencing primers are T7 FOR 5′-aatacgactcactatag (SEQ ID NO:71) and RSET REV 5′-tagttattgctcagcggtgg (SEQ ID NO:72). The mixture was then run through a PCR cycle (96 degrees, 10 s; 50 degrees, 5 s; 60 degrees 4 min; 25 cycles) and the DNA reaction products were precipitated (20 μl mixture, 80 μl 75% isopropanol; incubated 20 min at room temperature then pelleted at 14 K rpm for 20 min; wash with 250 μl 75% isopropanol; heat 1 min at 94 degrees C.). The precipitated products were then resuspended in 20 μl TSB (Applied Biosystems) and the sequence read and analyzed by an Applied Biosystems 310 capillary gel sequencer. In general, ¼ of the plasmids contained the desired mutation.

Expression of Truncations and Cys Mutants of PTP-1B

Mutant proteins were expressed as follows: PTP-1B clones were transformed into BL21 codon plus cells (Stratagene) (1 μl double-stranded DNA, 2 μl 5×KCM, 7 μl water, 10 μl DMSO competent cells; incubate 20 min. at 4 degrees C., 10 min. at room temperature), plated onto LB/agar containing 100 μg/ml ampicillin, and incubated at 37 degrees C. overnight. 2 single colonies were picked off the plates or from frozen glycerol stocks of these mutants and inoculated in 100 ml 2YT with 50 μg/ml carbenicillin and grown overnight at 37 degrees C. 50 ml from the overnight cultures were added to 1.5 L of 2YT/carbenicillin (50 μg/ml) and incubated at 37 degrees C. for 3-4 hours until late-log phase (absorbance at 600 nm ˜0.8-0.9). At this point, protein expression was induced with the addition of IPTG to a final concentration of 1 mM. Cultures were incubated at 37 degrees for another 4 hours and then cells were harvested by centrifugation (7 Krpm, 7 min) and frozen at −20 degrees C.

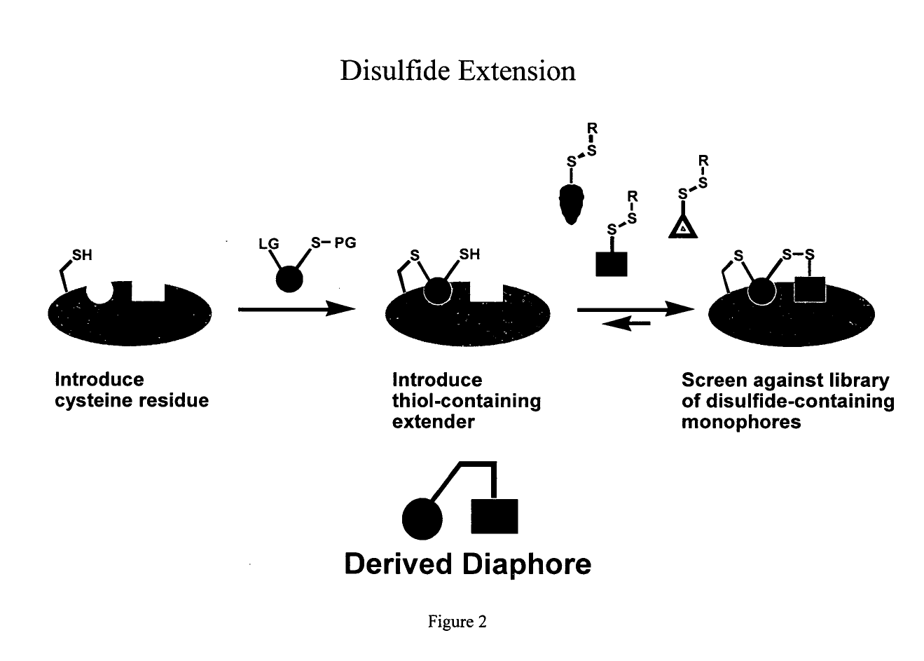

PTP-1B proteins were purified from the frozen cell pellets as described in the following: First, cells were lysed in a microfluidizer in 100 ml of buffer containing 20 mM MES pH 6.5, 1 mM EDTA, 1 mM DTT, and 10% glycerol buffer (with 3 passes through a Microfluidizer [Microfluidics 110S]) and inclusion bodies were removed by centrifugation (10 Krpm, 10 min.). Purification of all PTP-1B mutants was performed at 4 degrees C. The supernatants from the centrifugation were filtered through 0.45 μm cellulose acetate (5 μl of this material was analyzed by SDS-PAGE, FIG. 2) and loaded onto an SP Sepharose fast flow column (2.5 cm diameter×14 cm long) equilibrated in Buffer A (20 mM MES pH 6.5, 1 mM EDTA, 1 mM DTT, 1% glycerol) at 4 ml/min.

Thus, as shown in FIG. 2, in the first step, a target molecule containing or modified to contain a free thiol group (such as a cysteine-containing protein) is modified by a thiol-containing extender, comprising a reactive group capable of reacting with the thiol group on the target molecule, a portion having intrinsic affinity for the target molecule, and a thiol group. The complex formed between the target molecule and the thiol-containing extender is then used to screen a library of disulfide-containing monophores to identify a library member that has the highest intrinsic binding affinity for a second binding site on the target molecule.

The protein was then eluted using a gradient of 0-50% Buffer B over 60 minutes (Buffer B: 20 mM MES pH 6.5, 1 mM EDTA, 1 mM DTT, 1% glycerol, 1 M NaCl). Yield and purity was examined by SDS-PAGE and, if necessary, PTP-1B was further purified by hydrophobic interaction chromatography (HIC): Protein was supplemented with ammonium sulfate until a final concentration of 1.4 M was reached. The protein solution was filtered and loaded onto an HIC column at 4 ml/min in Buffer A2: 25 mM Tris pH 7.5, 1 mM EDTA, 1.4 M (NH₄)₂SO₄, 1 mM DTT. Protein was eluted with a gradient of 0-100% Buffer B over 30 minutes (Buffer B2: mM Tris pH 7.5, 1 mM EDTA, 1 mM DTT, 1% glycerol). Finally, the purified protein was dialyzed at 4 degrees C. into the appropriate assay buffer (25 mM Tris pH 8, 100 mM NaCl, 5 mM EDTA, 1 mM DTT, 1% glycerol). Yields varied from mutant to mutant but typically were within the range of 3-20 mg per 1-liter culture.

### Example 14

### Human Interleukin-4

IL-4 Cloning

The DNA sequence encoding human interleukin-4 (IL4) was isolated by PCR from the plasmid pcD-hIL-4 (ATCC Accession No. 57592) using PCR primers:

| | |
|---|---|
| IL4 ForRse | |
| 5′- gggtttcatatgcacaagtgcgatatcacctt | (SEQ ID NO:73) |
| IL4 RevRse | |
| 5′- ccgctcgagtcagctcgaacactttgaata | (SEQ ID NO:74) |

These primers correspond to extracellular domain of the protein and which were designed to contain restriction endonuclease sites Nde I and XhoI for subcloning into a pRSET vector (Invitrogen). The PCR reaction was purified on a Qiaquick PCR purification column (Qiagen). The PCR product containing the IL4 sequence was cut with restriction endonucleases (41 μl PCR product, 2 μl each endonuclease, 5 μl appropriate 10× buffer; incubated at 37° C. for 90 minutes). The pRSET vector was cut with restriction endonucleases (6 μg DNA, 4 μl each endonuclease, 10 μl appropriate 10× buffer, water to 100 μl; incubated at 37° C. for 2 hours; add 2 μl CIP and incubated at 37° C. for 45 minutes). The products of nuclease cleavage were isolated from an agarose gel (1% agarose, TBE buffer) and ligated together using T4 DNA ligase (200 ng pRSET vector, 150 ng IL4 PCR product, 4 μl 5× ligase buffer [300 mM Tris pH7.5, 50 mM MgCl₂, 20% PEG 8000, 5 mM ATP, 5 mM DTT], 1 μl ligase; incubated at 15° C. for 1 hour). 10 μl of the ligation reaction was transformed into XL1 blue cells (Stratagene) (10 μl reaction mixture, 10 μl 5×KCM [0.5 M KCl, 0.15 M CaCl₂, 0.25 M MgCl₂], 30 μl water, 50 μl PEG-DMSO competent cells; incubated at 4 degrees C. for 20 minutes, 25 degrees C. for 10 minutes), and plated onto LB/agar plates containing 100 μg/ml ampicillin. After incubation at 37 degrees overnight, single colonies were grown in 3 ml 2YT media for 18 hours. Cells were then isolated and double-stranded DNA extracted from the cells using a Qiagen DNA miniprep kit.

IL-4 Sequence and Numbering System

The human IL-4 sequence shown in the upper row (SEQ ID NO: 86) is from published cDNA sequences and includes the secretion signal sequence (Yokota, T. et al., PNAS 83 16 5894-5898). The lower row (SEQ ID NO: 87) shows the IL-4 sequence that lacks the secretion signal and contains an additional N-terminal methionine, expressed intracellularly in E. coli from the Sunesis RSET.IL4 plasmid

```
1 24 53
| | |
MGLTSQLLPPLFFLLACAGNFVHG HKCDITLQE IIKTLNSLTE QKTLCTELTV
                                                MHKCDITLQE IIKTLNSLTE QKTLCTELTV
                                                | |
                                                1 30
  54 103
    | |
    TDIFAASKNT TEKETFCRAA TVLRQFYSHH EKDTRCLGAT AQQFHRHKQL
    TDIFAASKNT TEKETFCRAA TVLRQFYSHH EKDTRCLGAT AQQFHRHKQL
    | |
    31 80
  104 153
    | |
    IRFLKRLDRN LWGLAGLNSC PVKEANQSTL ENFLERLKTI MREKYSKCSS
    IRFLKRLDRN LWGLAGLNSC PVKEANQSTL ENFLERLKTI MREKYSKCSS
    | |
  81 130
```

Generation of IL-4 Cysteine Mutations

Mutations were generated using as previously described (Kunkel et al., Methods Enzymol. 154:367-82 (1987). DNA oligonucleotides used are shown below and were designed to hybridize with sense strand DNA from plasmid.

| | oligonucleotide sequence | |
|---|---|---|
| mutation | (5′ to 3′) | SEQ ID NO: |
| E9C | AGTTTTGATGATACACTGTAAGGTGAT | 75 |
| K102C | CTGGTTGGCTTCACACACAGGACAGG | 76 |
| K117C | CTCTCATGATCGTGCATAGCCTTTCC | 77 |
| K12C | GCTGTTCAAAGTGCAGATGATCTCCTG | 78 |
| K37C | CAGTTGTGTTACAGGAGGCAGCAAAG | 79 |
| K42C | GCAGAAGGTTTCACACTCAGTTGTG | 80 |
| N38C | CCTTCTCAGTTGTGCACTTGGAGGC | 81 |
| N97C | CACAGGACAGGAACACAAGCCCGCC | 82 |
| Q54C | GGCTGTAGAAACACCGGAGCACAGTCG | 83 |
| R121C | GAATATTTCTCACACATGATCGTC | 84 |
| S16C | CTGCTCTGTGAGGCAGTTCAAAGT | 85 |

IL-4 Expression/Purification

a. Fermentation

1. Start overnight culture (BL21 DE3 transformed with RSET.IL4),
2. 30 mL 2YT+50 μg/ml ampicillin
3. Use overnight culture to inoculate 1.5 L culture 2YT+50 μg/ml ampicillin

### 4. Grow to OD600˜0.8

5. Induce with 1 mM IPTG for 4 hr
6. Spin cells down, 7000 rpm/10 min, freeze −20 degrees

b. Protein Extraction

### 1. Resuspend in 100 mL of TE+50 mM NaCl

2. Run through the microfluidizer 2×
3. Spin down, 7000 rpm/15 min
4. Wash pellet w/50 mL TE/NaCl
5. Spin down 7000 rpm/15 min

### 6. Resuspend Pellet in 50 ml; 5 M Gdn-HCl

50 mM Tris, pH 8
50 mM NaCl
50 mM Reduced Glutathione

Incubate RT, 1 hr, should become clear

Spin down 7500 rpm/15 min, filter

c. Refold

Add slowly (with stirring) to 9 volumes (450 mL);
50 mM Tris pH 8
50 mM NaCl
0.2 mM GSSG

Incubate with slow stirring for 3 hours at ambient temperature

Dialyze 3 times in MWCO 3000 dialysis bag vs 0.5×PBS (20 liters)

d. Purification:

Hi-S Column Cartridge (Bio-Rad)

Buffer A: 0.5×PBS

Buffer B: PBS+1 M NaCl

Load column at 5 ml/min, Wash with buffer A for 15-20 min, Collect 7.5 ml fractions over 0-100% B gradient for 60 minutes at 5 ml/min.

Concentrate with 5 k mwco filter, buffer exchange with PBS, 0.2 micron filter and store at −80 degrees.

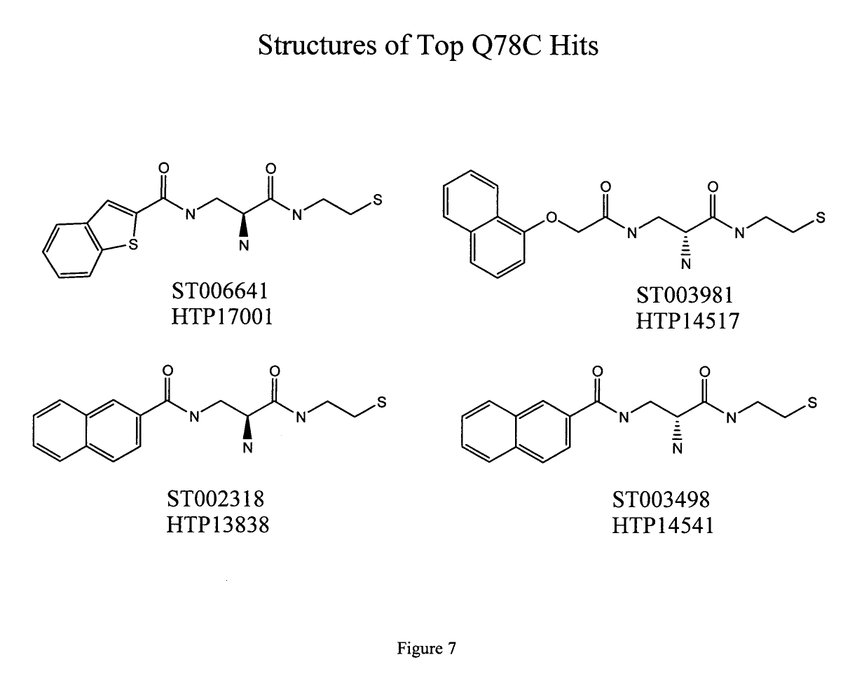

As shown in FIG. 7, the following ligands were shown to bind with the Q78C variant of human IL-4: ST006641, ST002318, ST003981 and ST003498.

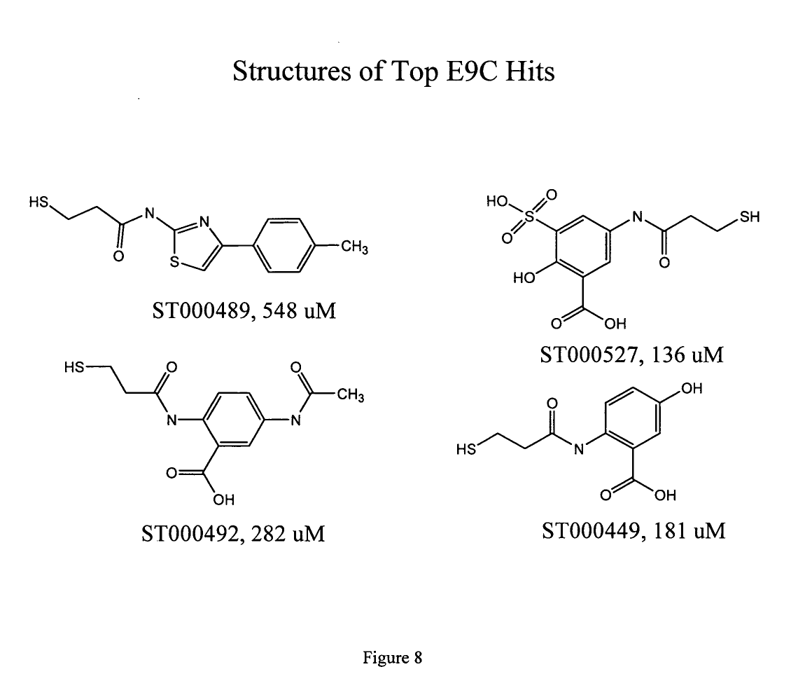

As illustrated in FIG. 8, the following ligands were found to bind with the E9C variant of human IL-4: ST000489 at 548 μM, ST000527 at 136 μM, ST000492 at 282 μM and ST000449 at 181 μM

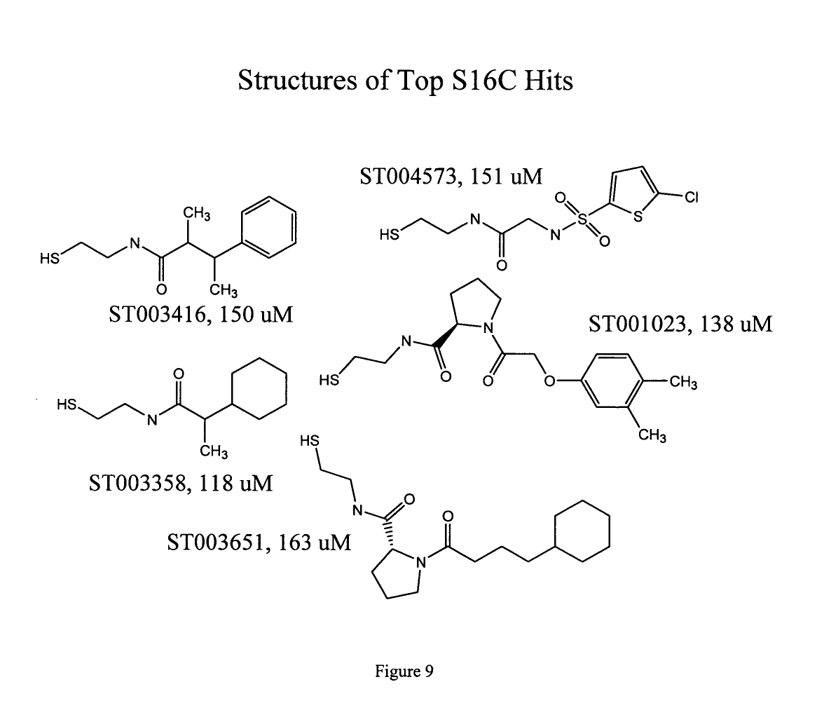

As shown in FIG. 9, the following ligands were found to bind with the S16C variant of human IL-4: ST003416 at 150 μM, ST003358 at 118 μM, ST004573 at 151 μM, ST001023 at 138 μM and ST003651 at 163 μM.

Fraction Tethered

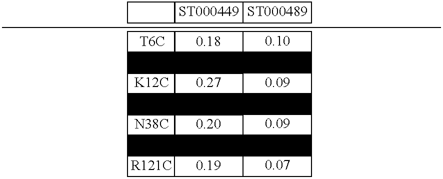

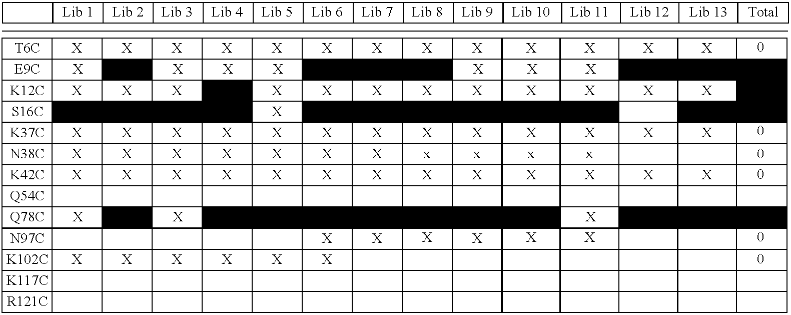

### CONCLUSION

All references cited throughout the specification are hereby expressly incorporated by reference.

While the present invention has been described with reference to the specific embodiments thereof, it would be understood by those skilled in the art that various changes may be made and equivalents may be substituted without departing from the true spirit and scope of the invention. In addition, many modifications may be made to adapt a particular situation, material, composition of matter, process, process step or steps, to the objection, spirit and scope of the present invention. All such modifications are intended to be within the scope of the claims appended hereto.

1-68. (canceled)

69. A library of compounds comprising a plurality of compounds of Structural Formula II 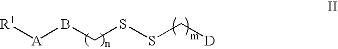
where
A is absent or is selected from 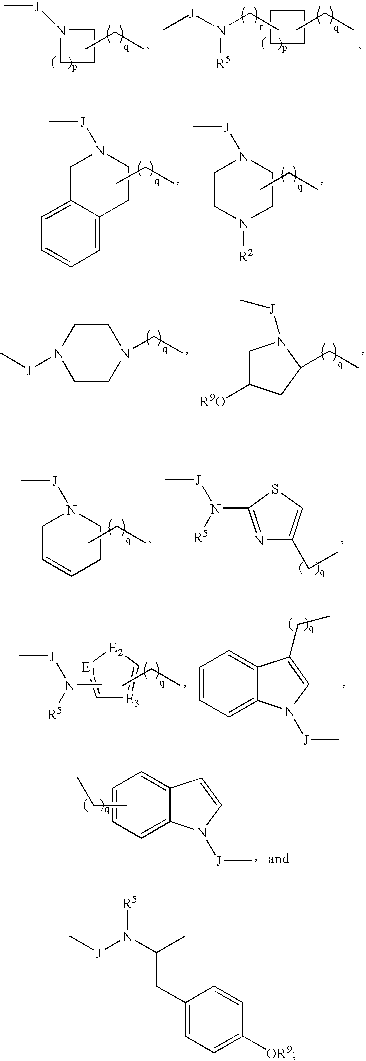
B is selected from
—C(═O)—NH—, and
—NR⁵—C(═O)—;
D is selected from
—NH₂,
—NHCOCH₃,
—NHCONH₂,
—NHCH₃,
—N(CH₃)₂, and
—N(CH₃)₃;
E₁, E₂, and E₃ are independently selected from NH, N, CH₂ and CH;
J is absent or is selected from
hydrogen,
OH,
—C(═O)—,
—NH—C(═O)—,
—SO₂—,
—NH—SO₂—, and
—NH—C(═S)—;
m and n are integers selected from 2 to 4;
p is an integer selected from 1 to 3;
q and r are integers selected from 0 to 2;
R¹ is selected from the group
hydrogen,
C₁-C₆alkyl,
C₀-C₄alkyl-C₃-C₁₁cycloalkyl C₀-C₄alkyl-C₃-C₆cycloalkyl-C₆-C₁₂aryl,
C₀-C₄alkyl-C₆-C₁₂aryl, and
C₀-C₄alkyl-het,
where
het is a heterocycle group composed of from 1 to 3 rings fused or linked sequentially, any ring independently being a saturated or unsaturated homocycle or heterocycle or a homo- or heteroaromatic, each ring containing 4, 5, 6 or 7 ring atoms and from 0-4 heteroatoms selected from N, O and S, provided at least one ring contains a heteroatom, where any homocycle or heterocycle ring N, S or C may optionally be oxidized and where any ring nitrogen may be substituted with R⁵ or R¹;
cycloalkyl may be a mono-, bi- or tricycle, where any nonadjacent cyclo-carbon may be oxidized to a ketone and where the cycloalkyl may optionally be fused to a C₆-C₁₂aryl, and
where
any alkyl, cycloalkyl or heterocycle may be substituted with 1-3 R³ and any aryl or heteroaryl may be substituted with 1-3 R⁴;
R³ and R⁴ are independently selected from
C₁-C₆alkyl,
C₁-C₆alkyloxy-C₀-C₆alkyl,
C₁-C₆alkylthio-C₀-C₆alkyl,
C₁-C₆alkylcarbonyl-C₀-C₆alkyl,
C₁-C₄alkylamino-C₀-C₆alkyl,
C₁-C₆alkylcarbonylamino-C₀-C₆alkyl,
C₁-C₆alkyloxycarbonyl-C₀-C₆alkyl,
C₁-C₆alkyloxycarbonyl-C₁-C₆alkyloxy,
C₁-C₄alkylcarbonylaminocarbonyl-C₀-C₆alkyl,
C₁-C₄alkylcarbonyloxy-C₀-C₆alkyl,
C₁-C₄alkylcarbonylaminosulfonyl-C₀-C₆alkyl
C₁-C₄alkylaminocarbonyloxy-C₀-C₆alkyl,
C₁-C₄alkylaminocarbonyl-C₀-C₆alkyl,
C₁-C₄alkylaminocarbonylamino-C₀-C₆alkyl,
C₁-C₄alkylaminothiocarbonylamino-C₀-C₆alkyl,
C₁-C₄alkylaminocarbonylaminosulfonyl-C₀-C₆alkyl,
C₁-C₄alkylsulfonylaminocarbonylamino-C₀-C₆alkyl,
C₁-C₄alkylsulfonylaminocarbonyl-C₀-C₆alkyl,
di-(C₁-C₄alkyl)amino-C₁-C₄alkylaminocarbonyl,
di-(C₁-C₄alkyl)amino-sulfonyl,
C₁-C₄alkylaminosulfonyl-C₁-C₄alkyl,
C₁-C₄alkyloxycarbonylamino-C₀-C₆alkyl,
C₁-C₄alkyloxime,
C₁-C₄alkylsulfonyl,
C₃-C₁₁ cycloalkyl,
C₃-C₁₁ cycloalkylamino,
C₃-C₁₁ cycloalkylcarbonyl,
C₃-C₁₁ cycloalkyloxy,
C₃-C₁₁ cycloalkylthio,
aminosulfonyl,
aminocarbonyl,
aminocarbonyl-C₁-C₄alkyl,
aminocarbonyl-C₁-C₄alkyloxy,
amino-C₃-C₇ cycloalkyl,
C₆-C₁₂ aryl-C₀-C₆alkyl,
C₆-C₁₂ aryloxy-C₀-C₆alkyl,
C₆-C₁₂ arylthio-C₀-C₆alkyl,
C₆-C₁₂ arylcarbonyl-C₀-C₆alkyl,
C₆-C₁₂ arylamino-C₀-C₆alkyl,
C₆-C₁₂ arylcarbonylamino-C₀-C₆alkyl,
C₆-C₁₂ aryloxycarbonyl-C₀-C₆alkyl,
C₆-C₁₂ aryloxycarbonyl-C₁-C₆alkyloxy,
C₆-C₁₂ arylcarbonylaminocarbonyl-C₀-C₆alkyl,
C₆-C₁₂ arylcarbonyl-C₁-C₆alkyloxycarbonyl-C₀-C₆alkyl,
C₆-C₁₂ arylcarbonyl-C₁-C₆alkyloxy-C₀-C₆alkyl,
C₆-C₁₂ arylcarbonylaminosulfonyl-C₀-C₆alkyl,
C₆-C₁₂ arylcarbonyloxy-C₀-C₆alkyl,
C₆-C₁₂ arylaminocarbonyloxy-C₀-C₆alkyl,
C₆-C₁₂ arylaminocarbonyl-C₀-C₆alkyl,
C₆-C₁₂ arylaminocarbonylamino-C₀-C₆alkyl,
C₆-C₁₂ arylaminothiocarbonylamino-C₀-C₆alkyl,
C₆-C₁₂ arylaminocarbonylaminosulfonyl-C₀-C₆alkyl,
C₆-C₁₂ arylsulfonylaminocarbonylamino-C₀-C₆alkyl,
C₆-C₁₂ arylsulfonylaminocarbonyl-C₀-C₆alkyl,
di-(C₆-C₁₂ aryl)amino-C₁-C₄alkylaminocarbonyl,
C₆-C₁₂ arylaminosulfonyl-C₀-C₄alkyl,
C₆-C₁₂ aryloxycarbonylamino-C₀-C₆alkyl,
C₆-C₁₂ aryloxime,
C₆-C₁₂ aryl-C₁-C₄alkylaminocarbonyl-C₀-C₆alkyl,
C₆-C₁₂ arylsulfonyl-C₀-C₆alkyl,
C₆-C₁₂ aryl-C₁-C₄alkylsulfonyl-C₀-C₆alkyl,
C₆-C₁₂ aryl-C₁-C₄alkyloxy-C₀-C₆alkyl,
C₆-C₁₂ aryl-C₁-C₄alkyloxycarbonyl-C₀-C₆alkyl,
C₆-C₁₂ aryl-C₁-C₄alkyloxycarbonylamino-C₀-C₆alkyl, carboxy-C₁-C₄alkyloxy,
carboxy-C₁-C₄alkyl,
carboxy-C₁-C₄alkylaminocarbonyl,
carboxy-C₁-C₄alkylthio,
carbonyl-C₁-C₄alkyl,
cyano-C₁-C₄alkyl,
cyano-C₁-C₄alkylaminosulfonyl,
het-C₀-C₆alkyl,
het-oxy-C₀-C₆alkyl,
het-thio-C₀-C₆alkyl,
het-carbonyl-C₀-C₆alkyl,
het-amino-C₀-C₆alkyl,
het-carbonylamino-C₀-C₆alkyl,
het-oxycarbonyl-C₀-C₆alkyl,
het-oxycarbonyl-C₁-C₆alkyloxy,
het-carbonylaminocarbonyl-C₀-C₆alkyl,
het-carbonylaminosulfonyl-C₀-C₆alkyl,
het-carbonyloxy-C₀-C₆alkyl,
het-aminocarbonyloxy-C₀-C₆alkyl,
het-aminocarbonyl-C₀-C₆alkyl,
het-aminocarbonylamino-C₀-C₆alkyl,
het-aminothiocarbonyl amino-C₀-C₆alkyl,
het-aminocarbonylaminosulfonyl-C₀-C₆alkyl,
het-sulfonylaminocarbonylamino-C₀-C₆alkyl,
het-sulfonylaminocarbonyl-C₀-C₆alkyl,
(het),(C₆-C₁₂ aryl)amino-C₁-C₄alkylaminocarbonyl,
het-aminosulfonyl-C₀-C₄alkyl,
het-oxycarbonylamino-C₀-C₆alkyl,
het-oxime,
het-C₁-C₄alkylaminocarbonyl,
het-sulfonyl,
het-C₁-C₄alkyloxycarbonyl,
het-C₁-C₄alkyloxycarbonylamino,
hydroxy-C₁-C₆alkylcarbonyl,
hydroxy-C₁-C₆alkyl,
hydroxy-C₁-C₆alkyloxy,
hydroxy-C₆-C₁₀aryl,
hydroxy-C₆-C₁₀aryloxy,
hydroxy-C₁-C₄alkyloxy,
morpholinyl,
NR⁵R⁶,
C(═NR⁵)—NR⁵R⁶, and
pthalamidyl,
where any alkyl or cycloalkyl may be substituted with 1-3 R⁷ and any aryl or heteroaryl may be substituted with 1-3 R⁸;
R⁵ and R⁶ are independently selected from
hydrogen,
het-C₀-C₄alkyl,
het-C₀-C₄alkylcarbonyl,
het-C₀-C₄alkyloxycarbonyl,
het-C₀-C₄alkylaminocarbonyl,
het-C₀-C₄alkylsulfonyl,
C₁-C₄alkyl,
C₁-C₄alkylcarbonyl,
C₀-C₄alkyl-C₆-C₁₀aryl carbonyl,
C₁-C₄alkylsulfonyl,
C₁-C₄alkyloxy-C₀-C₄alkyl,
C₁-C₄alkyloxy-C₀-C₄alkylcarbonyl,
C₁-C₄alkylamino-C₀-C₄alkylcarbonyl,
C₆-C₁₀aryl-C₀-C₄alkyl,
C₆-C₁₀aryl-C₀-C₄alkylcarbonyl,
C₆-C₁₀aryl-C₀-C₄alkyloxycarbonyl,
C₆-C₁₀aryl-C₀-C₄alkylamino-C₀-C₄alkylcarbonyl,
C₆-C₁₀aryl-C₀-C₄alkylsulfonyl,
C₀-C₄alkylaminocarbonyl,
C₆-C₁₀aryl-aminocarbonyl,
C₃-C₁₀ cycloalkyl,
C₃-C₁₀ cycloalkylcarbonyl,
C₆-C₁₀aryl-C₃-C₁₀ cycloalkylcarbonyl,
C(═NH)—NH₂,
acetyl,
benzoyl,
morpholino-C₁-C₄alkyl, and
C₁-C₄alkylcarbonyl;
R⁵ and R¹ together with the nitrogen to which they are bonded may form a heterocycle containing up to three heteroatoms selected from N, S and O, where the heterocycle may be substituted with R³ and R⁴;
R⁵ and R⁶ together with the nitrogen to which they are bonded may for a morpholinyl or piperizinyl,
R⁷ is selected from
halo(F, Cl, Br and I),
carbonyl,
hydroxy,
nitro,
cyano,
methylsulfonyl,
aminothiocyanate, and
benzoyl;
R⁸ is selected from
amino,
di-(C₁-C₄alkyl)amino-C₁-C₄alkyl,
aminocarbonyl,
aminosulfonyl,
methoxy,
hydroxy,
C₁-C₄alkyl,
C₁-C₄alkylthio,
C₁-C₄alkyloxycarbonyl,
C₁-C₄alkylcarbonylamino,
carboxy,
carboxyC₁-C₄alkyl,
carboxyC₁-C₄alkylthio,
acetamide,
acetyl,
halo,
nitro,
cyano,
trifluoromethyl, and
formyl; and
R⁹ is selected from
Hydrogen and
—C(═O)NHR¹.

70. The library of claim 69 wherein the compounds are represented by formula III 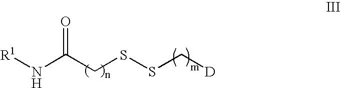
where the substituents are as defined in claim 69.

71. The library of claim 69 wherein the compounds are represented by formula IV 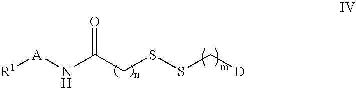
where the substituents are as defined in claim 69.

72. The library of claim 69 wherein the compounds are represented by formula V 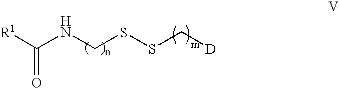
where the substituents are as defined in claim 69.

73. The library of claim 69 wherein the compounds are represented by formula VI 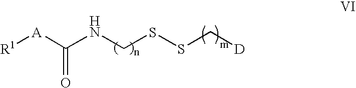
where the substituents are as defined in claim 69.

74-77. (canceled)

78. The library of claim 69 wherein the compounds are mass encoded, the mass of each compound differing from other compounds in the library by at least 5 daltons.

79-90. (canceled)


SEQUENCE LISTING (85 sequences)

SEQ ID NO: 1 - DNA, 35 bp, Artificial Sequence
  Oligonucleotide
ttccatatgtctggaatatccctggacaacagtta

SEQ ID NO: 2 - DNA, 36 bp, Artificial Sequence
  Oligonucleotide
aaggaattcttagtctgtctcaatgccacagtccag

SEQ ID NO: 3 - DNA, 33 bp, Artificial Sequence
  Oligonucleotide
ttccatatgagtggtgttgatgatgacatggcg

SEQ ID NO: 4 - DNA, 39 bp, Artificial Sequence
  Oligonucleotide
aaggaattcttagtgataaaaatagagttcttttgtgag

SEQ ID NO: 5 - DNA, 21 bp, Artificial Sequence
  Primer
ctttgcatgacaagtagcgtc

SEQ ID NO: 6 - DNA, 21 bp, Artificial Sequence
  Primer
gccatccttacaatttcgcca

SEQ ID NO: 7 - DNA, 21 bp, Artificial Sequence
  Primer
agcgtcaaagcaaaaggactc

SEQ ID NO: 8 - DNA, 21 bp, Artificial Sequence
  Primer
ctggatgaaacaggagccatc

SEQ ID NO: 9 - DNA, 21 bp, Artificial Sequence
  Primer
tcgccaagaacaataaccagg

SEQ ID NO: 10 - DNA, 21 bp, Artificial Sequence
  Primer
ttcttcaccacagctcagaag

SEQ ID NO: 11 - DNA, 21 bp, Artificial Sequence
  Primer
gccacagtcacattctgtacc

SEQ ID NO: 12 - DNA, 21 bp, Artificial Sequence
  Primer
ccgagatgtacatccagtgct

SEQ ID NO: 13 - DNA, 21 bp, Artificial Sequence
  Primer
atctgtaccacaccgagatgt

SEQ ID NO: 14 - DNA, 43 bp, Artificial Sequence
  Forward primer
ggaattccatatggcacctacttcaagttctacaaagaaaaca

SEQ ID NO: 15 - DNA, 39 bp, Artificial Sequence
  Reverse primer
ccgctcgagtcaagttagtgttgagatgatgctttgaca

SEQ ID NO: 16 - DNA, 25 bp, Artificial Sequence
  Oligonucleotide
ttaattccattcaaaatcatctgta

SEQ ID NO: 17 - DNA, 48 bp, Artificial Sequence
  Mutagenic oligonucleotides
ggtgagtttgggattcttgtaacaattaattccattcaaaatcatctg

SEQ ID NO: 18 - DNA, 37 bp, Artificial Sequence
  Mutagenic oligonucleotides
ggtgagtttgggattcttacaattattaattccattc

SEQ ID NO: 19 - DNA, 36 bp, Artificial Sequence
  Mutagenic oligonucleotides
cctggtgagtttgggacacttgtaattattaattcc

SEQ ID NO: 20 - DNA, 33 bp, Artificial Sequence
  Mutagenic oligonucleotides
ggtgagtttgggattacagtaattattaattcc

SEQ ID NO: 21 - DNA, 40 bp, Artificial Sequence
  Mutagenic oligonucleotides
gcatcctggtgagacagggattcttgtaattattaattcc

SEQ ID NO: 22 - DNA, 34 bp, Artificial Sequence
  Mutagenic oligonucleotides
cttaaatgtgagcatacaggtgagtttgggattc

SEQ ID NO: 23 - DNA, 32 bp, Artificial Sequence
  Mutagenic oligonucleotides
gggcatgtaaaacttacatgtgagcatcctgg

SEQ ID NO: 24 - DNA, 32 bp, Artificial Sequence
  Mutagenic oligonucleotides
cttgggcatgtaaaaacaaaatgtgagcatcc

SEQ ID NO: 25 - DNA, 34 bp, Artificial Sequence
  Mutagenic oligonucleotides
ggccttcttgggcatacaaaacttaaatgtgagc

SEQ ID NO: 26 - DNA, 32 bp, Artificial Sequence
  Mutagenic oligonucleotides
ctcaaacctctggagtgtgtgctaaatttagc

SEQ ID NO: 27 - DNA, 34 bp, Artificial Sequence
  Mutagenic oligonucleotides
gtttttgctttgagcacaatttagcacttcctcc

SEQ ID NO: 28 - DNA, 43 bp, Artificial Sequence
  Mutagenic oligonucleotides
cctgggtcttaagtgaaaacatttgctttgagctaaatttagc

SEQ ID NO: 29 - DNA, 63 bp, Artificial Sequence
  Mutagenic oligonucleotides
gggcatgtaaaaacaaaatgtgagcatcctggtgagtttgggattcttacaattattaattcc

SEQ ID NO: 30 - DNA, 17 bp, Artificial Sequence
  Forward primer
aatacgactcactatag

SEQ ID NO: 31 - DNA, 20 bp, Artificial Sequence
  Reverse primer
tagttattgctcagcggtgg

SEQ ID NO: 32 - DNA, 30 bp, Artificial Sequence
  Primer
ttactcagacttatttgtttcatagctcta

SEQ ID NO: 33 - DNA, 30 bp, Artificial Sequence
  Primer
gaaattagtgactggatatattaactggat

SEQ ID NO: 34 - DNA, 39 bp, Artificial Sequence
  Forward primer
ccggaattcatgaaagtgttactcagacttatttgtttc

SEQ ID NO: 35 - DNA, 42 bp, Artificial Sequence
  Reverse primer
ccgctcgagtcacttctggaaattagtgactggatatattaa

SEQ ID NO: 36 - DNA, 17 bp, Artificial Sequence
  Forward primer
aatacgactcactatag

SEQ ID NO: 37 - DNA, 20 bp, Artificial Sequence
  Reverse primer
tagttattgctcagcggtgg

SEQ ID NO: 38 - DNA, 20 bp, Artificial Sequence
  Forward primer
tattccggattattcatacc

SEQ ID NO: 39 - DNA, 21 bp, Artificial Sequence
  Reverse primer
cctctacaaatgtggtatggc

SEQ ID NO: 40 - protein, 21 aa, Artificial Sequence
  Primer
ETPFTWEESNAYYWQPYALPL

SEQ ID NO: 41 - DNA, 25 bp, Artificial Sequence
  Mutagenic oligonucleotides
taaaattattttacattcacgttcc

SEQ ID NO: 42 - DNA, 27 bp, Artificial Sequence
  Mutagenic oligonucleotides
tgacactaaaatacatttttcttcacg

SEQ ID NO: 43 - DNA, 26 bp, Artificial Sequence
  Mutagenic oligonucleotides
atttgcagatgaacataaaattattt

SEQ ID NO: 44 - DNA, 24 bp, Artificial Sequence
  Mutagenic oligonucleotides
gggtagtttctgacaaaatatggc

SEQ ID NO: 45 - DNA, 27 bp, Artificial Sequence
  Mutagenic oligonucleotide
aacgggtagtttacacttaaatatggc

SEQ ID NO: 46 - DNA, 24 bp, Artificial Sequence
  Mutagenic oligonucleotide
catataagggcaacaaagtcctcc

SEQ ID NO: 47 - DNA, 24 bp, Artificial Sequence
  Mutagenic oligonucleotide
tttaaaaaaacacatataagggca

SEQ ID NO: 48 - DNA, 30 bp, Artificial Sequence
  Forward primer
gcccatatggagatggaaaaggagttcgag

SEQ ID NO: 49 - DNA, 40 bp, Artificial Sequence
  Reverse primer
gcgacgcgaattcttaattgtgtggctccaggattcgttt

SEQ ID NO: 50 - DNA, 34 bp, Artificial Sequence
  Reverse primer
tgccggaattccttagtcctcgtgggaaagctcc

SEQ ID NO: 51 - DNA, 27 bp, Artificial Sequence
  Mutagenesis oligonucleotides
tgggaagtcactgcattcatgtcggat

SEQ ID NO: 52 - DNA, 27 bp, Artificial Sequence
  Mutagenesis oligonucleotides
gatgcctgcactggagtgcaccacaac

SEQ ID NO: 53 - DNA, 27 bp, Artificial Sequence
  Mutagenesis oligonucleotides
cttggccactctagatgggaagtcact

SEQ ID NO: 54 - DNA, 27 bp, Artificial Sequence
  Mutagenesis oligonucleotides
ccaaaagtgaccgactgtgttaggcaa

SEQ ID NO: 55 - DNA, 27 bp, Artificial Sequence
  Mutagenesis oligonucleotides
agggactccaaagcaaggccatgtggt

SEQ ID NO: 56 - DNA, 27 bp, Artificial Sequence
  Mutagenesis oligonucleotides
tctacatgggaagcaactggcttcatg

SEQ ID NO: 57 - DNA, 27 bp, Artificial Sequence
  Mutagenesis oligonucleotides
tctagatgggaagcaactggcttcatg

SEQ ID NO: 58 - DNA, 27 bp, Artificial Sequence
  Mutagenesis oligonucleotides
aaagggactgacgcatctgtacctatt

SEQ ID NO: 59 - DNA, 27 bp, Artificial Sequence
  Mutagenesis oligonucleotides
ttcagggactccacagtcaggccatgt

SEQ ID NO: 60 - DNA, 27 bp, Artificial Sequence
  Mutagenesis oligonucleotides
ccgactatggtcacagggactgacgtc

SEQ ID NO: 61 - DNA, 27 bp, Artificial Sequence
  Mutagenesis oligonucleotides
gtcactggcttcacatcggatatcctg

SEQ ID NO: 62 - DNA, 27 bp, Artificial Sequence
  Mutagenesis oligonucleotides
gtattgtgcgcaacataacgaaccttt

SEQ ID NO: 63 - DNA, 27 bp, Artificial Sequence
  Mutagenesis oligonucleotides
gttcttaggaagacaggccactctaca

SEQ ID NO: 64 - DNA, 27 bp, Artificial Sequence
  Mutagenesis oligonucleotides
gttcttaggaagacaggccactctaga

SEQ ID NO: 65 - DNA, 27 bp, Artificial Sequence
  Mutagenesis oligonucleotides
ctggatcagcccacaccgaaacttcct

SEQ ID NO: 66 - DNA, 27 bp, Artificial Sequence
  Mutagenesis oligonucleotides
ctggtcggctgtacagatcagccccat

SEQ ID NO: 67 - DNA, 27 bp, Artificial Sequence
  Mutagenesis oligonucleotides
gggactgacgtcacagtacctatttcg

SEQ ID NO: 68 - DNA, 27 bp, Artificial Sequence
  Mutagenesis oligonucleotides
atggtcaaagggacagacgtctctgta

SEQ ID NO: 69 - DNA, 27 bp, Artificial Sequence
  Mutagenesis oligonucleotides
gtcaaagggactgcagtctctgtacct

SEQ ID NO: 70 - DNA, 27 bp, Artificial Sequence
  Mutagenesis oligonucleotides
actgacgtctctgcacctatttcggtt

SEQ ID NO: 71 - DNA, 17 bp, Artificial Sequence
  Primer
aatacgactcactatag

SEQ ID NO: 72 - DNA, 20 bp, Artificial Sequence
  Reverse primer
tagttattgctcagcggtgg

SEQ ID NO: 73 - DNA, 32 bp, Artificial Sequence
  Reverse primer
gggtttcatatgcacaagtgcgatatcacctt

SEQ ID NO: 74 - DNA, 30 bp, Artificial Sequence
  Reverse primer
ccgctcgagtcagctcgaacactttgaata

SEQ ID NO: 75 - DNA, 27 bp, Artificial Sequence
  Oligonucleotide
agttttgatgatacactgtaaggtgat

SEQ ID NO: 76 - DNA, 26 bp, Artificial Sequence
  Oligonucleotide
ctggttggcttcacacacaggacagg

SEQ ID NO: 77 - DNA, 26 bp, Artificial Sequence
  Oligonucleotide
ctctcatgatcgtgcatagcctttcc

SEQ ID NO: 78 - DNA, 27 bp, Artificial Sequence
  Oligonucleotide
gctgttcaaagtgcagatgatctcctg

SEQ ID NO: 79 - DNA, 26 bp, Artificial Sequence
  Oligonucleotide
cagttgtgttacaggaggcagcaaag

SEQ ID NO: 80 - DNA, 25 bp, Artificial Sequence
  Oligonucleotide
gcagaaggtttcacactcagttgtg

SEQ ID NO: 81 - DNA, 25 bp, Artificial Sequence
  Oligonucleotide
ccttctcagttgtgcacttggaggc

SEQ ID NO: 82 - DNA, 25 bp, Artificial Sequence
  Oligonucleotide
cacaggacaggaacacaagcccgcc

SEQ ID NO: 83 - DNA, 27 bp, Artificial Sequence
  Oligonucleotide
ggctgtagaaacaccggagcacagtcg

SEQ ID NO: 84 - DNA, 24 bp, Artificial Sequence
  Oligonucleotide
gaatatttctcacacatgatcgtc

SEQ ID NO: 85 - DNA, 24 bp, Artificial Sequence
  Oligonucleotide
ctgctctgtgaggcagttcaaagt